(19) United States
(12) Patent Application Publication
(10) Pub. No.: US 2019/0359609 A1
(43) Pub. Date: Nov. 28, 2019

(54) BICYCLIC OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Carlos Manuel Martinez Viturro, Olias del Rey (ES); Gary John Tresadern, Toledo (ES); Manuel Jesús Alcázar-Vaca, Toledo (ES)

(21) Appl. No.: 16/469,685

(22) PCT Filed: Dec. 15, 2017
(86) PCT No.: PCT/EP2017/083125

§ 371 Date: Jun. 14, 2019

(30) Foreign Application Priority Data
Dec. 16, 2016 (EP) 16204834.2

(51) Int. Cl. C07D 471/04; C07D 491/056

(52) U.S. Cl. CPC C07D 471/04; C07D 491/056

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

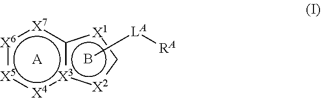

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield 0-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA)O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in then-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (Aβ) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2015/164508 A1 (DART Neuroscience LLC) describes [1,2,4]-triazolo-[1,5-a]pyrimidin-7-yl derivatives as PDE2 inhibitors. WO2016/030443 (Asceneuron SA) describes in particular 1,4-disubstituted piperidine and piperazine derivatives as OGA inhibitors.

There is still a need for OGA inhibitors with an advantageous balance of properties, for example with improved potency, better selectivity, brain penetration and/or better side effect profile.

### SUMMARY OF THE INVENTION

It has now been found that compounds bearing a 1,3-disubstituted piperidine or piperazine or a 2,4-disubstituted morpholine bound to the 5-membered ring of a 9-membered bicyclic heteroaryl exhibit OGA inhibitory activity and a good balance of properties. Thus, in one aspect, the present invention is directed to compounds of Formula (I)

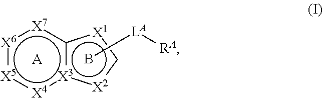

and the tautomers and the stereoisomeric forms thereof, wherein
A-B represent a 9-membered bicyclic heteroaryl system having from 1 to 4 nitrogen atoms, wherein
X¹ and X² are each independently selected from the group consisting of C, CR^{x}, N, and NR^{y}; and

### X³ is C or N;

X⁴, X⁵, X⁶, and X⁷ are each independently selected from the group consisting of CR^{x} and N;
with the proviso that at least one of X² and X³ is N or in the case of X², is N or NR^{y}; wherein each R^{x}, when present, is independently selected from the group consisting of hydrogen; halo; —CN; C₁₋₄alkyl optionally substituted with 1, 2 or 3 independently selected halo substituents; and C₁₋₄alkyloxy optionally substituted with 1, 2 or 3 independently selected halo substituents;
each R^{y}, when present, is independently selected from the group consisting of hydrogen and C₁₋₄alkyl optionally substituted with 1, 2 or 3 independently selected halo substituents;
L^{A} is bound to any available carbon or nitrogen atom at the 5-membered B ring of the A-B bicycle, and is selected from a bond and CHR¹; wherein
R¹ is selected from the group consisting of hydrogen and C₁₋₄alkyl optionally substituted with 1, 2 or 3 independently selected halo substituents;
R^{A} is a radical selected from the group consisting of (a-1), (a-2) and (a-3)

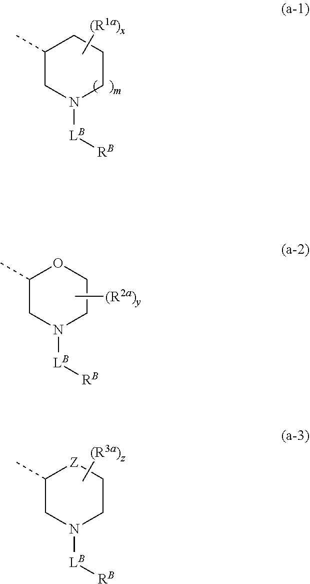

wherein
m represents 0 or 1;
x, y and z, each independently represent 0, 1 or 2;
each R^{1a} and R^{2a} when present, is bound to any available carbon atom and is independently selected from the group consisting of halo and C₁₋₄alkyl optionally substituted with 1, 2, or 3 independently selected halo substituents; or two R^{1a}, or two R^{2a} substituents are bound to the same carbon atom and together form a cyclopropylidene radical;
Z is N when substituted with R^{3a}, or NH;
each R^{3a} is bound to any available carbon or nitrogen atom when present and is independently selected from C₁₋₃alkyl optionally substituted with 1, 2 or 3 independently selected halo substituents; or two R^{3a} are bound to the same carbon atom and together form a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} is a radical selected from the group consisting of (b-1), (b-2), (b-3), (b-4), (b-5), (b-6), (b-7), (b-8), (b-9), (b-10), and (b-11):

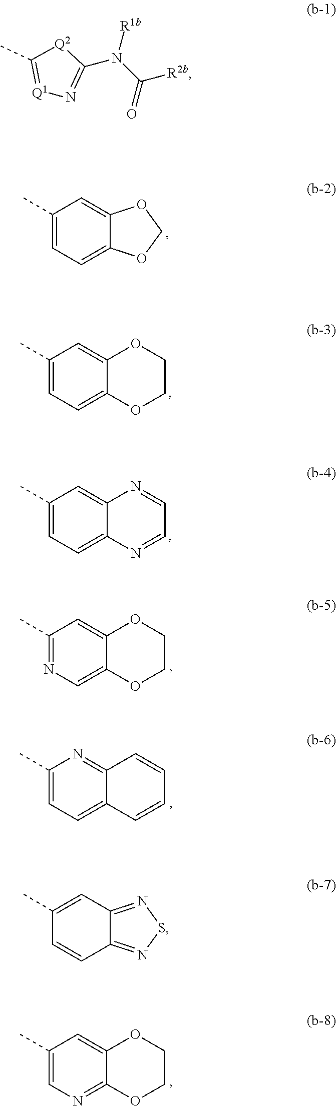

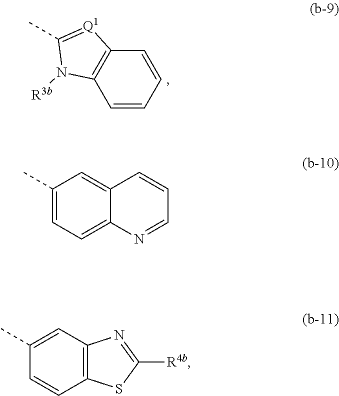

wherein

### Q¹ is CH or N;

Q² is O, NR⁴a or S;
R^{4a}, R^{1b}, R^{3b} and R^{4b} are each independently selected from H and C₁₋₄alkyl; and
R^{2b} is C₁₋₄alkyl;
or -L^{B}-R^{B} is (b-12)

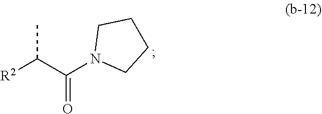

and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a prophylactically or a therapeutically effective amount of any of the compounds or pharmaceutical compositions described above.

Further exemplifying the invention are methods of inhibiting OGA, comprising administering to a subject in need thereof a prophylactically or therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I) as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or maybe useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as referred to herein, and the tautomers and the stereoisomeric forms thereof, wherein

X¹ is selected from the group consisting of CR^{x}, N, and NR^{y};
X² is N or NR^{y};

### X³ is C or N;

X⁴, X⁵, X⁶, and X⁷ are each independently selected from the group consisting of CR^{x} and N;
wherein each R^{x}, when present, is independently selected from the group consisting of hydrogen; halo; —CN; C₁₋₄alkyl optionally substituted with 1, 2 or 3 independently selected halo substituents; and C₁₋₄alkyloxy optionally substituted with 1, 2 or 3 independently selected halo substituents;
each R^{y}, when present, is independently selected from the group consisting of hydrogen and C₁₋₄alkyl optionally substituted with 1, 2 or 3 independently selected halo substituents;
L^{A} is bound to any available carbon or nitrogen atom at the 5-membered B ring of the A-B bicycle, and is selected from a bond and CHR¹; wherein
R¹ is selected from the group consisting of hydrogen and C₁₋₄alkyl optionally substituted with 1, 2 or 3 independently selected halo substituents;
R^{A} is a radical selected from the group consisting of (a-1), (a-2) and (a-3), wherein m represents 0 or 1;
x, y and z, each independently represent 0 or 1;
each R^{1a} and R^{2a} when present, is bound to any available carbon atom and is independently selected from the group consisting of halo and C₁₋₄alkyl optionally substituted with 1, 2, or 3 independently selected halo substituents; or two R^{1a}, or two R^{2a} substituents are bound to the same carbon atom and together form a cyclopropylidene radical;
Z is N when substituted with R^{3a}, or NH;
each R^{3a} is bound to any available carbon or nitrogen atom when present and is independently selected from C₁₋₃alkyl optionally substituted with 1, 2 or 3 independently selected halo substituents; or two R^{3a} are bound to the same carbon atom and together form a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and R^{B} is a radical selected from the group consisting of (b-1), (b-2), (b-3), (b-4), (b-5), (b-6), (b-7), (b-8), (b-9), (b-10), and (b-11):

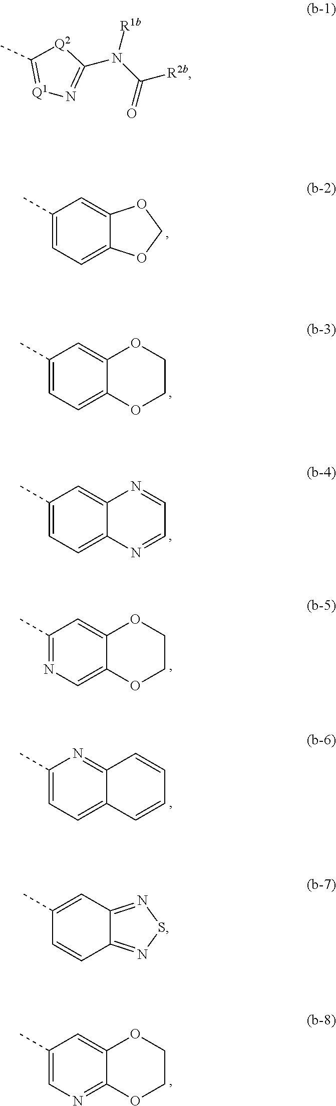

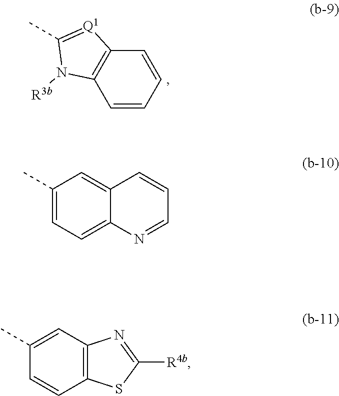

wherein

### Q¹ is CH or N;

Q² is O, NR⁴a or S;
R^{4a}, R^{1b}, R^{3b} and R^{4b} are each independently selected from H and C₁₋₄alkyl; and
R^{2b} is C₁₋₄alkyl;
or -L^{B}-R^{B} is (b-12)

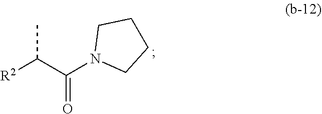

and the pharmaceutically acceptable salts and the solvates thereof.

In a further embodiment, the invention is directed to compounds of Formula (I) as referred to herein, and the tautomers and the stereoisomeric forms thereof, wherein

X¹ is selected from the group consisting of CR^{x}, N, and NW;

### X² is N or NW;

### X³ is C;

X⁴, X⁵, X⁶, and X⁷ are each independently selected from the group consisting of CR^{x} and N;
wherein each R^{x}, when present, is independently selected from the group consisting of hydrogen; halo; C₁₋₄alkyl optionally substituted with 1, 2 or 3 independently selected halo substituents; and C₁₋₄alkyloxy optionally substituted with 1, 2 or 3 independently selected halo substituents;
each R^{y}, when present, is independently selected from the group consisting of hydrogen and C₁₋₄alkyl;
L^{A} is bound to any available carbon or nitrogen atom at the 5-membered B ring of the A-B bicycle, and is selected from a bond and CH₂;
R^{A} is a radical selected from the group consisting of (a-1), (a-2) and (a-3), wherein m represents 0 or 1;
x, y and z, each independently represent 0 or 1;
each R^{1a} and R^{2a} when present, is bound to any available carbon atom and is independently selected from the group consisting of halo and C₁₋₄alkyl optionally substituted with 1, 2, or 3 independently selected halo substituents;

### Z is NH;

each R^{3a} is bound to any available carbon atom when present and is independently selected from C₁₋₃alkyl optionally substituted with 1, 2 or 3 independently selected halo substituents;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} is a radical selected from the group consisting of (b-1), (b-2), (b-3), (b-4), (b-5), (b-6), (b-7), (b-8), (b-9), (b-10), and (b-11):

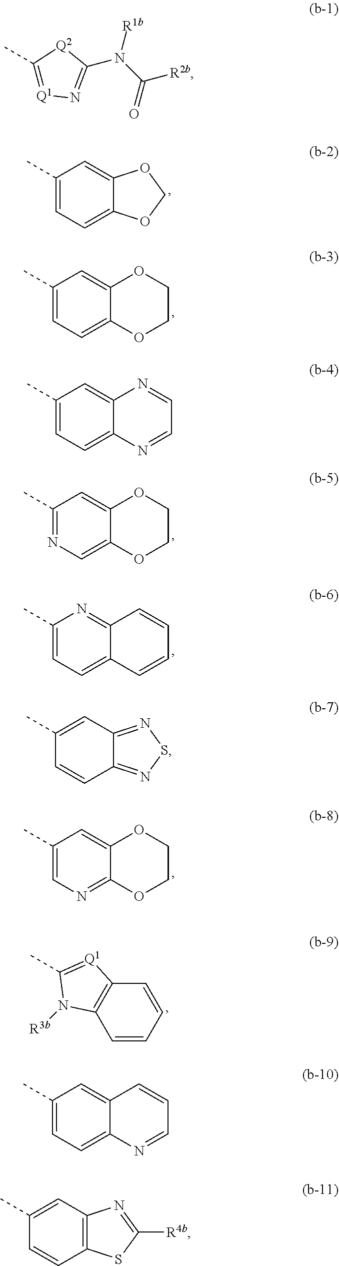

wherein

### Q¹ is CH or N;

### Q² is S;

R^{4a}, R^{1b}, R^{3b} and R^{4b} are each independently selected from H and C₁₋₄alkyl; and
R^{2b} is C₁₋₄alkyl;
or -L^{B}-R^{B} is (b-12′)

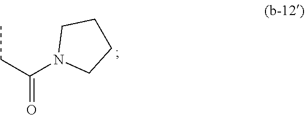

and the pharmaceutically acceptable salts and the solvates thereof.

In a further embodiment, the invention is directed to compounds of Formula (I) as referred to herein, and the tautomers and the stereoisomeric forms thereof, wherein

X¹ is selected from the group consisting of CR^{x}, N, and NR^{y};
X² is N or NR^{y};

### X³ is C;

X⁴, X⁵, X⁶, and X⁷ are each independently selected from the group consisting of CR^{x} and N;
wherein each R^{x}, when present, is independently selected from the group consisting of hydrogen; halo; C₁₋₄alkyl optionally substituted with 1, 2 or 3 independently selected halo substituents; and C₁₋₄alkyloxy optionally substituted with 1, 2 or 3 independently selected halo substituents;
each R^{y}, when present, is independently selected from the group consisting of hydrogen and C₁₋₄alkyl;
L^{A} is bound to any available carbon or nitrogen atom at the 5-membered B ring of the A-B bicycle, and is selected from a bond and CH₂;
R^{A} is a radical selected from the group consisting of (a-1), (a-2) and (a-3), wherein m represents 0 or 1;
x, y and z, each independently represent 0 or 1;
each R^{1a} and R^{2a} when present, is C₁₋₄alkyl bound to any available carbon atom;

### Z is NH;

each R^{3a} when present, is C₁₋₃alkyl bound to any available carbon;
L^{B} is selected from the group consisting of >CHR² and >SO₂;
wherein R² is selected from the group consisting of hydrogen and C₁₋₄alkyl; and
R^{B} is a radical selected from the group consisting of (b-1), (b-2), (b-3), (b-4), (b-5), (b-6), (b-7), (b-8), (b-9), (b-10), and (b-11):

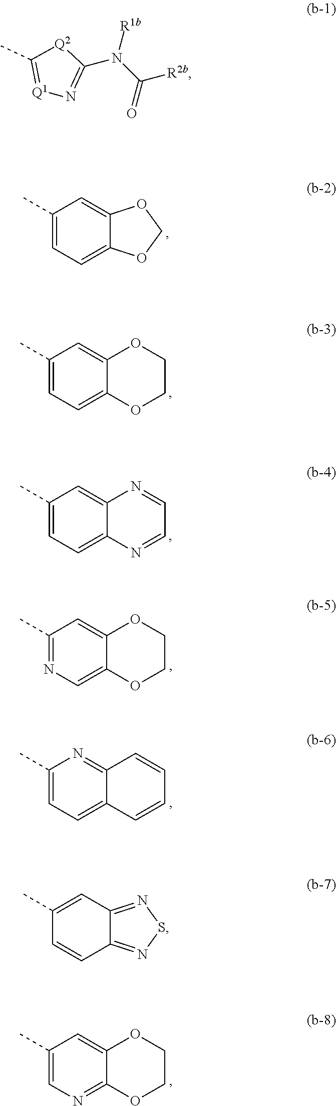

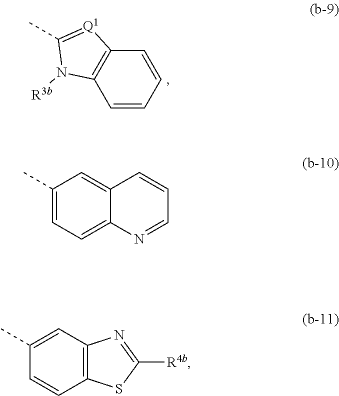

wherein

### Q¹ is CH or N;

### Q² is S;

R^{4a}, R^{1b}, R^{3b} and R^{4b} are each independently selected from H and CH₃; and
R^{2b} is C₁₋₄alkyl;
or -L^{B}-R^{B} is (b-12′)

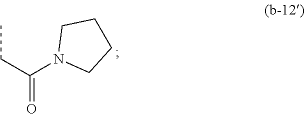

and the pharmaceutically acceptable salts and the solvates thereof.

In an embodiment, the compounds of Formula (I) are in particular compounds of Formula (I-A),

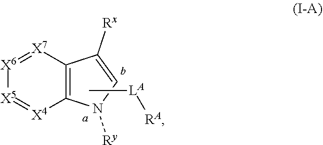

wherein
one of X⁴, X⁵, X⁶ or X⁷ is N and the remaining are CH;
R^{x} is selected from the group consisting of hydrogen; halo; and C₁₋₄alkyl optionally substituted with 1, 2 or 3 independently selected halo substituents;
R^{y} is absent when L^{A} is bound at position a of the 5-membered ring of the A-B 9-membered bicyclic heteroaryl system or is selected from hydrogen and C₁₋₄alkyl when L^{A} is bound at position b of the 5-membered ring of the A-B 9-membered bicyclic heteroaryl system;
and all other variables are as described in Formula (I) herein.

In a further embodiment, the compounds of Formula (I-A) are in particular compounds of Formula (I-A′),

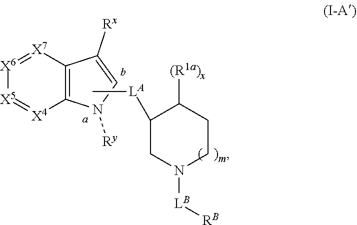

wherein
one of X⁴, X⁵, X⁶ or X⁷ is N and the remaining are CH;
R^{x} is selected from the group consisting of hydrogen; halo; and C₁₋₄alkyl optionally substituted with 1, 2 or 3 independently selected halo substituents;
R^{y} is absent when L^{A} is bound at position a of the 5-membered ring or is selected from hydrogen and C₁₋₄alkyl when L^{A} is bound at position b of the 5-membered ring;
L^{A} is a bond or CH₂;
m is 0 or 1;
x is 0 or 1;
R^{1a} when present is C₁₋₄alkyl;
L^{B} is selected from the group consisting of >CH₂, >CH(CH₃), and >SO₂; in particular >CH₂ and >CH(CH₃); and
R^{B} is (b-1) or (b-4) as described in Formula (I) herein.

In a further embodiment, the compounds of Formula (I) are in particular compounds of Formula (I-B),

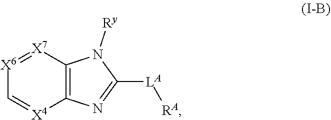

wherein
one of X⁴ or X⁷ is N and the other X⁷ or X⁴ is CH;
X⁶ is N or CR^{x} wherein R^{x} is selected from the group consisting of hydrogen; halo;
C₁₋₄alkyl optionally substituted with 1, 2 or 3 independently selected halo substituents; and C₁₋₄alkyloxy;
R^{y} is selected from hydrogen and C₁₋₄alkyl;
and all other variables are as described in Formula (I) herein.

In a further embodiment, the compounds of Formula (I-B) are in particular compounds of Formula (I-B′),

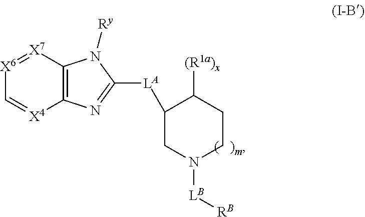

wherein
one of X⁴ or X⁷ is N and the other X⁷ or X⁴ is CH;
X⁶ is N or CR^{x} wherein R^{x} is selected from the group consisting of hydrogen; halo;
C₁₋₄alkyl optionally substituted with 1, 2 or 3 independently selected halo substituents; and C₁₋₄alkyloxy;
R^{y} is selected from hydrogen and C₁₋₄alkyl;
L^{A} is a bond or CH₂;
m is 0 or 1;
x is 0 or 1;
R^{1a} when present is C₁₋₄alkyl;
L^{B} is selected from the group consisting of >CH₂, >CH(CH₃), and >SO₂; in particular >CH₂ and >CH(CH₃); and
R^{B} is (b-1) or (b-4) as described in Formula (I) herein.

In a further embodiment, the A-B 9-membered bicyclic heteroaryl system is selected from (ab-1), (ab-2), (ab-3), (ab-4), (ab-5), (ab-6), (ab-7), and (ab-8):

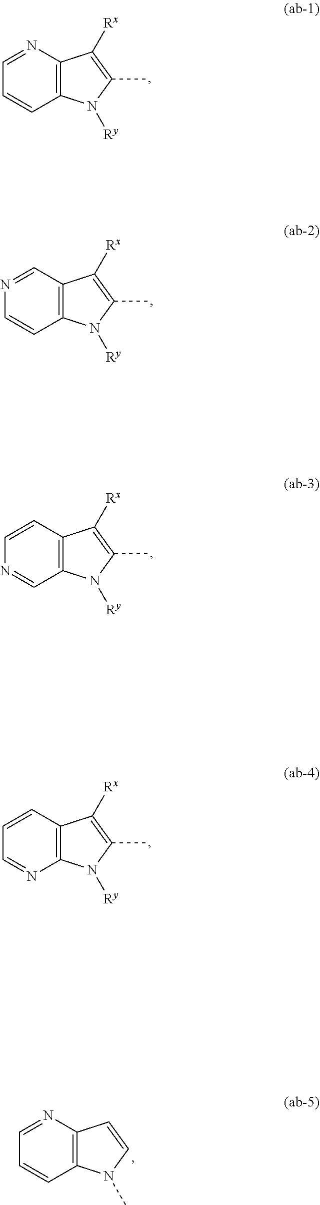

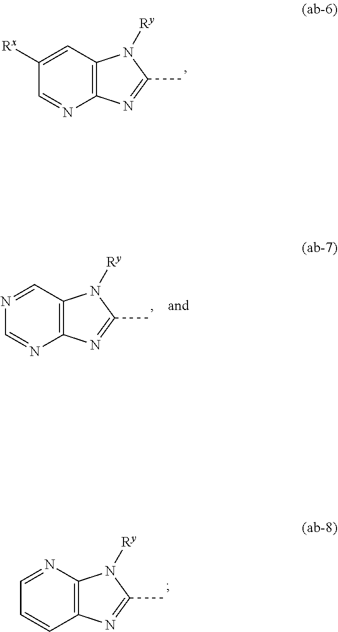

and all other variables are as described in Formula (I), (I-A), (I-A′), (I-B), or (I-B′) herein.

In a particular embodiment, R^{x} is selected from the group consisting of H, CH₃ and CF₃ and all other variables are as defined in any one of Formulae (I), (I-A), (I-B), (I-A′) and (I-B′).

In another embodiment, R^{y} is H or CH₃ and all other variables are as defined in any one of Formulae (I), (I-A), (I-B), (I-A′) and (I-B′).

In another embodiment, x is 0 or 1, y is 0 and z is 0, and all other variables are as defined in any one of Formulae (I), (I-A), (I-B), (I-A′) and (I-B′).

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds according to Formula (I-a), can be prepared by reacting an intermediate compound of Formula (II-a) with a compound of Formula (XI) according to reaction scheme (1). The reaction is performed in a suitable reaction-inert solvent, such as, for example, dichloromethane, in the presence of a suitable base, such as, for example, triethylamine, under thermal conditions 0° C. or room temperature, for example for 1 hour. In reaction scheme (1) all variables are defined as in Formula (I) and

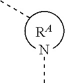

represents the optionally substituted heterocyclyl moiety at R^{A} (i.e., pyrrolidinyl or piperidinyl from (a-1), morpholinyl from (a-2) or piperazinyl from (a-3), as defined herein)

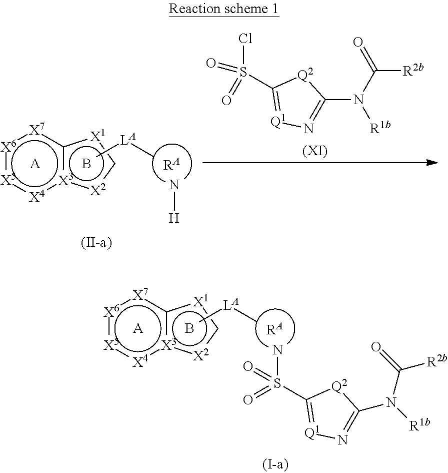

### Experimental Procedure 2

Additionally, final compounds of Formula (I-b) can be prepared by reacting an intermediate compound of Formula (II-a) with a compound of Formula (XII) according to reaction scheme (2). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (2) all variables are defined as in Formula (I) and

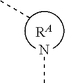

represents the optionally substituted heterocyclyl moiety at R^{A} (i.e., pyrrolidinyl or piperidinyl from (a-1), morpholinyl from (a-2) or piperazinyl from (a-3), as defined herein)

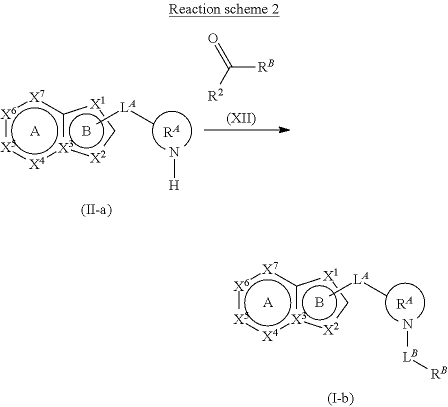

### Experimental Procedure 3

Additionally, final compounds of Formula (I-b) can be prepared by reacting an intermediate compound of Formula (II-a) with a compound of Formula (XIII) according to reaction scheme (3). The reaction is performed in a suitable reaction-inert solvent, such as, for example, acetonitrile, a suitable base, such as, for example, triethylamine or diisopropylethylamine, under thermal conditions, such as, 0° C. or room temperature, or 75° C., for example for 1 hour or 24 hours. In reaction scheme (3) all variables are defined as in Formula (I), halo is chloro, bromo or iodo and

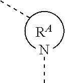

represents the optionally substituted heterocyclyl moiety at R^{A} (i.e., pyrrolidinyl or piperidinyl from (a-1), morpholinyl from (a-2) or piperazinyl from (a-3), as defined herein)

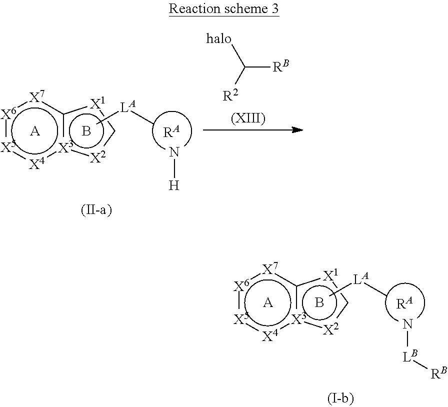

### Experimental Procedure 4

Additionally, final compounds of Formula (I-b) can be prepared by intramolecular cyclization of an intermediate compound of Formula (II-b) according to reaction scheme (4). The reaction is performed in a suitable reaction-inert solvent, such as, for example, N-methylpyrrolidone, a suitable base, such as, for example, potassium tert-butoxide, under thermal conditions, such as, for example, room temperature, for example for 24 hours. In reaction scheme (4) all variables are defined as in Formula (I), and X¹ is CH, X³ is C, L^{A} is a bond and

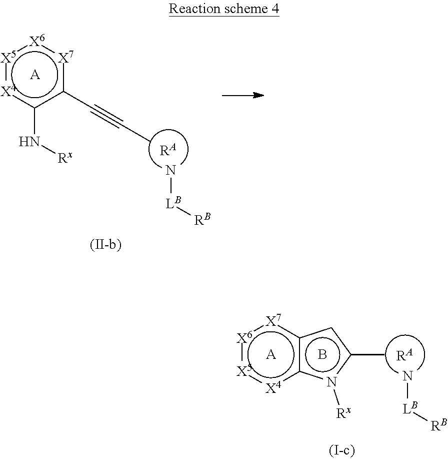

represents the optionally substituted heterocyclyl moiety at R^{A} (i.e., pyrrolidinyl or piperidinyl from (a-1), morpholinyl from (a-2) or piperazinyl from (a-3), as defined herein)

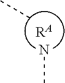

### Experimental Procedure 5

Additionally, final compounds of Formula (I-d) can be prepared by alkylation of a final compound of Formula (II-c) with a compound of Formula (XIV) according to reaction scheme (5). The reaction is performed with an alkylating agent, such as, methyliodide, in a suitable reaction-inert solvent, such as, tetrahydrofuran, a suitable base, such as, for example, sodium hydride, under thermal conditions, such as, for example, 0° C. or room temperature, for example for 24 hours. In reaction scheme (5) all variables are defined as in Formula (I), and X¹ is NH,

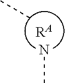

represents the optionally substituted heterocyclyl moiety at R^{A} (i.e., pyrrolidinyl or piperidinyl from (a-1), morpholinyl from (a-2) or piperazinyl from (a-3), as defined herein) and LG is a suitable leaving group such as halo.

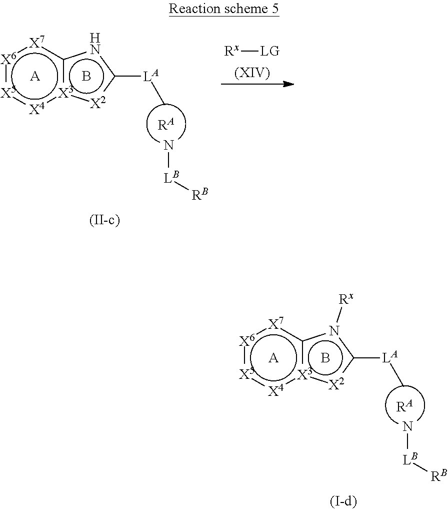

### Experimental Procedure 6

Additionally, final compounds of Formula (I-e) can be prepared by reacting an intermediate compound of Formula (II-a) with a compound of Formula (XVII) followed by reaction of the formed imine derivative with and intermediate compound of Formula (XVIII) according to reaction scheme (6). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. or room temperature, for example for 1 hour or 24 hours. In reaction scheme (6) all variables are defined as in Formula (I), R² is C₁₋₄alkyl, halo is chloro, bromo or iodo and

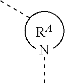

represents the optionally substituted heterocyclyl moiety at R^{A} (i.e., pyrrolidinyl or piperidinyl from (a-1), morpholinyl from (a-2) or piperazinyl from (a-3), as defined herein).

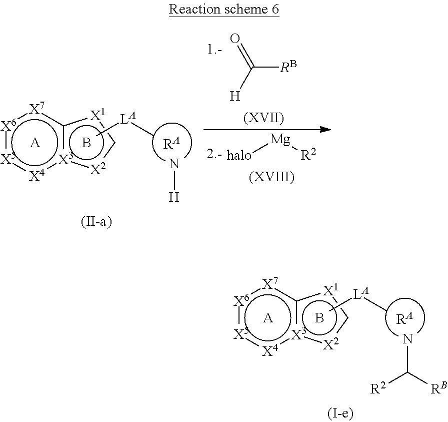

### Experimental Procedure 7

Intermediate compounds of Formula (II-a) can be prepared cleaving a protecting group in an intermediate compound of Formula (III) according to reaction scheme (7). In reaction scheme (7) all variables are defined as in Formula (I),

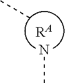

represents the 6-membered optionally substituted heterocyclyl moiety at R^{A} (i.e., pyrrolidinyl or piperidinyl from (a-1), morpholinyl from (a-2) or piperazinyl from (a-3), as defined herein) and PG is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known by the person skilled in the art and comprise but are not limited to: Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

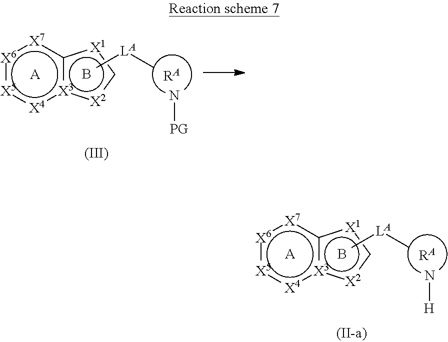

### Experimental Procedure 8

Intermediate compounds of Formula (III-a) can be prepared by intramolecular cyclization of an intermediate compound of Formula (IV) according to reaction scheme (8). The reaction is performed in a suitable reaction-inert solvent, such as, for example, N-methylpyrrolidone or N,N-dimethylformamide, a suitable base, such as, for example, potassium tert-butoxide or sodium hydroxide, under thermal conditions, such as, for example, room temperature, for example for 24 hours. In reaction scheme (8) all variables are defined as in Formula (I), and wherein X¹ is CH, X³ is C, L^{A} is a bond and

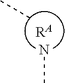

represents the optionally substituted heterocyclyl moiety at R^{A} (i.e., pyrrolidinyl or piperidinyl from (a-1), morpholinyl from (a-2) or piperazinyl from (a-3), as defined herein) and PG is defined as in Formula (III).

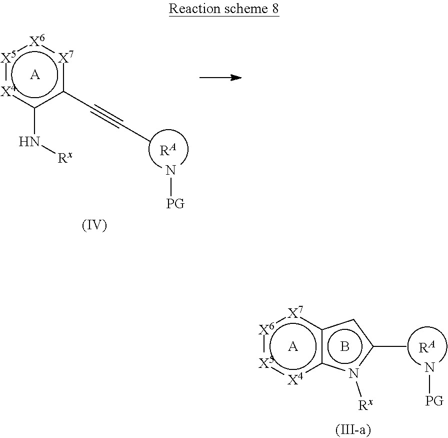

### Experimental Procedure 9

Intermediate compounds of Formula (IV) can be prepared by Sonogashira coupling of an alkyne of Formula (V) with an ortho-halo-aminoheterocycle of Formula (VI) according to reaction scheme (9). The reaction is performed in a suitable reaction-inert solvent, such as, for example, acetonitrile or N,N-dimethylformamide, a suitable base, such as, for example, potassium carbonate or triethylamine, a suitable catalyst, such as for example, Pd(PPh₃)₄ or PdCl₂(PPh₃)₂, and a suitable copper salt, such as for example, copper (I) iodide, under thermal conditions, such as, for example, 100° C., for example for 1 hour. In reaction scheme (9) all variables are defined as in Formula (I),

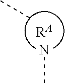

represents the optionally substituted heterocyclyl moiety at R^{A} (i.e., pyrrolidinyl or piperidinyl from (a-1), morpholinyl from (a-2) or piperazinyl from (a-3), as defined herein), halo is chloro, bromo or iodo and PG is defined as in Formula (III).

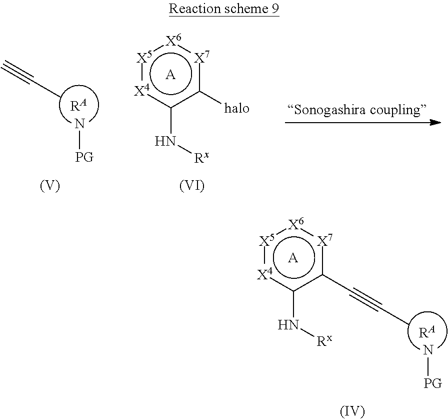

### Experimental Procedure 10

Intermediate compounds of Formula (V) can be prepared reaction of an aldehyde of Formula (VII) with dimethyl-(1-diazo-2-oxypropyl)phosphonate according to reaction scheme (10). The reaction is performed in a suitable reaction-inert solvent, such as, for example, methanol, and a suitable base, such as, for example, potassium carbonate, under thermal conditions, such as, for example, room temperature, for example for 16 hours. In reaction scheme (10) all variables are defined as in Formula (I), and

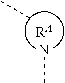

represents the optionally substituted heterocyclyl moiety at R^{A} (i.e., pyrrolidinyl or piperidinyl from (a-1), morpholinyl from (a-2) or piperazinyl from (a-3), as defined herein) and PG is defined as in Formula (III).

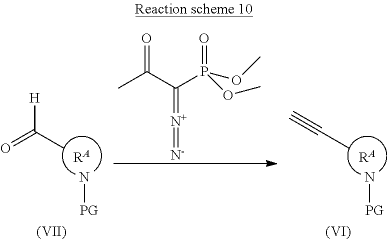

### Experimental Procedure 11

Intermediate compounds of Formula (III-a) can be prepared by “Negishi coupling” reaction of a halo compound of Formula (VIII) with an organozinc compound of Formula (IX) according to reaction scheme (11). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (11) all variables are defined as in Formula (I), halo is preferably iodo and PG is defined as in Formula (III).

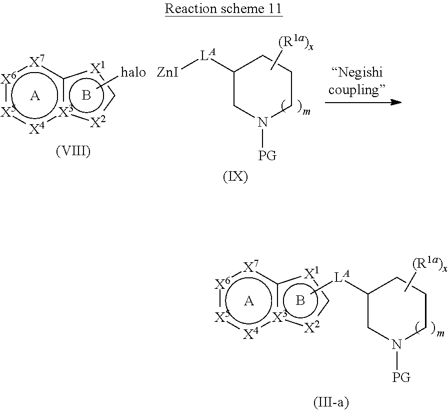

### Experimental Procedure 12

Intermediate compounds of Formula (IX) can be prepared by reaction of a halo compound of Formula (X) with zinc according to reaction scheme (12). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (12) all variables are defined as in Formula (I), halo is preferably iodo, and PG is defined as in Formula (III).

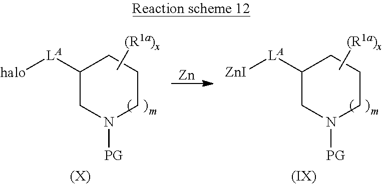

### Experimental Procedure 13

Intermediate compounds of Formula (III-b) can be prepared by alkylation reaction of a compound of Formula (XV) with an intermediate compound compound of Formula (XVI) according to reaction scheme (13). The reaction is performed in a suitable reaction-inert solvent, such as, for example, N,N-dimethylformamide, and a suitable base, such as for example, sodium hydride, under thermal conditions, such as, for example, room temperature, for example for 24 hours. In reaction scheme (13) all variables are defined as in Formula (I), X¹ is NH, L^{A} is CH₂, LG is halo or methylsulfonate,

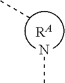

represents the optionally substituted heterocyclyl moiety at R^{A} (i.e., pyrrolidinyl or piperidinyl from (a-1), morpholinyl from (a-2) or piperazinyl from (a-3), as defined herein), and PG is defined as in Formula (III).

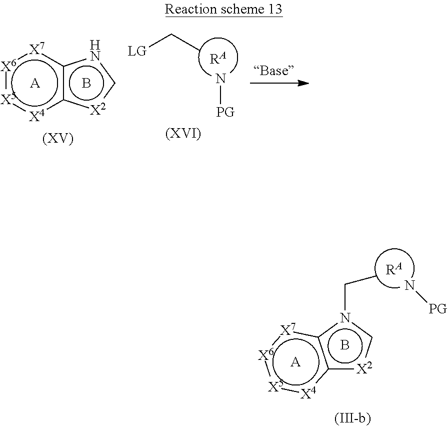

Intermediates of Formulae (VI) (VII), (VIII), (X) (XI), (XII), (XIII), (XIV), (XV), (XVI), (XVII) and (XVIII) are commercially available or can be prepared by know procedures to those skilled in the art.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical states in Alzheimer's and tauopathy diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7: 280-292). Hypothetical models postulate that Aβ accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I′) or (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5′) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “m.p.” means melting point, “min” means minutes, “ACN” means acetonitrile, “aq.” means aqueous, “DMF” means dimethylformamide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “ⁱPrOH” means isopropyl alcohol, “RP” means reversed phase, “Rt” means retention time (in minutes), “[M+H]⁺” means the protonated mass of the free base of the compound, “wt” means weight, “THF” means tetrahydrofuran, “EtOAc” means ethyl acetate, “DCM” means dichloromethane, “MeOH” means methanol, “sat” means saturated, “soltn” means solution, “sol.” means solution, “EtOH” means ethanol, “THF” means tetrahydrofuran, and “NMP” means N-methylpyrrolidone.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “*R” or “*S” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s). Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques.

Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash® 430evo systems from Interchim, or 971-FP systems from Agilent, or Isolera 1SV systems from Biotage.

### A. Preparation of the Intermediates

### Preparation of Intermediates 1, 1a and 1b

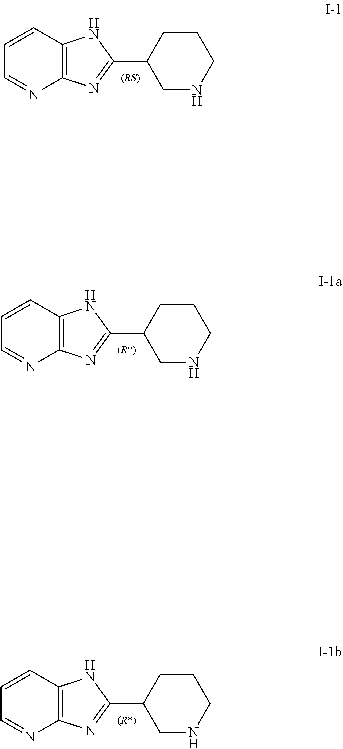 [SMILES: c1cnc2nc(C3CCCNC3)[nH]c2c1 ; c1cnc2nc(C3CCCNC3)[nH]c2c1 ; c1cnc2nc(C3CCCNC3)[nH]c2c1]

A mixture of 2,3-pyridinediamine (1.5 g, 13.75 mmol), nipecotic acid (CAS: 498-95-3, 1.8 g, 13.94 mmol) and polyphosphoric acid (10 mL) was stirred at 180° C. for 16 h. Then the mixture was cooled to rt. Water (100 mL) was added and the mixture was stirred at 50° C. until it became homogeneous. This mixture was then cooled to room temperature and aqueous NaOH (3N) was added until pH=8 was reached. The volatiles were evaporated in vacuo, MeOH was added and the resulting mixture was filtered through a Celite® pad. The filtrate was collected and volatiles were evaporated in vacuo. The resultant oil was triturated with Et₂O to give a solid that was filtered and dried in vacuo affording intermediate 1 as a red solid (2.8 g, quantitative).

Intermediate 1 (2.69 g) was separated into enantiomers via chiral SFC (Stationary phase: Chiralpak AD-H 5 μm 250×30 mm, mobile phase: 80% CO₂, 20% ⁱPrOH+0.3% NH₄OH) yielding intermediate 1a (1.08 g) and intermediate 1b (1.15 g).

### Preparation of Intermediate 2

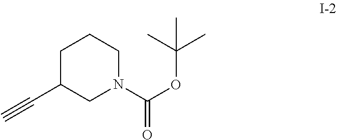

Dimethyl (1-diazo-2-oxopropyl)phosphonate (CAS: 90965-06-3; 36 mL, 10% solution in acetonitrile) in MeOH (40 mL) was added dropwise to a stirred mixture of 1-boc-3-piperidinecarboxaldehyde (2 g, 9.38 mmol) and K₂CO₃ (5.18 g, 37.5 mmol) in MeOH (80 mL) and the resulting mixture was stirred at rt for 16 h. The reaction mixture was concentrated in vacuo and the residue was dissolved in DCM and washed with. NaHCO₃ (aq. sat. soltn). The organic layer was separated and the aqueous phase was extracted with DCM. The combined organic extracts were dried over Na₂SO₄, filtered, and the filtrate was concentrated in vacuo yielding intermediate 2 that was used without further purification (1.96 g, 90% yield).

### Preparation of Intermediate 3

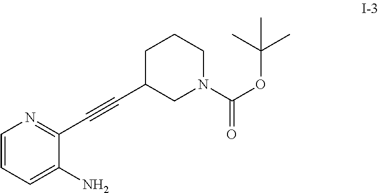 [SMILES: CC(C)(C)OC(=O)N1CCCC(C#Cc2ncccc2N)C1]

A solution of intermediate 2 (1.8 g, 8.6 mmol), 3-amino-2-bromopyridine (1.19 g, 6.88 mmol), copper (I) iodide (181 mg, 0.258 mmol) and PdCl₂(PPh₃)₂ (49 mg, 0.258 mmol) in DMF (10 mL) was purged by bubbling nitrogen and treated with triethylamine (3.59 mL, 25.8 mmol). The mixture was stirred at 100° C. for 1 h. Then it was cooled to rt and poured in EtOAc. The organic layer was separated, washed with water, brine, dried (Na₂SO₄), filtered and the filtrate concentrated in vacuo. The resultant dark yellow oil was purified by flash column chromatography (silica, EtOAc in heptane 0/100 to 80/20) and the desired fractions were concentrated in vacuo to yield intermediate 3 as a yellow solid (1.36 g, 66% yield).

### Preparation of Intermediate 4

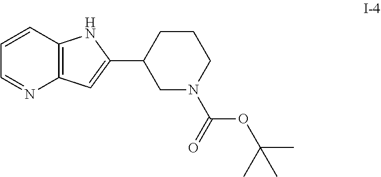 [SMILES: CC(C)(C)OC(=O)N1CCCC(c2cc3ncccc3[nH]2)C1]

Potassium tert-butoxide (1 g, 8.96 mmol) was added to a stirred solution of intermediate 3 (1.33 g, 4.48 mmol) in DMF (10 mL) at rt. The mixture was stirred at rt for 18 h and then it was poured in a mixture of water and EtOAc. The organic layer was separated, washed with water, brine, dried over Na₂SO₄, filtered and concentrated in vacuo. The resultant dark yellow oil was purified by flash column chromatography (silica; EtOAc in heptane 0/100 to 80/20) and the desired fractions were concentrated in vacuo to yield intermediate 4 as a white solid (1.045 g, 77% yield).

### Preparation of Intermediate 5

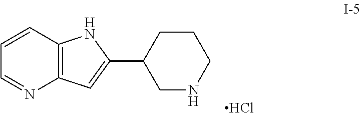 [SMILES: c1cnc2cc(C3CCCNC3)[nH]c2c1 ; Cl]

A mixture of intermediate 4 (3 g, 9.95 mmol) and HCl (80 mL; 4 M in 1,4-dioxane) was stirred at rt for 2 h. The volatiles were evaporated in vacuo affording intermediate 5 (2.45 g, 95% yield).

### Preparation of Intermediate 6

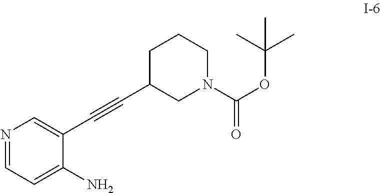 [SMILES: CC(C)(C)OC(=O)N1CCCC(C#Cc2cnccc2N)C1]

A solution of intermediate 2 (1.12 g, 5.34 mmol), 4-amino-3-bromopyridine (0.924 g, 5.34 mmol), copper (I) iodide (102 mg, 0.534 mmol), triethylamine (3.59 mL, 25.8 mmol) and PdCl₂(PPh₃)₂ (187 mg, 0.267 mmol) in DMF (9.24 mL) was purged by bubbling nitrogen. The mixture was stirred at 100° C. overnight under N₂. The mixture was cooled to rt and filtered through Celite® and the filtrate was kept. The Celite® plug was washed with EtOAc and the combined filtrates were washed with brine (3×), dried over MgSO₄, filtered and the volatiles were evaporated in vacuo. The crude material was purified on a 24 g SiO₂ gold column (elution with 0-5% MeOH containing 2M NH₃ over 7 min). The desired fractions were concentrated in vacuo to yield intermediate 6 as a light yellow solid (330 mg, 20% yield).

### Preparation of Intermediate 7

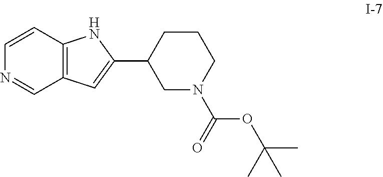 [SMILES: CC(C)(C)OC(=O)N1CCCC(c2cc3cnccc3[nH]2)C1]

Potassium tert-butoxide (3.24 mL, 1M solution in THF) was added to a stirred solution of intermediate 6 (0.32 g, 1.06 mmol) in N-methylpyrrolidone (3.24 mL) at rt. The mixture was heated and stirred at 70° C. overnight. The mixture diluted with water and EtOAc. The organic layer was separated, washed with brine, dried over Na₂SO₄, filtered and concentrated in vacuo. The resultant residue was purified on a 12 g SiO₂ gold column (elution with 0-5% MeOH in DCM containing 2M NH₃ over 8 min). The desired fractions were concentrated in vacuo to yield intermediate 7 as yellow foam (279 mg, 87% yield).

### Preparation of Intermediate 8

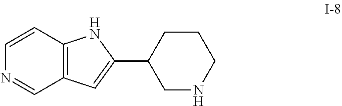 [SMILES: c1cc2[nH]c(C3CCCNC3)cc2cn1]

Trifluoroacetic acid (0.7 mL, 9.11 mmol) was added to a solution of intermediate 7 (279 mg, 0.926 mmol) in DCM (4.65 mL) at rt and the mixture was further stirred for 14 h. The volatiles were evaporated in vacuo to give the bistrifluoroacetate salt of intermediate 8 as yellow oil (424 mg, 107% yield).

### Preparation of Intermediate 9

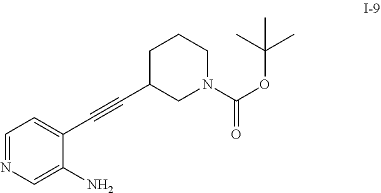 [SMILES: CC(C)(C)OC(=O)N1CCCC(C#Cc2ccncc2N)C1]

A solution of intermediate 2 (227 mg, 1.08 mmol), 3-amino-4-bromopyridine (150 mg, 0.867 mmol), copper (I) iodide (6.19 mg, 0.0325 mmol) and PdCl₂(PPh₃)₂ (22.8 mg, 0.0325 mmol) in DMF (2.12 mL) was degassed with a stream of N₂ and triethylamine (0.452 mL, 3.25 mmol) was added at rt. The reaction mixture was heated and stirred at 100° C. for 1 h. The mixture was cooled to rt and poured in EtOAc. The organic layer was separated and washed with water and brine, dried over Na₂SO₄, filtered and the volatiles were evaporated in vacuo. The resultant dark yellow oil was purified by flash chromatography (silica; MeOH in DCM, 0/100 to 5/95). The desired fractions were concentrated in vacuo to yield intermediate 9 as yellow solid (70 mg, 27% yield).

### Preparation of Intermediate 10

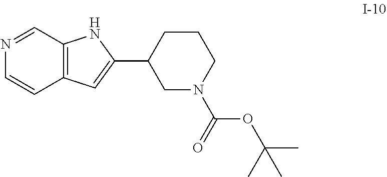 [SMILES: CC(C)(C)OC(=O)N1CCCC(c2cc3ccncc3[nH]2)C1]

Potassium tert-butoxide (141 mg, 1.26 mmol) was added to a stirred solution of intermediate 9 (190 mg, 0.63 mmol) in DMF (3.8 mL) at rt. The mixture was stirred at rt for 18 h. The mixture was diluted with water and EtOAc. The organic layer was separated, washed with water and brine, dried over Na₂SO₄, filtered and concentrated in vacuo. The resulting residue was purified by flash chromatography (silica; MeOH in DCM, 0/100 to 5/95). The desired fractions were concentrated in vacuo to yield intermediate 10 as a light yellow solid (135 mg, 71% yield).

### Preparation of Intermediate 11

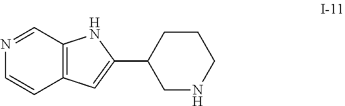 [SMILES: c1cc2cc(C3CCCNC3)[nH]c2cn1]

HCl (1.12 mL; 4 M in 1,4-dioxane) was added to a solution of intermediate 10 (135 mg, 0.448 mmol) in 1,4-dioxane (1.127 mL) at rt and the mixture was further stirred for 1 h. The volatiles were evaporated in vacuo and the residue thus obtained was taken up in MeOH and the solution was passed through an Isolute SCX-2 cartridge. The liquid was discarded and the product was then eluted from the cartridge with a solution of ammonia in MeOH. This later solution was evaporated in vacuo to give intermediate 11 as a white solid. (65 mg, 72% yield).

### Preparation of Intermediate 12

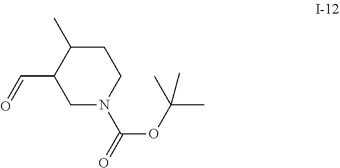 [SMILES: CC1CCN(C(=O)OC(C)(C)C)CC1C=O]

A mixture of piperidinecarboxylic acid, 3-(hydroxymethyl)-4-methyl-1,1-dimethylethyl ester (CAS: 1201187-00-9; 13 g, 56.7 mmol), Dess-Martin periodinane (CAS: 87413-09-0; 28.8 g, 68 mmol) in DCM (0.5 L) was stirred at rt for 16 h. The volatiles were evaporated in vacuo and water (100 mL) was added. The mixture was extracted with EtOAc (200 mL, 3×). The combined organic extracts were dried over Na₂SO₄, filtered and the filtrate was evaporated in vacuo to give crude intermediate 12 (14 g, 76% yield).

### Preparation of Intermediate 13

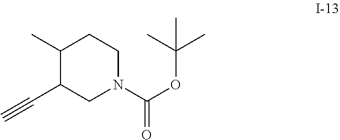 [SMILES: C#CC1CN(C(=O)OC(C)(C)C)CCC1C]

A mixture of dimethyl (1-diazo-2-oxopropyl)phosphonate (3.38 g, 17.6 mmol) intermediate 12 (2 g, 8.8 mmol) and K₂CO₃ (3.65 g, 26.4 mmol) in MeOH (15 mL) was stirred at rt for 12 h. The reaction mixture was filtered through Celite® and the filtrate was concentrated in vacuo. The resultant residue was purified by flash chromatography (silica; EtOAc in petroleum ether, 0/100 to 1/50). The desired fractions were concentrated in vacuo to yield intermediate 13 as colorless oil (1.5 g, 61% yield).

### Preparation of Intermediate 14

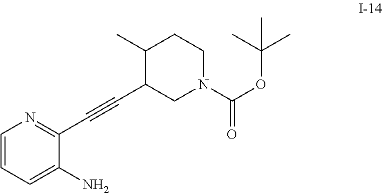 [SMILES: CC1CCN(C(=O)OC(C)(C)C)CC1C#Cc1ncccc1N]

A solution of intermediate 13 (9.5 g, 42.5 mmol), 3-amino-2-bromopyridine (11 g, 63.8 mmol), K₂CO₃ (14.7 g, 106.3 mmol) and Pd(PPh₃)₄ (2.45 g, 2.13 mmol) in acetonitrile (100 mL) was stirred at 90° C. for 12 h. The mixture was cooled to rt and the solvent was evaporated in vacuo. The organic layer was separated and washed with water and brine, dried over Na₂SO₄, filtered and the volatiles were evaporated in vacuo. The crude product was purified by flash chromatography (silica; EtOAc in petroleum ether, 1/15 to 1/4). The desired fractions were concentrated in vacuo to yield intermediate 14 as yellow solid (6 g, 43% yield).

### Preparation of Intermediate 15

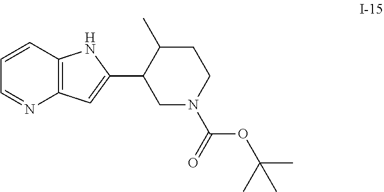 [SMILES: CC1CCN(C(=O)OC(C)(C)C)CC1c1cc2ncccc2[nH]1]

Sodium hydride (2.28 g, 57 mmol, 60% dispersion in mineral oil) was added to a stirred solution of intermediate 14 (6 g, 19 mmol) in DMF (50 mL) at rt. The mixture was stirred at 80° C. overnight. The mixture diluted with water (50 mL) and extracted with EtOAc (100 mL, 2×). The combined organic extracts were washed with brine (100 mL, 2×), dried over Na₂SO₄, filtered and concentrated in vacuo to yield crude intermediate 15 as a solid (3.2 g, 52% yield).

### Preparation of Intermediate 16

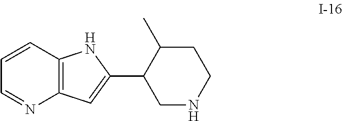 [SMILES: CC1CCNCC1c1cc2ncccc2[nH]1]

Intermediate 15 (318 mg, 0.992 mmol) was added to HCl (7 mL; 4 M in 1,4-dioxane) at rt and the mixture was further stirred at rt for 1 h. The volatiles were evaporated in vacuo to give the hydrochloric acid salt of intermediate 16 as a white solid (0.3 g, quantitative).

### Preparation of Intermediate 17

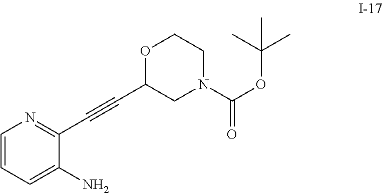 [SMILES: CC(C)(C)OC(=O)N1CCOC(C#Cc2ncccc2N)C1]

A solution of 4-morpholinecarboxylic acid, 2-ethynyl-, 1,1-dimethylethyl ester (CAS: 1416229-07-6; 5.5 g, 26 mmol), 3-amino-2-bromopyridine (6.75 g, 39 mmol), K₂CO₃ (9 g, 65 mmol) and Pd(PPh₃)₄ (1.50 g, 1.3 mmol) in acetonitrile (100 mL) was stirred at 90° C. for 12 h. The mixture was cooled to rt and the solvent was evaporated in vacuo. The crude product was purified by flash chromatography (silica; EtOAc in petroleum ether, 1/15 to 1/4). The desired fractions were concentrated in vacuo to yield intermediate 17 as yellow solid (4.8 g, 42% yield).

### Preparation of Intermediate 18

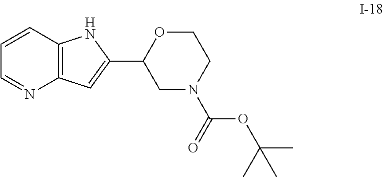 [SMILES: CC(C)(C)OC(=O)N1CCOC(c2cc3ncccc3[nH]2)C1]

Sodium hydride (47.5 mg, 1.97 mmol, 60% in mineral oil) was added to a stirred solution of intermediate 17 (300 mg, 0.99 mmol) in DMF (10 mL) at rt. The mixture was stirred at 80° C. overnight. The mixture diluted with water (50 mL) and extracted with EtOAc (50 mL, 2×). The combined organic extracts were washed with brine (50 mL, 2×), dried over Na₂SO₄, filtered and concentrated in vacuo. The crude product was purified by flash chromatography (silica; EtOAc in petroleum ether, 1/20 to 1/1). The desired fractions were concentrated in vacuo to yield crude intermediate 18 as a white solid (150 mg, 39% yield).

### Preparation of Intermediate 19

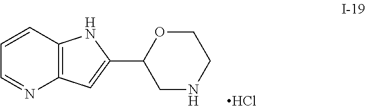 [SMILES: c1cnc2cc(C3CNCCO3)[nH]c2c1 ; Cl]

Intermediate 18 (2.5 g, 8.24 mmol) was added to HCl (50 mL; 4 M in 1,4-dioxane) at rt and the mixture was further stirred at rt for 1 h. The volatiles were evaporated in vacuo and water (20 mL) was added. The resulting mixture was washed with EtOAc (20 mL, 3×) and the resulting aqueous phase was evaporated under vacuum to give the hydrochloric acid salt of intermediate 19 as a solid (1.5 g, 75% yield).

### Preparation of Intermediate 20

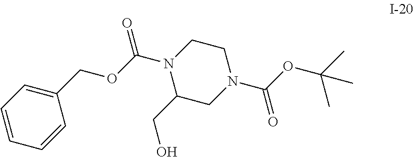 [SMILES: CC(C)(C)OC(=O)N1CCN(C(=O)OCc2ccccc2)C(CO)C1]

Benzyl chloroformate (189 mg, 1.12 mmol) was added to a stirred solution of 1-piperazinecarboxylic acid, 3-(hydroxymethyl)-, 1,1-dimethylethyl ester (CAS: 301673-16-5; 200 mg, 0.92 mmol) in THF (3 mL) and NaHCO₃(3 mL, aq. sat. soltn.). The resulting mixture was stirred at rt overnight. The mixture was then extracted with EtOAc (10 mL, 3×) and the combined organic extracts were dried over Na₂SO₄, filtered and concentrated in vacuo to yield intermediate 20 as colorless oil (280 mg, 37% yield).

### Preparation of Intermediate 21

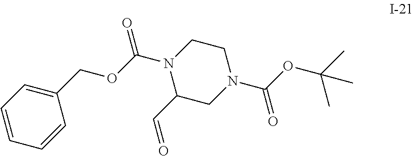 [SMILES: CC(C)(C)OC(=O)N1CCN(C(=O)OCc2ccccc2)C(C=O)C1]

To a stirred mixture of intermediate 20 (1 g, 2.85 mmol) in DCM (30 mL) at 0° C., Dess-Martin periodinane (CAS: 87413-09-0; 1.81 g, 4.28 mmol) was added. The mixture was allowed to warm to rt and then it was further stirred overnight. The volatiles were evaporated in vacuo to yield crude intermediate 21 (800 mg, 80% yield).

### Preparation of Intermediate 22

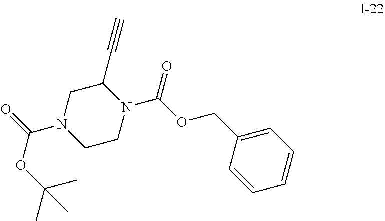 [SMILES: C#CC1CN(C(=O)OC(C)(C)C)CCN1C(=O)OCc1ccccc1]

Dimethyl (1-diazo-2-oxopropyl)phosphonate (7.83 g, 40.7 mmol) was added to a mixture of intermediate 21 (7.1 g, 20.37 mmol) and K₂CO₃ (8.45 g, 61.1 mmol) in MeOH (150 mL) at 0° C. The mixture was allowed to warm to rt and then it was further stirred overnight. The reaction mixture was filtered through Celite® and the filtrate was concentrated in vacuo to yield intermediate 22 as colorless oil (3.4 g, 48% yield).

### Preparation of Intermediate 23

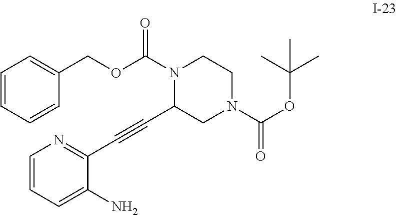 [SMILES: CC(C)(C)OC(=O)N1CCN(C(=O)OCc2ccccc2)C(C#Cc2ncccc2N)C1]

A solution of intermediate 22 (3.4 g, 9.87 mmol), 3-amino-2-bromopyridine (2.56 g, 14.8 mmol), K₂CO₃ (3.41 g, 24.7 mmol) and Pd(PPh₃)₄ (1.14 g, 0.99 mmol) in acetonitrile (80 mL) was stirred at 90° C. under N₂ overnight. The mixture was cooled to rt and the solvent was evaporated in vacuo. The crude product was purified by flash chromatography (silica; EtOAc in petroleum ether, 1/10 to 1/5). The desired fractions were concentrated in vacuo to yield intermediate 23 as brown solid (1.5 g, 32% yield).

### Preparation of Intermediate 24

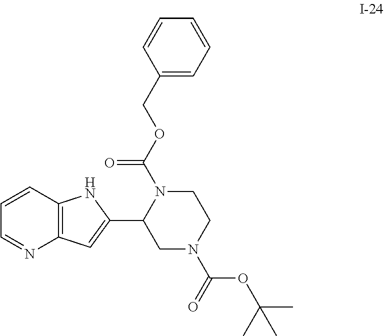 [SMILES: CC(C)(C)OC(=O)N1CCN(C(=O)OCc2ccccc2)C(c2cc3ncccc3[nH]2)C1]

Potassium tert-butoxide (458 mg, 4.1 mmol) was added to a stirred solution of intermediate 23 (890 mg, 2.04 mmol) in DMF (50 mL) at rt. The mixture was stirred at rt for 2 h. The mixture was diluted with water (20 mL) and extracted with EtOAc (30 mL, 3×). The combined organic extracts were dried over Na₂SO₄, filtered and concentrated in vacuo. The resultant residue was purified by flash chromatography (silica; EtOAc in petroleum ether, 1/3 to 1/1). The desired fractions were concentrated in vacuo to yield intermediate 24 as yellow solid (750 mg, 84% yield).

### Preparation of Intermediate 25

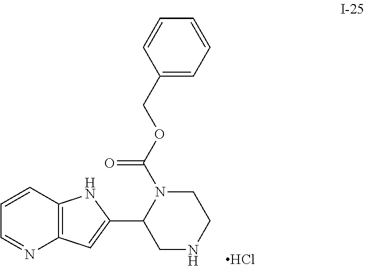 [SMILES: O=C(OCc1ccccc1)N1CCNCC1c1cc2ncccc2[nH]1 ; Cl]

Intermediate 24 (0.75 g, 1.72 mmol) was added to HCl (15 mL; 4 M in MeOH) at rt and the mixture was further stirred at rt for 1 h. The volatiles were evaporated in vacuo to give the hydrochloric acid salt of intermediate 25 as orange solid (1.5 g, 75% yield).

### Preparation of Intermediate 26

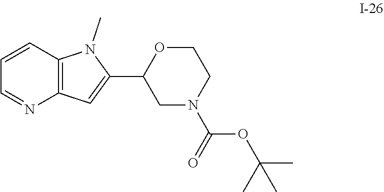 [SMILES: Cn1c(C2CN(C(=O)OC(C)(C)C)CCO2)cc2ncccc21]

Sodium hydride (122.1 mg, 3.05 mmol) was added to a stirred solution of intermediate 4 (0.92 g, 3.05 mmol) in DMF (27.6 mL) at 0° C. The mixture was allowed to warm to rt and then it was further stirred for 30 min. The reaction mixture was then cooled to 0° C. and methyl iodide (0.19 mL, 3.05 mmol) was added. The mixture was allowed to warm to rt it was further stirred for 1 h. Water and EtOAc were added. The organic layer was separated, washed with brine, dried over Na₂SO₄, filtered and concentrated in vacuo. The resultant dark yellow oil was purified by flash column chromatography (silica; MeOH in DCM 0/100 to 4/96) and the desired fractions were concentrated in vacuo to yield intermediate 26 as a yellow sticky solid (701 mg, 73% yield).

### Preparation of Intermediate 27

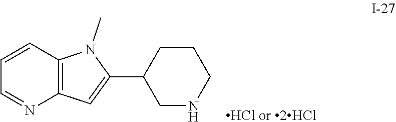

HCl (8.62 mL; 6 M in ⁱPrOH) was added to a stirred solution of intermediate 26 (5.44 g, 17.25 mmol) in MeOH (76.8 mL) at rt and the mixture was further stirred at rt for 48 h. The volatiles were evaporated in vacuo to give the hydrochloric acid salt of intermediate 27 as yellow solid (4.85 g, 97% yield).

### Preparation of Intermediate 28

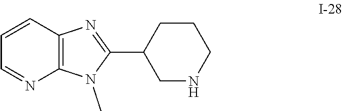 [SMILES: Cn1c(C2CCCNC2)nc2cccnc21]

A mixture of 3-amino-2-(methylamino)pyridine (265 mg, 2.15 mmol), nipecotic acid (CAS: 498-95-3; 281 mg, 2.18 mmol) and polyphosphoric acid (0.5 mL) was stirred at 180° C. for 72 h. Then the mixture was cooled to rt. Water was added and the mixture was stirred at 50° C. until it becomes homogeneous. This mixture was then cooled to room temperature and aqueous NaOH (3N) was added until pH=8 was reached. The volatiles were evaporated in vacuo, MeOH was added and the resulting mixture was filtered through a Celite® pad. The filtrate was collected and volatiles were evaporated in vacuo affording intermediate 28 as black oil (465 mg, quantitative).

### Preparation of Intermediate 29

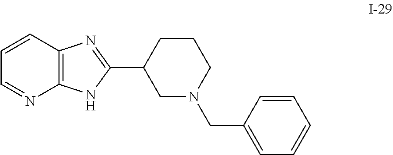 [SMILES: c1ccc(CN2CCCC(c3nc4cccnc4[nH]3)C2)cc1]

A mixture of 2,3-pyridinediamine (321 mg, 2.95 mmol), ethyl 1-benzyl-3-piperidinecarboxylate (CAS: 72551-53-2; 850 mg, 2.98 mmol) and polyphosphoric acid (5 mL) was stirred at 180° C. for 16 h. Then the mixture was cooled to rt. Water was added and the mixture was stirred at 50° C. until it became homogeneous. This mixture was then cooled to room temperature and aqueous NaOH (3N) was added until pH=8 was reached. Then EtOAc was added and the organic layer was separated, dried over Na₂SO₄, filtered and the filtrate was evaporated in vacuo. The resultant oil was purified by flash column chromatography (silica; 7M solution of ammonia in MeOH in EtOAc 0/100 to 10/90). The desired fractions were concentrated in vacuo to yield intermediate 29 as yellow oil (430 mg, 50% yield).

### Preparation of Intermediate 30

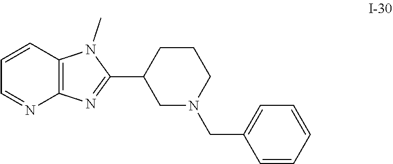 [SMILES: Cn1c(C2CCCN(Cc3ccccc3)C2)nc2ncccc21]

Sodium hydride (16.4 mg, 0.41 mmol, 60% in mineral oil) was added to a stirred solution of intermediate 29 (100 mg, 0.342 mmol) in THF (1.28 mL) at 0° C. under N₂ and the mixture further stirred at 0° C. for 1 h. Then methyl iodide (21.29 μL, 0.342 mmol) was added and the mixture was further stirred at 0° C. for 2 h. Water and EtOAc were added. The organic layer was separated, dried over Na₂SO₄, filtered and concentrated in vacuo. The resultant dark yellow oil was purified by flash column chromatography (silica; 7M solution of ammonia in MeOH in EtOAc, 0/100 to 50/50) and the desired fractions were concentrated in vacuo to yield intermediate 30 as yellow oil (66 mg, 63% yield).

### Preparation of Intermediate 31

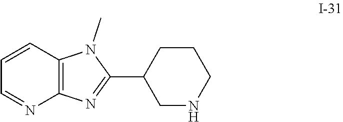 [SMILES: Cn1c(C2CCCNC2)nc2ncccc21]

Pd/C (10%) (41.67 mg, 0.039 mmol) was added to a stirred solution of intermediate 30 (60 mg, 0.342 mmol), ammonium formate (61.73 mg, 0.98 mmol) in MeOH (1.27 mL) at rt and then the mixture was stirred at 90° C. for 1 h into a sealed tube. MeOH was added and the mixture was filtered through a Celite® pad. The filtrate was concentrated in vacuo to yield intermediate 31 as colorless oil (30 mg, 71% yield).

### Preparation of Intermediate 32

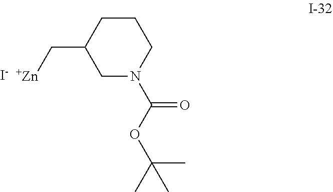

A solution of 3-iodomethylpiperidine-1-carboxylic acid tert-butyl ester (CAS: 253177-03-6; 1 g, 3.07 mmol) and LiCl (6.15 mL, 3.07 mmol, 0.5 M solution in THF) was pumped through a column containing activated Zn (12.3 g, 188.1 mmol) at 40° C. with flow of 0.5 mL/min. The outcome solution was collected under N₂ atmosphere to yield intermediate 32 as a clear solution that was used without any further manipulation.

For the above reaction Zn was activated as follows: A solution of TMSCl (2.2 mL) and 1-bromo-2-choroethane (0.5 mL) in THF (10 mL) was passed through the column containing Zn at a flow of 1 mL/min.

### Preparation of Intermediate 33

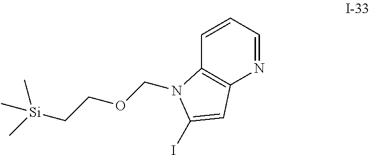 [SMILES: C[Si](C)(C)CCOCn1c(I)cc2ncccc21]

n-Buthyl lithium (2.51 mL, 4.02 mmol, 1.6 M in hexane) was added dropwise to a solution of 1-[[2-(trimethylsilyl)ethoxy]methyl]-1H-pyrrolo[3,2-b]pyridine (CAS: 1286777-45-4; 1 g, 4.03 mmol) in THF (12 mL) at −78° C. under N₂ atmosphere. The mixture was further stirred at −78° C. for 30 min. Then a solution of I₂ (1.23 g, 4.43 mmol) in THF (10 mL) was added at −78° C. and the reaction mixture was further stirred for 10 min. The reaction was then allowed to warm to rt and 10% aqueous Na₂SO₃ was added. The mixture was extracted with EtOAc. The organic layer was separated, dried over Na₂SO₄, filtered and the volatiles were evaporated in vacuo. The resultant residue was purified by flash column chromatography (silica; EtOAc in DCM 0/100 to 30/70) and the desired fractions were concentrated in vacuo to yield intermediate 33 as yellow oil (0.91 g, 60% yield).

### Preparation of Intermediate 34

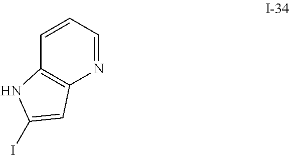

Trifluoroacetic acid (2 mL, 26.13 mmol) was added to a stirred solution of intermediate 33 (400 mg, 1.07 mmol) in DCM (2 mL) at rt and the resulting mixture was stirred for 3 h. The volatiles were evaporated in vacuo and the residue thus obtained was taken up in DCM and basified with NaHCO₃(aq. sat. soltn.). The solid was filtered, washed with Et₂O and then dried to yield intermediate 34 as an off-white solid (248 mg, 95%).

### Preparation of Intermediate 35

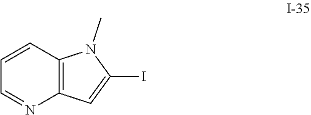

Sodium hydride (61 mg, 1.52 mmol, 60% dispersion in mineral oil) was added portionwise to a stirred solution of intermediate 34 (248 mg, 1.02 mmol) in DMF (2 mL) at 0° C. and the mixture further stirred at 0° C. for 10 min. Then methyl iodide (82.25 μL, 1.32 mmol) was added and the mixture was allowed to warm to rt and further stirred at rt for 1 h. NH₄Cl (aq. sat. soltn.) and EtOAc were added. The organic layer was separated, dried over Na₂SO₄, filtered and concentrated in vacuo. The residue thus obtained was purified by flash column chromatography (silica; EtOAc in DCM, 0/100 to 100/0) and the desired fractions were concentrated in vacuo to yield intermediate 35 as an off-white solid (131 mg, 50% yield).

### Preparation of Intermediate 36

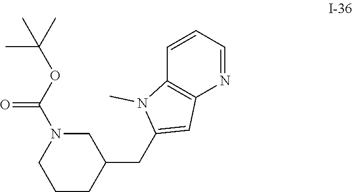 [SMILES: Cn1c(CC2CCCN(C(=O)OC(C)(C)C)C2)cc2ncccc21]

Intermediate 32 (0.378 mmol, 0.27 M in THF) was added to a mixture of intermediate 35 (65 mg, 0.252 mmol), Pd (OAc)₂ (2.827 mg, 0.0126 mmol) and 2-dicyclohexylphosphino-2′,6′-di-iso-propoxy-1,1′-biphenyl (also known as RuPhos) (CAS: 787618-22-8; 11.75 mg, 0.0252 mmol). The resulting mixture was stirred at rt for 20 min and then it was heated at 50° C. and further stirred for 30 min. 10% aqueous NH₄Cl was added and the mixture was extracted with EtOAc. The organic layer was separated, dried over Na₂SO₄, filtered and concentrated in vacuo. The residue thus obtained was purified by flash column chromatography (silica; EtOAc in DCM, 0/100 to 100/0) and the desired fractions were concentrated in vacuo to yield intermediate 36 as yellow oil (55 mg, 66% yield).

### Preparation of Intermediate 37

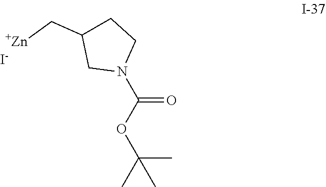

A solution of 3-iodomethylpyrrolidine-1-carboxylic acid tert-butyl ester (CAS: 479622-36-1; 0.93 g, 3 mmol) in THF (6 mL) was pumped through a column containing activated Zn (12 g, 183.5 mmol) at 40° C. with a flow of 0.5 mL/min. The outcome solution was collected under N₂ atmosphere to yield intermediate 37 as a clear solution that was used without any further manipulation.

For the above reaction Zn was activated as follows: A solution of TMSCl (0.75 mL) and 1-bromo-2-choroethane (0.3 mL) in THF (10 mL) was passed through the column containing Zn at 40° C. with a flow of 1 mL/min.

### Preparation of Intermediate 38

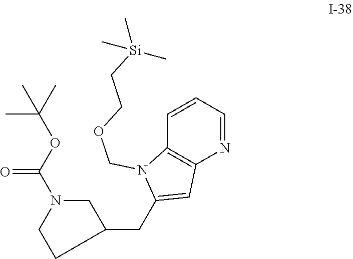 [SMILES: CC(C)(C)OC(=O)N1CCC(Cc2cc3ncccc3n2COCC[Si](C)(C)C)C1]

Intermediate 37 (1.202 mmol, 0.21 M in THF) was added to a mixture of intermediate 33 (300 mg, 0.801 mmol), Pd(OAc)₂ (9 mg, 0.0401 mmol) and 2-dicyclohexylphosphino-2′,6′-dimethoxybiphenyl (also known as SPhos) (CAS: 657408-07-6; 32.9 mg, 0.0801 mmol). The resulting mixture was heated at 50° C. and further stirred for 1 h. 10% aqueous NH₄Cl was added and the mixture was extracted with EtOAc. The organic layer was separated, dried over Na₂SO₄, filtered and concentrated in vacuo. The residue thus obtained was purified by flash column chromatography (silica; EtOAc in DCM, 0/100 to 100/0) and the desired fractions were concentrated in vacuo to yield intermediate 38 as yellow oil (426 mg, 75% yield).

### Preparation of Intermediate 39

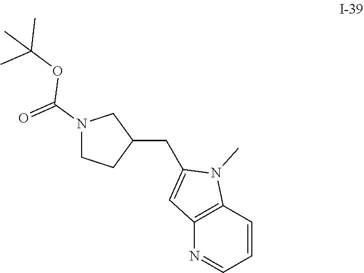 [SMILES: Cn1c(CC2CCN(C(=O)OC(C)(C)C)C2)cc2ncccc21]

Intermediate 37 (0.378 mmol, 0.23 M in THF) was added to a mixture of intermediate 35 (65 mg, 0.252 mmol), Pd(OAc)₂ (2.827 mg, 0.0126 mmol) and 2-dicyclohexylphosphino-2′,6′-di-iso-propoxy-1,1′-biphenyl (also known as RuPhos) (CAS: 787618-22-8; 11.75 mg, 0.0252 mmol). The resulting mixture was stirred at rt for 1 h. 10% aqueous NH₄Cl was added and the mixture was extracted with EtOAc. The organic layer was separated, dried over Na₂SO₄, filtered and concentrated in vacuo. The residue thus obtained was purified by flash column chromatography (silica; EtOAc in DCM, 0/100 to 100/0) and the desired fractions were concentrated in vacuo to yield intermediate 39 as yellow oil that solidified upon standing (63 mg, 79% yield).

### Preparation of Intermediate 40

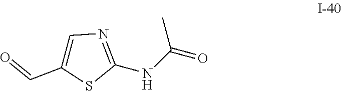

Acetyl choride (6 mL, 84.38 mmol) was added to a solution of 2-amino-5-formylthiazole (10 g, 78 mmol) and diisopropylamine (45 mL, 261.1 mmol) in DCM (100 mL) at 0° C. The resulting mixture was allowed to warm to rt and further stirred at rt for 17 h. NH₄C₁ (aq. sat. soltn.) was added and the mixture was extracted with EtOAc. The organic layer was separated, dried over MgSO₄, filtered and concentrated in vacuo. The residue thus obtained was purified by flash column chromatography (silica; dry load, EtOAc in DCM 0/100 to 50/50) and the desired fractions were concentrated in vacuo to yield intermediate 40 as yellow solid (8.6 g, 65% yield).

### Preparation of Intermediate 41

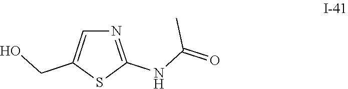

Lithium triethylborohydride (2.8 mL, 2.8 mmol; 1M solution in THF) was added to a solution of intermediate 40 (200 mg, 0.93 mmol) in THF (4.6 mL) cooled at −78° C. The mixture was allowed to warm to rt and then further stirred at rt for 16 h. Water and EtOAc were added and the organic phase was separated and discarded. The aqueous phase was evaporated to dryness and the resulting solid was washed with water, filtered, dried and purified by reverse phase HPLC (Stationary phase: C₁₈ X Bridge 30×100 mm 5 um), mobile phase: gradient from 90% 0.1% NH₄CO₃H/NH₄OH pH 9 solution in water, 10% CH₃CN to 0% 0.1% NH₄CO₃H/NH₄OH pH 9 solution in water, 100% CH₃CN). The desired fractions were concentrated in vacuo to yield intermediate 41 as a white solid (50 mg, 31% yield).

### Preparation of Intermediate 42

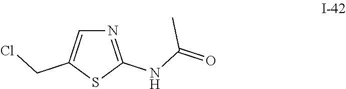

Sulfonyl chloride (0.042 mL, 0.51 mmol) was added to a solution of intermediate 41 (100 mg, 0.48 mmol) in DCM (3.05 mL) at 0° C. The mixture was allowed to warm to rt and then further stirred at rt for 1 h. The volatiles were evaporated in vacuo affording intermediate 42 as yellow solid (98 mg, 91% yield).

### Preparation of Intermediate 43

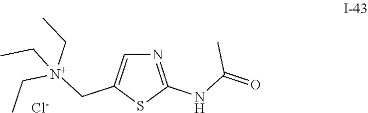 [SMILES: CC[N+](CC)(CC)Cc1cnc(NC(C)=O)s1 ; [Cl-]]

tert-Butyl dicarbonate (172 mg, 0.79 mmol) was added to a stirred solution of intermediate 42 (100 mg, 0.52 mmol) and triethylamine (0.145 mL, 1.05 mmol) in THF (4.27 mL) and the mixture was stirred at rt for 30 min. The resultant suspension was filtered and this solid was dried under vacuum affording intermediate 43 as yellow solid (36 mg, 27% yield).

### Preparation of Intermediate 44

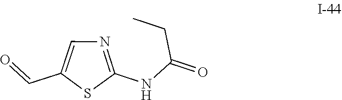

Propionic anhydride (0.45 mL, 3.51 mmol) was added to a suspension of 2-amino-5-formylthiazole (0.15 g, 1.17 mmol) in toluene (10 mL) at rt. The resulting mixture was heated at 110° C. for 5 h. Upon cooling to rt a solid precipitated and was filtered and dried to yield intermediate 44 as a pale yellow solid (215 mg, quantitative).

### Preparation of Intermediate 45

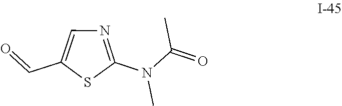

Acetic anhydride (0.55 mL, 5.83 mmol) was added to a suspension of 2-(methylamino)-5-thiazolecarboxaldehyde (CAS: 1263210-20-3; 0.166 g, 1.17 mmol) in toluene (6 mL) at rt. The reaction mixture was diluted with NaHCO₃(aq. sat. soltn.) and EtOAc. The organic layer was separated, dried over MgSO₄, filtered and the filtrate was evaporated in vacuo. The residue thus obtained was purified by flash column chromatography (silica; MeOH in DCM 1/99) and the desired fractions were concentrated in vacuo to yield intermediate 45 as a brown solid (90 mg, 41% yield).

### Preparation of Intermediate 46

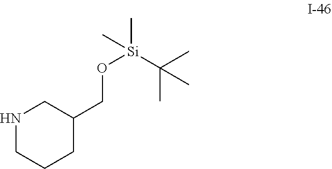

tert-Butyldimethylsilylchloride (2.88 g, 19.1 mmol) was added portion wise to a stirred solution of 3-piperidinemethanol (CAS: 4606-65-9; 2 g, 17.36 mmol) and diisopropylethylamine (6.05 mL, 34.73 mmol) in DCM (55 mL) under N₂ atmosphere at 0° C. The reaction mixture was allowed to warm to rt and further stirred at rt for 18 h. The mixture was diluted with NaHCO₃(aq. sat. soltn.) and extracted with DCM. The organic layer was separated, dried over MgSO₄, filtered and the filtrate was evaporated in vacuo. The residue thus obtained was purified by flash column chromatography (silica; EtOAc in heptane from 5/100 to 30/70) and the desired fractions were concentrated in vacuo to yield intermediate 46 (3.87 g, 92% yield).

### Preparation of Intermediate 47

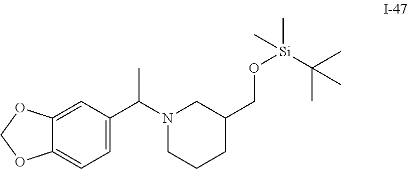 [SMILES: CC(c1ccc2c(c1)OCO2)N1CCCC(CO[Si](C)(C)C(C)(C)C)C1]

Piperonal (CAS: 120-57-0; 981.5 mg, 6.54 mmol) and Ti(O′Pr)₄ (1.94 mL, 6.54 mmol) were added to a solution of intermediate 46 (1 g, 4.36 mmol) in anhydrous DCM (13.48 mL) under N₂ atmosphere at rt. The reaction mixture was stirred at rt for 18 h. Then the reaction was cooled to 0° C. and methylmagnesium bromide (15.56 mL, 1.4 M solution in THF) was added dropwise followed by anhydrous THF (13.47 mL). The mixture was stirred at 0° C. for 5 min and then it was allowed to warm to rt and further stirred at rt for 3 h. NH₄Cl (aq. sat. soltn.) and DCM were added. The organic layer was separated, dried over MgSO₄, filtered and the filtrate was evaporated in vacuo. The residue thus obtained was purified by flash column chromatography (silica; EtOAc in heptane from 5/100 to 30/70) and the desired fractions were concentrated in vacuo to yield intermediate 47 (1.25 g, 76% yield).

### Preparation of Intermediate 48

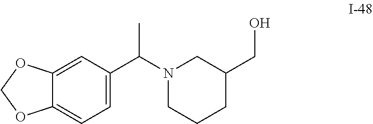 [SMILES: CC(c1ccc2c(c1)OCO2)N1CCCC(CO)C1]

Tetrabutylammonium fluoride (1.85 g, 6.87 mmol) was added to a solution of intermediate 47 (1.11 g, 2.94 mmol) in THF (4 mL) at rt and the reaction mixture was stirred at rt overnight. The mixture was diluted with water and extracted with EtOAc. The organic layer was separated, dried over MgSO₄, filtered and the filtrate was evaporated in vacuo. The resultant residue was purified by flash chromatography (silica; EtOAc in heptane, 0/100 to 100/0). The desired fractions were concentrated in vacuo to yield intermediate 48 (0.65 g, 80% yield) as colorless oil.

### Preparation of Intermediate 49

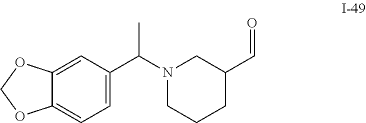 [SMILES: CC(c1ccc2c(c1)OCO2)N1CCCC(C=O)C1]

Oxalyl chloride (0.313 mL, 3.70 mmol) was added dropwise to a solution of DMSO (0.265 mL) in DCM (33 mL) at −78° C. The reaction mixture was further stirred at −78° C. for 20 min. Then a solution of intermediate 48 (0.65 g, 2.47 mmol) in DCM (10 mL) was added dropwise at −78° C. and the reaction mixture was further stirred at −78° C. for 1 h. Triethylamine (2.05 mL, 14.8 mmol) was added dropwise at −78° C. and the reaction mixture was allowed to warm to rt. The mixture was diluted with water. The organic layer was separated, dried over MgSO₄, filtered and the filtrate was evaporated in vacuo. The residue thus obtained was purified by flash column chromatography (silica; EtOAc in heptane from 0/100 to 30/70) and the desired fractions were concentrated in vacuo to yield intermediate 49 as colorless oil (0.49 g, 75% yield).

### Preparation of Intermediate 50

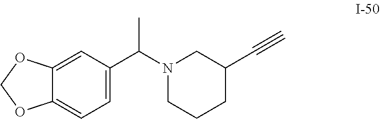 [SMILES: C#CC1CCCN(C(C)c2ccc3c(c2)OCO3)C1]

Dimethyl (1-diazo-2-oxopropyl)phosphonate (0.36 g, 1.87 mmol) in MeOH (5 mL) was added dropwise to a solution of intermediate 49 (0.49 g, 1.87 mmol) and K₂CO₃ (0.52 g, 3.75 mmol) in MeOH (5 mL) and the mixture was stirred at rt for 3 h. Then the volatiles were evaporated in vacuo. The residue was taken up in EtOAc and NaHCO₃(aq. sat. soltn.). The organic layer was separated, dried over MgSO₄, filtered and the filtrate was evaporated in vacuo. The resultant residue was purified by flash chromatography (silica; EtOAc in heptane, 0/100 to 30/70). The desired fractions were concentrated in vacuo to yield intermediate 50 (0.36 g, 71% yield).

### Preparation of Intermediate 51

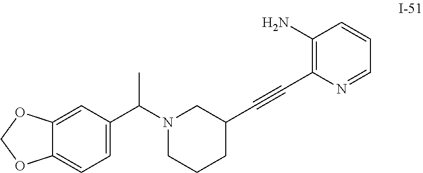 [SMILES: CC(c1ccc2c(c1)OCO2)N1CCCC(C#Cc2ncccc2N)C1]

A solution 3-amino-2-bromopyridine (232 mg, 1.34 mmol) and triethylamine (2.81 mL, 20.1 mmol) in anhydrous DMF (5 mL) was degassed with a stream of argon for 10 min. Then copper (I) iodide (12.78 mg, 0.067 mmol), PdCl₂(PPh₃)₂ (22.27 mg, 0.04 mmol) an intermediate 50 (0.38 g, 1.48 mmol) were added at rt. The reaction mixture was heated and stirred at 80° C. for 1 h. The mixture was cooled to rt and diluted with EtOAc and brine. The organic layer was separated, dried over Na₂SO₄, filtered and the volatiles were evaporated in vacuo. The resultant residue was purified by flash chromatography (silica; EtOAc in heptane, 0/100 to 50/50). The desired fractions were concentrated in vacuo to yield intermediate 51 as yellow oil (400 mg, 84% yield).

### Preparation of Intermediate 52

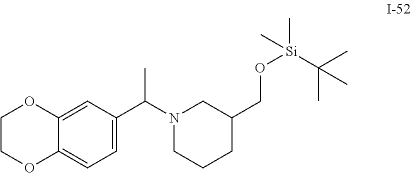 [SMILES: CC(c1ccc2c(c1)OCCO2)N1CCCC(CO[Si](C)(C)C(C)(C)C)C1]

1,4-Benzodioxan-6-carboxaldehyde (CAS: 29668-44-8; 1.27 g, 7.71 mmol) and Ti(O'Pr)₄ (2.28 mL, 7.71 mmol) were added to a solution of intermediate 46 (1.18 g, 5.14 mmol) in anhydrous DCM (15.81 mL) under N₂ atmosphere at rt. The reaction mixture was stirred at rt for 18 h. Then the reaction was cooled to 0° C. and methylmagnesium bromide (15.56 mL, 1.4 M solution in THF) was added dropwise followed by anhydrous THF (15.9 mL). The mixture was stirred at 0° C. for 5 min and then it was allowed to warm to rt and further stirred at rt for 3 h. NH₄Cl (aq. sat. soltn.) and DCM were added. The organic layer was separated, dried over MgSO₄, filtered and the filtrate was evaporated in vacuo. The resultant residue was purified by flash chromatography (silica; EtOAc in heptane, 10/100 to 20/80). The desired fractions were concentrated in vacuo to yield intermediate 52 (1.6 g, 79% yield).

### Preparation of Intermediate 53

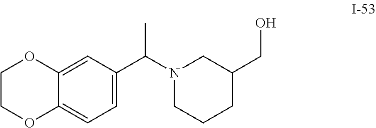 [SMILES: CC(c1ccc2c(c1)OCCO2)N1CCCC(CO)C1]

Tetrabutylammonium fluoride (3.29 g, 10.42 mmol) was added to a solution of intermediate 52 (2.04 g, 5.21 mmol) in THF (52 mL) at rt. The reaction mixture was stirred at rt for 2 h. The mixture was diluted with water and extracted with EtOAc. The organic layer was separated, dried over MgSO₄, filtered and the filtrate was evaporated in vacuo. The resultant residue was purified by flash chromatography (silica; EtOAc in heptane, 0/100 to 100/0). The desired fractions were concentrated in vacuo to yield intermediate 53 (0.604 g, 42% yield) as colorless oil.

### Preparation of Intermediate 54

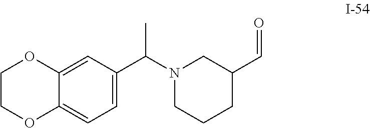 [SMILES: CC(c1ccc2c(c1)OCCO2)N1CCCC(C=O)C1]

Oxalyl chloride (0.274 mL, 3.24 mmol) was added dropwise to a solution of DMSO (0.233 mL) in DCM (25 mL) at −78° C. The reaction mixture was further stirred at −78° C. for 20 min. Then a solution of intermediate 53 (0.6 g, 2.16 mmol) in DCM (6 mL) was added dropwise at −78° C. and the reaction mixture was further stirred at −78° C. for 1 h. Triethylamine (1.8 mL, 13 mmol) was added dropwise at −78° C. and the reaction mixture was allowed to warm to rt under stirring for 90 min. The mixture was diluted with water. The organic layer was separated, dried over MgSO₄, filtered and the filtrate was evaporated in vacuo. The residue thus obtained was purified by flash column chromatography (silica; EtOAc in heptane from 0/100 to 30/70) and the desired fractions were concentrated in vacuo to yield intermediate 54 (0.397 g, 67% yield).

### Preparation of Intermediate 55

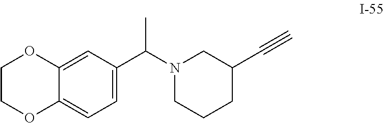 [SMILES: C#CC1CCCN(C(C)c2ccc3c(c2)OCCO3)C1]

Dimethyl (1-diazo-2-oxopropyl)phosphonate (0.277 g, 1.44 mmol) in MeOH (5 mL) was added dropwise to a solution of intermediate 54 (0.397 g, 1.44 mmol) and K₂CO₃ (0.399 g, 2.88 mmol) in MeOH (5 mL) and the mixture was stirred at rt for 3 h. Then the volatiles were evaporated in vacuo. The residue was taken up in EtOAc and NaHCO₃(aq. sat. soltn.). The organic layer was separated, dried over MgSO₄, filtered and the filtrate was evaporated in vacuo. The resultant residue was purified by flash chromatography (silica; EtOAc in heptane, 0/100 to 30/70). The desired fractions were concentrated in vacuo to yield intermediate 55 (0.27 g, 69% yield).

### Preparation of Intermediate 56

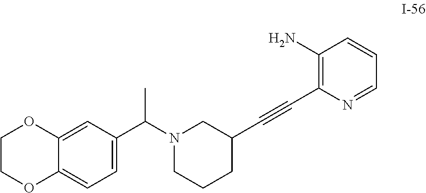 [SMILES: CC(c1ccc2c(c1)OCCO2)N1CCCC(C#Cc2ncccc2N)C1]

A solution 3-amino-2-bromopyridine (156 mg, 0.904 mmol) in anhydrous DMF (3 mL) was degassed with a stream of argon. Then copper (I) iodide (8.61 mg, 0.045 mmol), PdCl₂(PPh₃)₂ (19.04 mg, 0.027 mmol), intermediate 55 (0.27 g, 1 mmol) and triethylamine (1.89 mL, 13.6 mmol) were added at rt. The reaction mixture was heated and stirred at 80° C. for 90 min. The mixture was cooled to rt and diluted with EtOAc and brine. The organic layer was separated, dried over Na₂SO₄, filtered and the volatiles were evaporated in vacuo. The resultant residue was purified by flash chromatography (silica; EtOAc in heptane, 0/100 to 50/50). The desired fractions were concentrated in vacuo to yield intermediate 56 as yellow oil (250 mg, 76% yield).

### Preparation of Intermediate 57

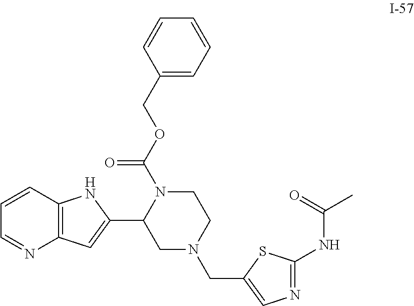 [SMILES: CC(=O)Nc1ncc(CN2CCN(C(=O)OCc3ccccc3)C(c3cc4ncccc4[nH]3)C2)s1]

To intermediate 25 (240 mg, 0.71 mmol) in DCM (8 mL) diisopropylethylamine (0.615 mL, 3.57 mmol) was added and the mixture was stirred at rt for 10 min. Then intermediate 40 (0.146 g, 0.86 mmol) was added and the resulting suspension was stirred at rt for 2 h. Sodium triacetoxyborohydride (333 mg; 1.57 mmol) was added and the reaction was stirred at rt for 5 h. Then sodium cyanoborohydride (99 mg, 1.57 mmol) followed by a solution of acetic acid (0.0204 mL, 0.36 mmol) in MeOH (2 mL) were added and the resulting mixture was stirred at rt for 5 h. Water was added and the organic layer was separated, dried over Na₂SO₄, filtered and the volatiles were evaporated in vacuo. The resulting residue was purified by flash chromatography (silica; MeOH in DCM, 0/100 to 5/95). The desired fractions were concentrated in vacuo to yield intermediate 57 as colorless oil (115 mg, 33% yield).

### Preparation of Intermediate 58

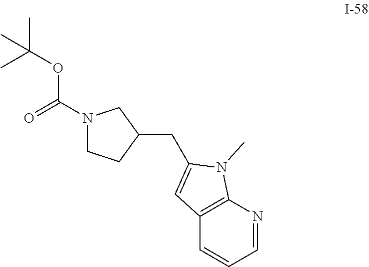 [SMILES: Cn1c(CC2CCN(C(=O)OC(C)(C)C)C2)cc2cccnc21]

Intermediate 37 (0.775 mmol, 0.34 M in THF) was added to a mixture of 2-iodo-1-methyl-1H-pyrrolo[2,3-b]pyridine (CAS: 1388711-09-8; 100 mg, 0.39 mmol), Pd(OAc)₂ (4.35 mg, 0.0194 mmol) and 2-dicyclohexylphosphino-2′,6′-diisopropoxy-1,1′-biphenyl (also known as RuPhos) (CAS: 787618-22-8; 18.1 mg, 0.039 mmol). The resulting mixture was stirred at rt for 1 h. 10% aqueous NH₄Cl was added and the mixture was extracted with EtOAc. The organic layer was separated, dried over Na₂SO₄, filtered and concentrated in vacuo. The residue thus obtained was purified by flash column chromatography (silica; EtOAc in heptane 0/100 to 100/0) and the desired fractions were concentrated in vacuo to yield intermediate 58 as yellow oil (80 mg, 65% yield).

### Preparation of Intermediate 59

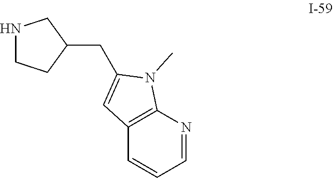 [SMILES: Cn1c(CC2CCNC2)cc2cccnc21]

Trifluoroacetic acid (0.47 mL, 6.2 mmol) was added to a solution of intermediate 58 (80 mg, 0.25 mmol) in DCM (0.47 mL). The resulting mixture was stirred at rt for 16 h. The solvent was evaporated in vacuo and the resulting solid was taken up in MeOH and purified by ion exchange chromatography (isolute SCX2 cartridge eluting with MeOH and 7N solution of NH₃ in MeOH). The desired fractions were concentrated in vacuo to yield intermediate 59 as pale brown solid (46 mg, 82% yield).

### Preparation of Intermediate 60

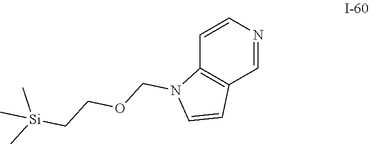 [SMILES: C[Si](C)(C)CCOCn1ccc2cnccc21]

5-azaindole (2 g, 17 mmol) was added portionwise to a suspension of sodium hydride (744 mg, 18.6 mmol, 60% dispersion in mineral oil) in DMF (40 mL) at 0° C. The mixture was stirred at rt for 10 min. Then the mixture was cooled to 0° C. and 2-(trimethylsilyl)ethoxymethyl chloride (18.6 mmol) was added. The reaction mixture was further stirred at rt for 16 h. NH₄Cl (aq. Sat. soltn.) was added and the mixture was extracted with EtOAc. The organic layer was separated, dried over Na₂SO₄, filtered and the volatiles were evaporated in vacuo. The resultant residue was purified by flash column chromatography (silica; EtOAc in heptane 0/100 to 50/50) and the desired fractions were concentrated in vacuo to yield intermediate 60 as brown oil (2.1 g, 50% yield).

### Preparation of Intermediate 61

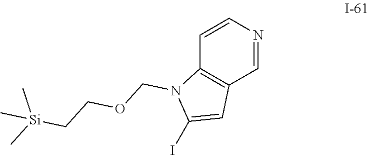 [SMILES: C[Si](C)(C)CCOCn1c(I)cc2cnccc21]

n-Butyl lithium (5.07 mL, 12.68 mmol, 2.5 M in hexane) was added dropwise to a stirred solution of intermediate 60 (2.1 g, 8.45 mmol) in THF (30 mL) at −40° C. under N₂ atmosphere. The mixture was further stirred at −40° C. for 1 h. Then a solution of 12 (5.36 g, 4.43 mmol) in THF (18 mL) was added at −40° C. and the reaction mixture was further stirred for 30 min. The reaction was then allowed to warm to rt and Na₂S₂O₃ (aq. sat. soltn) was added. The mixture was extracted with EtOAc. The organic layer was separated, dried over Na₂SO₄, filtered and the volatiles were evaporated in vacuo. The resultant residue was purified by flash column chromatography (silica; EtOAc in heptane, 0/100 to 30/70) and the desired fractions were concentrated in vacuo to yield intermediate 61 as a cream solid (1.03 g, 32% yield).

### Preparation of Intermediate 62

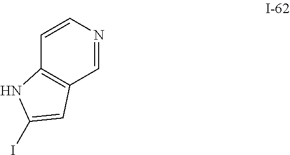

Trifluoroacetic acid (2 mL, 26.13 mmol) was added to a stirred solution of intermediate 61 (400 mg, 1.07 mmol) in DCM (2 mL) at rt and the resulting mixture was stirred for 16 h. The volatiles were evaporated in vacuo and the residue thus obtained was taken up in MeOH and purified by ion exchange chromatography (isolute SCX2 cartridge eluting with MeOH and 7N solution of NH₃ in MeOH). The desired fractions were concentrated in vacuo to yield intermediate 62 as pale brown solid (254 mg, 97% yield).

### Preparation of Intermediate 63

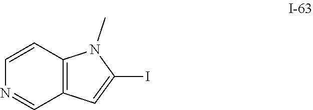

Intermediate 62 (254 mg, 1.04 mmol) was added portionwise to a suspension of sodium hydride (45.8 mg, 1.15 mmol, 60% dispersion in mineral oil) in DMF (2.46 mL) at 0° C. The mixture was stirred at rt for 30 min. Then the mixture was cooled to 0° C. and methyl iodide (71.28 μL, 1.45 mmol) was added and the mixture was allowed to warm to rt and further stirred at rt for 16 h. Brine and EtOAc were added. The organic layer was separated, dried over Na₂SO₄, filtered and concentrated in vacuo. The residue thus obtained was purified by flash column chromatography (silica; EtOAc in heptane 0/100 to 100/0) and the desired fractions were concentrated in vacuo to yield intermediate 63 as a pale brown solid (159 mg, 59% yield).

### Preparation of Intermediate 64

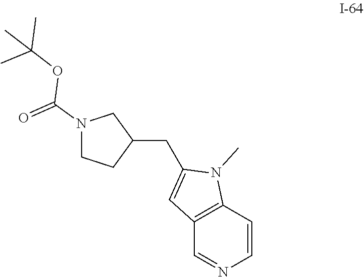 [SMILES: Cn1c(CC2CCN(C(=O)OC(C)(C)C)C2)cc2cnccc21]

Intermediate 37 (2.46 mmol, 0.34 M in THF) was added to a mixture of intermediate 63 (159 mg, 0.61 mmol), Pd (OAc)₂ (6.92 mg, 0.031 mmol) and 2-dicyclohexylphosphino-2′,6′-di-isopropoxy-1,1′-biphenyl (also known as RuPhos) (CAS: 787618-22-8; 28.75 mg, 0.061 mmol). The resulting mixture was stirred at rt for 18 h. 10% aqueous NH₄Cl was added and the mixture was extracted with EtOAc. The organic layer was separated, dried over Na₂SO₄, filtered and concentrated in vacuo. The resulting residue was triturated with Et₂O and filtered to yield intermediate 64 as a brown solid (119 mg, 61% yield; 81% pure) that was used without further purification

### Preparation of Intermediate 65

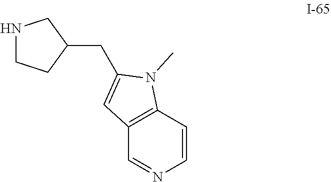 [SMILES: Cn1c(CC2CCNC2)cc2cnccc21]

Trifluoroacetic acid (0.71 mL, 9.23 mmol) was added to a solution of intermediate 64 (119 mg, 0.38 mmol) in DCM (0.71 mL). The resulting mixture was stirred at rt for 16 h. The solvent was evaporated in vacio and the resulting residue was taken up in EtAOc and NaHCO₃(aq. sat. soltn). The organic layer was separated and discarded. The aqueous phase was purified by ion exchange chromatography (isolute SCX2 cartridge eluting with water and 7N solution of NH₃ in MeOH). The desired fractions were concentrated in vacuo to yield a solid that was taken up in MeOH/DCM. The solid was filtered off and the filtrate was evaporated in vacuo to yield intermediate 65 as yellow oil (40 mg, 49% yield).

### Preparation of Intermediate 66

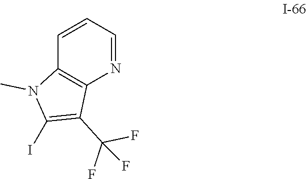

A solution of intermediate 34 (48.8 mg, 0.2 mmol), trifluoromethanesulfinic acid sodium salt (CAS: 2926-29-6; 94 mg, 0.6 mmol), ammonium persulfate (45.6 mg, 0.2 mmol) and [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (CAS: 870987-63-6; 2.25 mg, 0.002 mmol) in DMSO (2.74 mL) was prepared in a thin tube. The mixture was reacted in a vapourtec photoreactor with a residence time of 30 min at 40° C. in a 10 mL coil while irradiating with a 450 nm LED. The reaction mixture was then diluted with Et₂O and NaHCO₃(aq. sat. soltn, 25 mL). The organic phase was separated and kept. The aqueous layer was extracted with EtOAc (3×10 mL). The combined organic extracts were evaporated in vacuo. The residue thus obtained was added portionwise to a suspension of sodium hydride (7.8 mg, 0.195 mmol, 60% dispersion in mineral oil) in DMF (1 mL) at 0° C. The mixture was stirred at 0° C. for 10 min. Then methyl iodide (10.52 μL, 0.17 mmol) was added and the mixture was allowed to warm to rt and further stirred at rt for 1 h. NH₄Cl (aq. sat. soltn.) and EtOAc were added. The organic layer was separated, dried over Na₂SO₄, filtered and concentrated in vacuo. The residue thus obtained was purified by flash column chromatography (silica; EtOAc in DCM 0/100 to 100/0) and the desired fractions were concentrated in vacuo to yield intermediate 66 as an orange solid (24 mg, 57% yield).

### Preparation of Intermediate 67

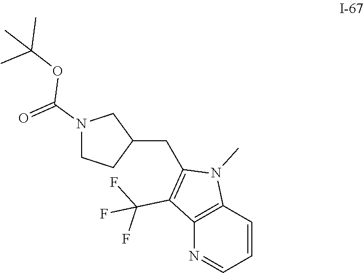 [SMILES: Cn1c(CC2CCN(C(=O)OC(C)(C)C)C2)c(C(F)(F)F)c2ncccc21]

Intermediate 37 (1.47 mmol, 0.24 M in THF) was added to a mixture of intermediate 66 (24 mg, 0.074 mmol), Pd(OAc)₂ (0.83 mg, 0.0037 mmol) and 2-dicyclohexylphosphino-2′,6′-diisopropoxy-1,1′-biphenyl (also known as RuPhos) (CAS: 787618-22-8; 3.43 mg, 0.0074 mmol). The resulting mixture was stirred at rt for 1 h. 10% aqueous NH₄Cl was added and the mixture was extracted with EtOAc. The organic layer was separated, dried over Na₂SO₄, filtered and concentrated in vacuo. The residue thus obtained was purified by flash column chromatography (silica; EtOAc in DCM, 0/100 to 100/0) and the desired fractions were concentrated in vacuo to yield intermediate 67 as yellow oil (17 mg, 60% yield).

### Preparation of Intermediate 68

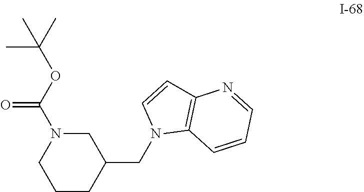 [SMILES: CC(C)(C)OC(=O)N1CCCC(Cn2ccc3ncccc32)C1]

Sodium hydride (19.1 mg, 0.48 mmol, 60% dispersion in mineral oil) was added portionwise to a solution of 4-azaindole (CAS: 272-49-1; 52 mg, 0.44 mmol) in DMF (4.12 mL) at rt. The resulting mixture was stirred at rt for 30 min. Then tert-butyl 3-(((methylsulfonyl)oxy)methyl)piperidine-1-carboxylate (CAS: 162166-99-6; 0.1 g, 0.34 mmol) was added and the mixture was further stirred at rt overnight. The mixture was poured in to ice and EtOAc was added. The organic layer was separated, washed with brine, dried over MgSO₄, filtered and concentrated in vacuo. The residue thus obtained was purified by flash column chromatography (silica; MeOH in DCM, 0/100 to 10/90) and the desired fractions were concentrated in vacuo to yield intermediate 68 as colorless oil (63 mg, 58% yield).

### Preparation of Intermediate 69

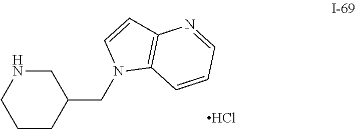 [SMILES: c1cnc2ccn(CC3CCCNC3)c2c1 ; Cl]

Hydrochloric acid (0.5 mL, 2 mmol, 4 M solution in 1,4-dioxane) was added to a solution of intermediate 68 (63 mg, 0.2 mmol) in 1,4-dioxane (0.5 mL) and the mixture was stirred at rt for 1 h. The volatiles were evaporated in vacuo to yield the HCl salt of intermediate 69 as a cream solid (50 mg, quantitative yield).

### B. Preparation of the Final Compounds

### Preparation of Product 1

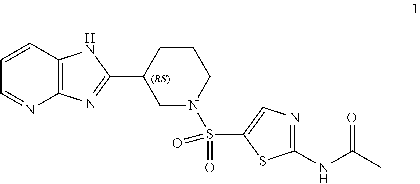 [SMILES: CC(=O)Nc1ncc(S(=O)(=O)N2CCCC(c3nc4ncccc4[nH]3)C2)s1]

2-Acetylamino-thiazole-5-sulfonyl chloride (CAS: 654072-71-6, 119 mg, 0.49 mmol) was added portion wise to a stirred solution of intermediate 1 (100 mg, 0.49 mmol) and diisopropylethylamine (0.17 mL, 0.99 mmol) in DCM (1.25 mL) at 0° C. and the mixture was further stirred at 0° C. for 1 h. The formed precipitate was filtered and dried in the vacuum oven affording product 1 as a white solid (80 mg, 40% yield).

### Preparation of Products 2, 3 and 4

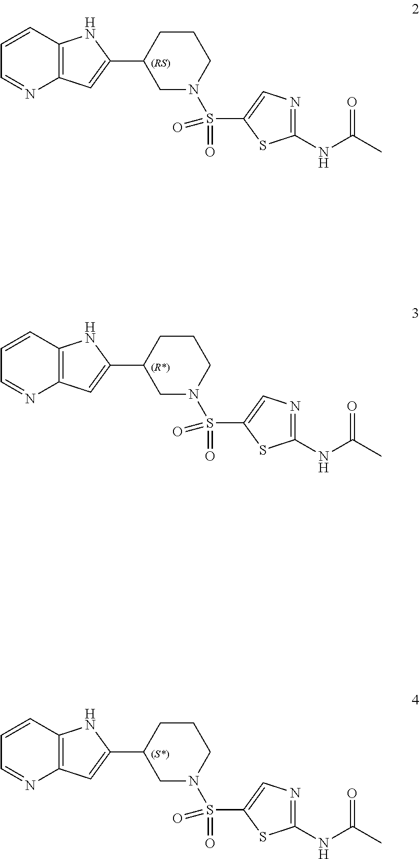 [SMILES: CC(=O)Nc1ncc(S(=O)(=O)N2CCCC(c3cc4ncccc4[nH]3)C2)s1 ; CC(=O)Nc1ncc(S(=O)(=O)N2CCCC(c3cc4ncccc4[nH]3)C2)s1 ; CC(=O)Nc1ncc(S(=O)(=O)N2CCCC(c3cc4ncccc4[nH]3)C2)s1]

N,N-diisopropylethylamine (0.69 mL, 4.15 mmol) and 2-acetylamino-thiazole-5-sulfonyl chloride (220 mg, 0.91 mmol) were added to a stirred solution of intermediate 5 (167 mg, 0.83 mmol) in THF (15 mL) at 0° C. under N₂ atmosphere. The mixture was allowed to warm to rt and then it was further stirred for 4 h. The mixture was diluted with NaHCO₃ (aq. sat. soltn.) and with DCM. The solid was filtered off and washed with water, MeOH and Et₂O and then dried under vacuum affording product 2 as a pale yellow solid (225 mg, 66% yield).

Product 2 (111 mg, 0.27 mmol) was then separated into enantiomers via chiral SFC [Stationary phase: Chiralpak IA 5 μm 250×20 mm, Mobile phase: 60% CO₂, 40% EtOH(0.3% iPrNH₂)] yielding product 3 (53 mg) and product 4 (49 mg).

### Preparation of Products 5, 6 and 7

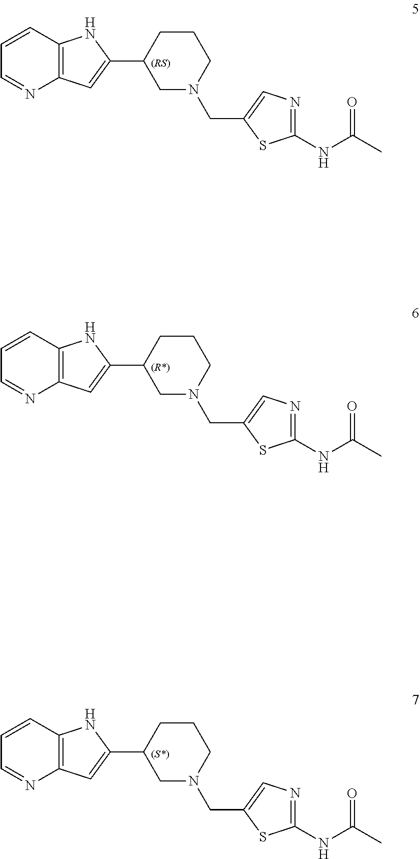 [SMILES: CC(=O)Nc1ncc(CN2CCCC(c3cc4ncccc4[nH]3)C2)s1 ; CC(=O)Nc1ncc(CN2CCCC(c3cc4ncccc4[nH]3)C2)s1 ; CC(=O)Nc1ncc(CN2CCCC(c3cc4ncccc4[nH]3)C2)s1]

Sodium triacetoxyborohydride (222 mg, 1 mmol) was added to a stirred solution of intermediate 5 (167 mg, 0.83 mmol), intermediate 40 (188 mg, 1 mmol) and triethylamine (0.231 mL, 1.66 mmol) in DCM (5 mL) at rt under N₂ atmosphere. The mixture was further stirred at rt for 1 h. The reaction mixture was diluted with NaHCO₃(aq. sat. soltn.) and DCM. The organic layer was separated, dried over MgSO₄, filtered and the filtrate was evaporated in vacuo. The residue thus obtained was purified by flash column chromatography (silica; DCM-MeOH (10:1,v/v) in DCM 0/100 to 50/50) and the desired fractions were concentrated in vacuo to yield product 5 as a white solid (116 mg, 39% yield).

Product 5 (106 mg, 0.3 mmol) was separated into enantiomers via chiral SFC [Stationary phase: CHIRALPAK IC 5 μm 250×20 mm, Mobile phase: 50% CO₂, 50% EtOH (0.3% iPrNH₂)] yielding product 6 (45 mg) and product 7 (45 mg).

### Preparation of Product 5

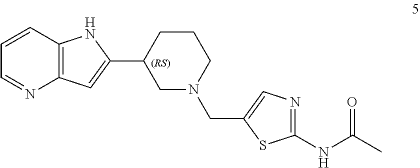 [SMILES: CC(=O)Nc1ncc(CN2CCCC(c3cc4ncccc4[nH]3)C2)s1]

Intermediate 42 (314 mg, 1.65 mmol) was added to a stirred suspension of intermediate 5 (332 mg, 1.65 mmol) and triethylamine (1.38 mL, 9.9 mmol) in DCM (10.2 mL) and DMF (1.3 mL) at 0° C. The mixture was allowed to warm to rt and further stirred at rt for 1 h. Then the solvent was evaporated in vacuo and the residue thus obtained was purified by RP HPLC (Stationary phase: C18 XBridge 50×150 mm 5 um), Mobile phase: Gradient from 80% 10 mM NH₄CO₃H pH 9 solution in Water, 20% MeOH to 0% 10 mM NH₄CO₃H pH 9 solution in Water, 100% MeOH), yielding product 5 (341 mg; 58% yield) as a white solid.

### Preparation of Product 8

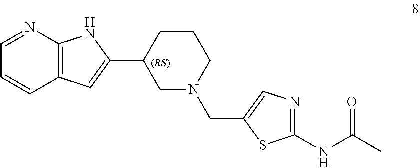 [SMILES: CC(=O)Nc1ncc(CN2CCCC(c3cc4cccnc4[nH]3)C2)s1]

Sodium triacetoxyborohydride (55.4 mg, 0.26 mmol) was added to a solution of 2-piperidin-3-yl-1H-pyrrolo[2,3-b]pyridine dihydrochloride (CAS: 1185303-84-7; 51.8 mg, 0.21 mmol), intermediate 40 (44.5 mg, 0.26 mmol) and triethylamine (0.091 mL, 0.65 mmol) in DCM (1.3 mL), then the mixture was stirred at rt for 8 h. MeOH (1 mL) followed by sodium borohydride (24.7 mg, 0.65 mmol) were added and the mixture was stirred for 8 h. Then aqueous NaHCO₃(aq. sat. soltn.) was added and the organic layer was separated, dried (MgSO₄), filtered and concentrated in vacuo. The crude product was purified by flash column chromatography (SiO₂, EtOAc in heptane 0/100 to 80/20). The desired fractions were collected and the solvents evaporated in vacuo to yield product 8 (33 mg, 43% yield) as white solid.

### Preparation of Product 9

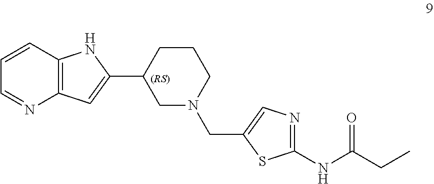 [SMILES: CCC(=O)Nc1ncc(CN2CCCC(c3cc4ncccc4[nH]3)C2)s1]

Sodium triacetoxyborohydride (90 mg, 0.42 mmol) was added to a stirred solution of intermediate 5 (130 mg, 0.302 mmol), intermediate 44 (67 mg, 0.36 mmol) and triethylamine (0.15 mL, 1.21 mmol) in DCM (5 mL) at rt. The mixture was further stirred at rt overnight. The reaction mixture was diluted with NaHCO₃(aq. sat. soltn.) and DCM. The organic layer was separated, dried over MgSO₄, filtered and the filtrate was evaporated in vacuo. The residue thus obtained was purified by reverse phase HPLC from 90% [65 mM NH₄OAc+ACN (90:10)]-10% [ACN: MeOH 1:1] to 54% [65 mM NH₄OAc+ACN (90:10)]-46% [ACN: MeOH 1:1]. The desired fractions were concentrated in vacuo to yield product 9 (113 mg, 42% yield).

### Preparation of Product 10

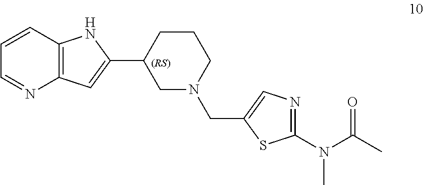 [SMILES: CC(=O)N(C)c1ncc(CN2CCCC(c3cc4ncccc4[nH]3)C2)s1]

Sodium triacetoxyborohydride (172.6 mg, 0.814 mmol) was added to a stirred solution of intermediate 5 (97 mg, 0.41 mmol, hydrochloric acid salt), intermediate 45 (90 mg, 0.49 mmol) and triethylamine (0.2 mL, 1.63 mmol) in DCM (12 mL) at rt. The mixture was further stirred at rt overnight. The reaction mixture was quenched with NaHCO₃(aq. sat. soltn.) and diluted with DCM. The organic layer was separated, dried over MgSO₄, filtered and the filtrate was evaporated in vacuo. The residue thus obtained was purified by reverse phase HPLC from 81% H₂O (25 mM NH₄HCO₃)-19% MeCN-MeOH to 45% H₂O (25 mM NH₄HCO₃)-55% MeCN-MeOH. The desired fractions were concentrated in vacuo to yield product 10 (15 mg, 10% yield).

### Preparation of Product 11

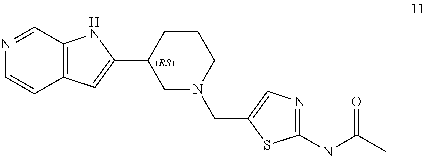 [SMILES: CC(=O)Nc1ncc(CN2CCCC(c3cc4ccncc4[nH]3)C2)s1]

To a solution of intermediate 11 (40 mg, 0.2 mmol), in DCM (1 mL) and MeOH (0.1 mL), intermediate 40 (37.2 mg, 0.22 mmol) was added and the reaction mixture was stirred at rt for 3 h. Then sodium cyanoborohydride (25 mg, 0.4 mmol) was added and the reaction mixture was stirred at rt for 24 h. Then NaHCO₃(aq. sat. soltn.) was added and the product extracted with DCM. The organic layer was separated, dried (MgSO₄), filtered and the solvents evaporated in vacuo. The product was purified by RP HPLC (Stationary phase: C₁₈ X Bridge 30×100 mm 5 um), Mobile phase: Gradient from 80%/0.1% NH₄CO₃H/NH₄OH pH 9 solution in water, 20% CH₃CN to 0%/0.1% NH₄CO₃H/NH₄OH pH 9 solution in water, 100% CH₃CN). The desired fractions were collected and concentrated in vacuo yielding a product that was further purified by flash column chromatography (silica; MeOH in DCM 0/100 to 10/90). The desired fractions were collected and concentrated in vacuo yielding product 11 (70.6 mg, 59% yield) as a white solid.

### Preparation of Products 12, 13 and 14

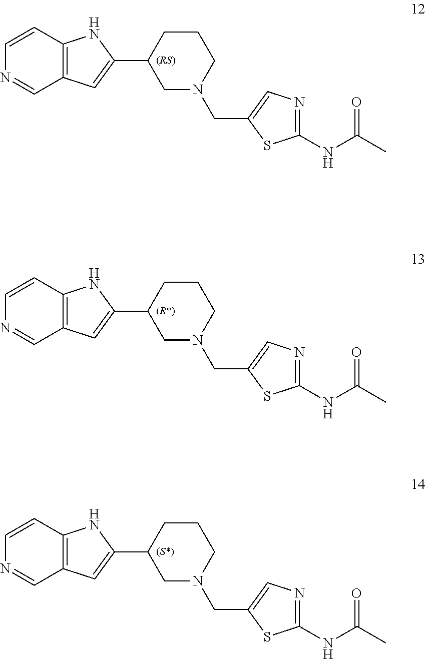 [SMILES: CC(=O)Nc1ncc(CN2CCCC(c3cc4cnccc4[nH]3)C2)s1 ; CC(=O)Nc1ncc(CN2CCCC(c3cc4cnccc4[nH]3)C2)s1 ; CC(=O)Nc1ncc(CN2CCCC(c3cc4cnccc4[nH]3)C2)s1]

To a solution of intermediate 8 (40 mg, 0.2 mmol), in DCM (1 mL) and MeOH (0.1 mL), intermediate 40 (37.2 mg, 0.22 mmol) was added and the reaction mixture was stirred at rt for 3 h. Then sodium cyanoborohydride (25 mg, 0.4 mmol) was added and the reaction mixture was stirred at rt for 24 h. Then NaHCO₃(aq. sat. soltn.) was added and the product extracted with DCM. The organic layer was separated, dried (MgSO₄), filtered and the solvents evaporated in vacuo. The product was purified by RP HPLC (Stationary phase: C₁₈ X Bridge 30×100 mm 5 um), Mobile phase: Gradient from 80%/0.1% NH₄CO₃H/NH₄OH pH 9 solution in water, 20% CH₃CN to 0%/0.1% NH₄CO₃H/NH₄OH pH 9 solution in water, 100% CH₃CN). The desired fractions were collected and concentrated in vacuo yielding a product that was further purified by flash column chromatography (silica; MeOH in DCM 0/100 to 10/90). The desired fractions were collected and concentrated in the vacuum oven at 50° C. yielding product 12 (32 mg, 45% yield) as a white solid.

Product 12 (198 mg, 0.56 mmol) was then separated into enantiomers via chiral SFC [Stationary phase: Chiralpak AD-H 5 μm 250×30 mm, Mobile phase: 55% CO₂, 45% EtOH (0.3% iPrNH₂)] yielding product 13 (76 mg) and product 14 (73 mg).

### Preparation of Products 15, 16 and 17

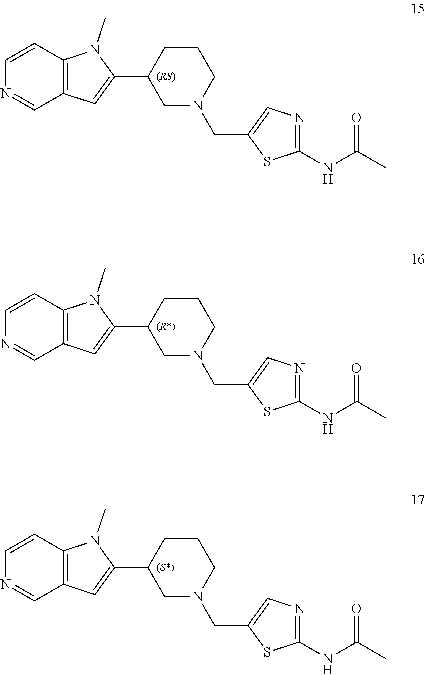 [SMILES: CC(=O)Nc1ncc(CN2CCCC(c3cc4cnccc4n3C)C2)s1 ; CC(=O)Nc1ncc(CN2CCCC(c3cc4cnccc4n3C)C2)s1 ; CC(=O)Nc1ncc(CN2CCCC(c3cc4cnccc4n3C)C2)s1]

Sodium cyanoborohydride (374 mg, 5.96 mmol) was added to a solution of intermediate 27 (500 mg, 1.99 mmol), intermediate 40 (406 mg, 2.38 mmol) and triethylamine (0.69 mL, 5 mmol) in DCM (10.6 mL) and MeOH (0.16 mL) and the reaction mixture was stirred at rt for 16 h. Then water was added and the product extracted with DCM. The organic layer was separated, dried (Na₂SO₄), filtered and the solvents evaporated in vacuo. The resulting residue was purified by flash column chromatography (silica; 7 M solution of ammonia in MeOH in EtOAc 0/100 to 5/95). The desired fractions were collected and concentrated in vacuo. The resultant solid was diluted in EtOH/water and stirred at 160° C. for 20 min under microwave irradiation. Then the mixture was purified by RP HPLC (Stationary phase: C₁₈ X Bridge 30×100 mm 5 um), Mobile phase: Gradient from 81% 10 mM NH₄CO₃H pH 9 solution in water, 19% CH₃CN to 64% 10 mM NH₄CO₃H pH 9 solution in water, 36% CH₃CN), yielding product 15 (230 mg, 31% yield) as yellow solid.

Product 15 (501 mg) was then separated into enantiomers via chiral SFC (Stationary phase: Chiralpak AS-H 5 μm 250×20 mm, mobile phase: 65% CO₂, 35% MeOH) yielding product 16 (190 mg) and product 17 (190 mg) as pale yellow solids.

### Preparation of Product 18

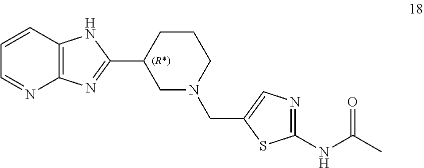 [SMILES: CC(=O)Nc1ncc(CN2CCCC(c3nc4ncccc4[nH]3)C2)s1]

Intermediate 42 (51 mg, 0.252 mmol) was added to a stirred solution of intermediate 1a (57.3 mg, 0.252 mmol) and triethylamine (0.175 mL, 1.26 mmol) in ACN (0.82 mL) at 0° C. The mixture was stirred at 0° C. for 1 h, then at rt for 72 h. The mixture was diluted with water and extracted with EtOAc. The organic layer was separated, dried (Na₂SO₄), filtered and concentrated in vacuo. The resultant solid was purified by flash column chromatography (silica, 7M solution of ammonia in MeOH in EtOAc from 0/100 to 10/90). The desired fractions were collected and concentrated in vacuo to yield product 18 (10 mg, 11% yield) as white solid.

### Preparation of Product 19

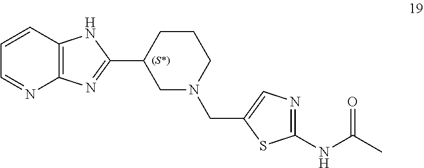 [SMILES: CC(=O)Nc1ncc(CN2CCCC(c3nc4ncccc4[nH]3)C2)s1]

Intermediate 42 (51 mg, 0.252 mmol) was added to a stirred solution of intermediate 1b (57.3 mg, 0.252 mmol) and triethylamine (0.175 mL, 1.26 mmol) in ACN (0.82 mL) at 0° C. The mixture was stirred at 0° C. for 1 h, then at rt for 72 h. The mixture was diluted with water and extracted with EtOAc. The organic layer was separated, dried (Na₂SO₄), filtered and concentrated in vacuo. The resultant solid was purified by flash column chromatography (silica, 7M solution of ammonia in MeOH in EtOAc from 0/100 to 10/90). The desired fractions were collected and concentrated in vacuo to yield product 19 (15 mg, 17% yield) as white solid.

### Preparation of Product 20

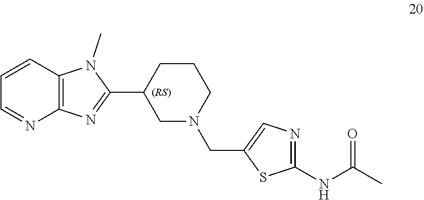 [SMILES: CC(=O)Nc1ncc(CN2CCCC(c3nc4ncccc4n3C)C2)s1]

Intermediate 43 (30 mg, 0.14 mmol) was added to a stirred solution of intermediate 31 (35.6 mg, 0.14 mmol) and triethylamine (0.096 mL, 0.69 mmol) in ACN (0.45 mL) at 0° C. The mixture was stirred at 0° C. for 1 h, then at rt for 72 h. The mixture was diluted with water and extracted with EtOAc. The organic layer was separated, dried (Na₂SO₄), filtered and concentrated in vacuo. The resultant solid was triturated in ACN, filtered and dried in vacuo to yield product 20 (28 mg, 54% yield) as white solid.

### Preparation of Product 21

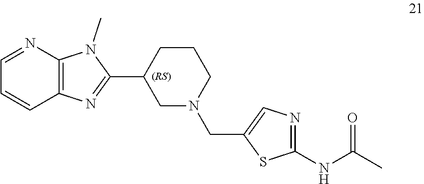 [SMILES: CC(=O)Nc1ncc(CN2CCCC(c3nc4cccnc4n3C)C2)s1]

Intermediate 42 (250 mg, 1.31 mmol) was added to a stirred solution of intermediate 28 (283.6 mg, 1.31 mmol) and triethylamine (0.911 mL, 6.56 mmol) in ACN (4.3 mL) at 0° C. The mixture was stirred at 0° C. for 1 h, then at rt for 72 h. The mixture was diluted with water and extracted with EtOAc. The organic layer was separated, dried (Na₂SO₄), filtered and concentrated in vacuo. The resultant solid was purified by RP HPLC

(Stationary phase: C₁₈ X Bridge 30×100 mm 5 um), Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo to yield product 21 (25 mg, 5% yield) as white solid.

### Preparation of Products 22 and 23

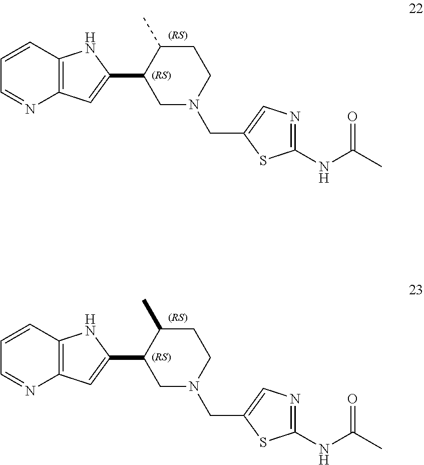

Sodium triacetoxyborohydride (160.6 mg, 0.76 mmol) was added to a solution of intermediate 16 (159 mg, 0.63 mmol, hydrochloric acid salt), intermediate 40 (129 mg, 0.76 mmol) and triethylamine (0.263 mL, 1.89 mmol) in DCM (3.8 mL), then the mixture was stirred at rt for 8 h. MeOH (2.89 mL) followed by sodium borohydride (71 mg, 1.89 mmol) were added and the mixture was stirred for 72 h. Then aqueous NaHCO₃(aq. sat. soltn.) was added and the organic layer was separated, dried (MgSO₄), filtered and concentrated in vacuo. The crude product was purified by flash column chromatography (SiO₂, EtOAc in heptane 0/100 to 80/20). The desired fractions were collected and the solvents evaporated in vacuo to yield a mixture containing product 22 and product 23: This mixture was purified by RP HPLC (Stationary phase: C₁₈ X Bridge 30×100 mm 5 μm), Mobile phase: Gradient from 74%/0.1% NH₄CO₃H/NH₄OH pH 9 solution in water, 26% CH₃CN to 58%/0.1% NH₄CO₃H/NH₄OH pH 9 solution in water, 42% CH₃CN) yielding product 22 (35 mg, 15% yield) as yellow solid and product 23 (8 mg, 3.42% yield) as yellow film.

### Preparation of Product 24

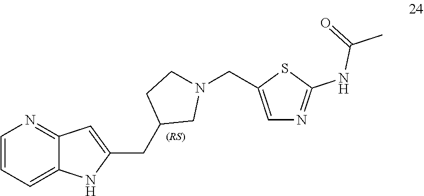 [SMILES: CC(=O)Nc1ncc(CN2CCC(Cc3cc4ncccc4[nH]3)C2)s1]

Trifluoroacetic acid (1 mL, 13.07 mmol) was added to a solution of intermediate 38 (55 mg, 0.17 mmol) in DCM (1 mL). The solution was stirred for 30 min at rt. Then the solvent was evaporated. The residue was taken up in DCM (1 mL) and acetic acid (0.1 mL) was added. Then intermediate 40 (59 mg, 0.34 mmol) and sodium triacetoxy-borohydride (147.3 mg, 0.695 mmol) were added. The mixture was stirred at rt overnight. Then NaHCO₃(aq. sat. soltn.) was added and the mixture was extracted with DCM/MeOH 4/1 (5×). The combined organic extracts were dried (Na₂SO₄), filtered and the solvent evaporated. The residue was purified by column chromatography (silica, MeOH in EtOAc 0/100 to 100/0). Desired fractions were collected, the solvent evaporated and the residue thus obtained was further purified by RP HPLC (Stationary phase: C₁₈ X Bridge 30×100 mm 5 μm), Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo to yield product 24 (6 mg, 7% yield) as an off-white solid.

### Preparation of Product 25

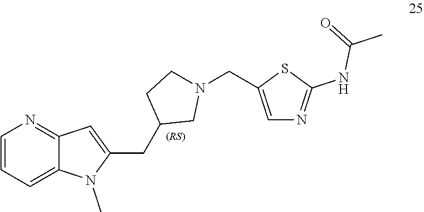 [SMILES: CC(=O)Nc1ncc(CN2CCC(Cc3cc4ncccc4n3C)C2)s1]

Trifluoroacetic acid (1 mL, 13.07 mmol) was added to a solution of intermediate 39 (63 mg, 0.2 mmol) in DCM (1 mL). The solution was stirred for 30 min at rt. Then the solvent was evaporated. The residue was taken up in DCM (1 mL) and acetic acid (0.12 mL) was added. Then intermediate 40 (51 mg, 0.3 mmol) and sodium triacetoxy borohydride (106 mg, 0.49 mmol) were added. The mixture was stirred at rt for 2 h and then additional sodium triacetoxy borohydride (106 mg, 0.49 mmol) was added. The mixture was stirred at rt overnight. Then NaHCO₃(aq. sat. soltn.) was added and the mixture was extracted with DCM. The combined organic extracts were dried (Na₂SO₄), filtered and the solvent evaporated. The residue was purified by column chromatography (silica, MeOH in EtOAc 0/100 to 50/50). Desired fractions were collected, the solvent evaporated and the resulting solid was triturated with Et₂O, filtered and dried in the vacuum oven (50° C.) to yield product 25 (22 mg, 30% yield) as an off-white solid.

### Preparation of Product 26

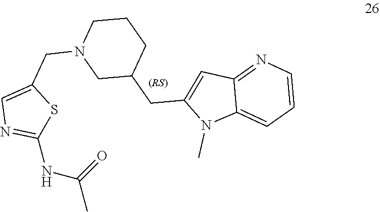 [SMILES: CC(=O)Nc1ncc(CN2CCCC(Cc3cc4ncccc4n3C)C2)s1]

Trifluoroacetic acid (1 mL, 13.07 mmol) was added to a solution of intermediate 36 (55 mg, 0.17 mmol) in DCM (1 mL). The solution was stirred for 30 min at rt. Then the solvent was evaporated. The residue was taken up in DCM (1 mL) and acetic acid (0.1 mL) was added. Then intermediate 40 (42.6 mg, 0.25 mmol) and sodium triacetoxy-borohydride (88.5 mg, 0.41 mmol) were added. The mixture was stirred at rt for 3 h and then additional sodium triacetoxy borohydride (88.5 mg, 0.41 mmol) was added. The mixture was stirred at rt overnight. Then NaHCO₃(aq. sat. soltn.) was added and the mixture was extracted with DCM. The combined organic extracts were dried (Na₂SO₄), filtered and the solvent evaporated. The residue was purified by column chromatography (silica, MeOH in EtOAc 0/100 to 50/50). Desired fractions were collected, the solvent evaporated and the resulting residue was treated with diisopropyl ether and the resulting solid was filtered and dried in the vacuum oven (50° C.) to yield product 26 (25 mg, 64% yield) as an off-white solid.

### Preparation of Product 27

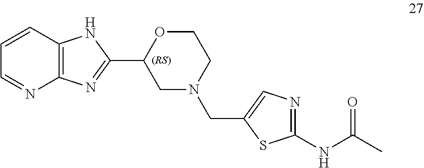 [SMILES: CC(=O)Nc1ncc(CN2CCOC(c3nc4ncccc4[nH]3)C2)s1]

Sodium triacetoxyborohydride (127 mg, 0.6 mmol) was added to a stirred solution of intermediate (120 mg, 0.5 mmol, hydrochloric acid salt), intermediate 40 (102 mg, 0.6 mmol) and triethylamine (0.21 mL, 1.5 mmol) in DCM (3 mL) at rt. The mixture was further stirred at rt for 56 h. The reaction mixture was quenched with NaHCO₃(aq. sat. soltn.) and the organic layer was separated, dried over MgSO₄, filtered and the filtrate was evaporated in vacuo. The residue thus obtained was purified by reverse phase HPLC (Stationary phase: C₁₈ X Bridge 30×100 mm 5 μm), Mobile phase: Gradient from 80%/0.1% NH₄CO₃H/NH₄OH pH 9 solution in water, 20% CH₃CN to 0%/0.1% NH₄CO₃H/NH₄OH pH 9 solution in water, 100% CH₃CN). The desired fractions were concentrated in vacuo to yield product 27 (18 mg, 10% yield) as a pale beige solid.

### Preparation of Products 28, 29, 30, 31, 32

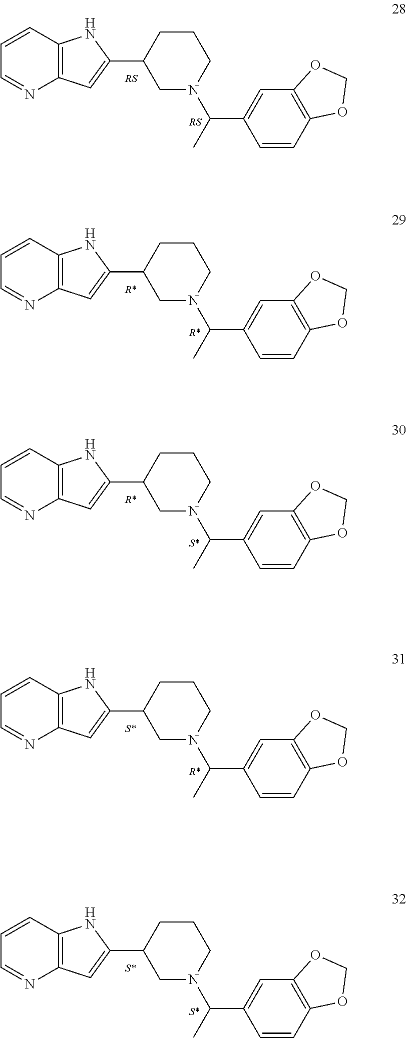 [SMILES: CC(c1ccc2c(c1)OCO2)N1CCCC(c2cc3ncccc3[nH]2)C1 ; CC(c1ccc2c(c1)OCO2)N1CCCC(c2cc3ncccc3[nH]2)C1 ; CC(c1ccc2c(c1)OCO2)N1CCCC(c2cc3ncccc3[nH]2)C1 ; CC(c1ccc2c(c1)OCO2)N1CCCC(c2cc3ncccc3[nH]2)C1 ; CC(c1ccc2c(c1)OCO2)N1CCCC(c2cc3ncccc3[nH]2)C1]

A solution of intermediate 51 (400 mg, 1.14 mmol) in NMP (3 mL) was added to potassium tert-butoxide (270 mg, 2.4 mmol) in NMP (3 mL) and the mixture was stirred at rt overnight. NaHCO₃(aq. sat. soltn.) and EtOAc were added. The organic layer was separated, washed with water and brine, dried over Na₂SO₄, filtered and concentrated in vacuo. The resultant residue was purified by flash chromatography (silica; MeOH in DCM, 0/100 to 100/0). The desired fractions were concentrated in vacuo to yield product 28 as a sticky solid (250 mg, 62% yield).

Product 28 (220 mg, 0.63 mmol) was then separated into enantiomers via chiral SFC [Stationary phase: Chiralpak AS-H 5 μm 250*20 mm, mobile phase: 80% CO₂, 20% mixture of Et₀H/iPrOH 50/50 v/v (+0.3% iPrNH₂)] yielding product 29 (36 mg), product 30 (37 mg), product 31 (44 mg), and product 32 (45 mg).

### Preparation of Products 33, 34, 35, 36, 37

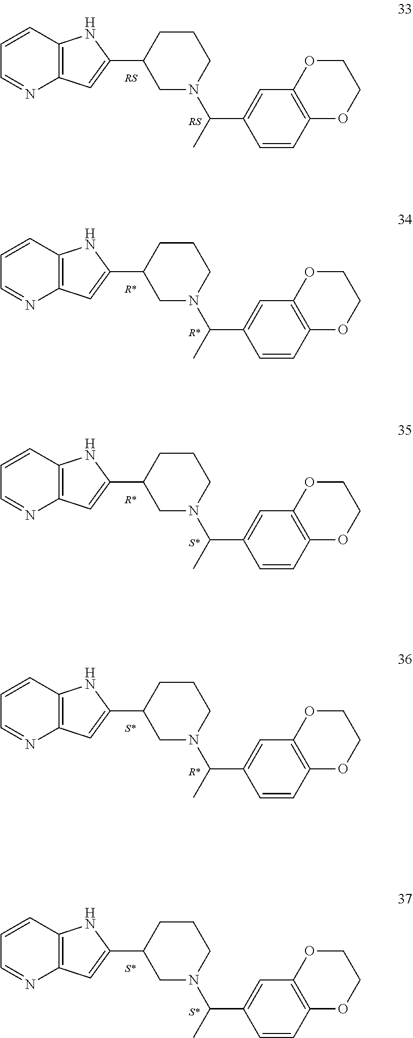 [SMILES: CC(c1ccc2c(c1)OCCO2)N1CCCC(c2cc3ncccc3[nH]2)C1 ; CC(c1ccc2c(c1)OCCO2)N1CCCC(c2cc3ncccc3[nH]2)C1 ; CC(c1ccc2c(c1)OCCO2)N1CCCC(c2cc3ncccc3[nH]2)C1 ; CC(c1ccc2c(c1)OCCO2)N1CCCC(c2cc3ncccc3[nH]2)C1 ; CC(c1ccc2c(c1)OCCO2)N1CCCC(c2cc3ncccc3[nH]2)C1]

A solution of intermediate 56 (250 mg, 0.69 mmol) in NMP (3 mL) was added to potassium tert-butoxide (162 mg, 1.44 mmol) in NMP (3 mL) and the mixture was stirred at rt overnight. NH₄Cl (2 mL, aq. sat. soltn.), water (10 mL) and EtOAc were added. The organic layer was separated, dried over MgSO₄, filtered and concentrated in vacuo. The resultant residue was purified by flash chromatography (silica; MeOH in DCM, 0/100 to 80/20). The desired fractions were concentrated in vacuo to yield product 33 (105 mg, 42% yield).

Product 33 (104 mg) was then separated into enantiomers via chiral SFC [Stationary phase: Chiralpak AS-H 5 μm 250*20 mm, mobile phase: 80% CO₂, 20% iPrOH (0.3% iPrNH₂)] yielding impure product 34 (31 mg), impure product 35 (20 mg), impure product 36 (21 mg) and impure product 37 (23 mg).

Impure product 34 (31 mg) was purified via preparative LC (Stationary phase: irregular bare silica 10 g, mobile phase: 0.5% NH₄OH, 94% DCM, 6% MeOH) yielding pure product 34 (18 mg) as yellow oil.

Impure product 35 (20 mg) was purified via preparative LC (Stationary phase: irregular bare silica 10 g, mobile phase: 0.5% NH₄OH, 94% DCM, 6% MeOH) yielding pure product 35 (19 mg) as yellow oil.

Impure product 36 (21 mg) was purified via preparative LC (Stationary phase: irregular bare silica 10 g, mobile phase: 0.5% NH₄OH, 94% DCM, 6% MeOH) yielding pure product 36 (17 mg) as yellow oil.

Impure product 37 (23 mg) was purified via preparative LC (Stationary phase: irregular bare silica 10 g, mobile phase: 0.5% NH₄OH, 94% DCM, 6% MeOH) yielding pure product 37 (15 mg) as yellow oil.

### Preparation of Product 33

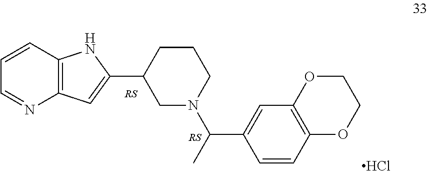 [SMILES: CC(c1ccc2c(c1)OCCO2)N1CCCC(c2cc3ncccc3[nH]2)C1 ; Cl]

A mixture of Intermediate 5 (210 mg, 1.04 mmol) and 6-acetyl-1,4-benzodioxane (CAS: 2879-20-1; 223 mg, 1.25 mmol) in Ti(OⁱPr)₄ (3.1 mL, 10.4 mmol) were stirred at 75° C. under N₂ atmosphere for 4 h. The reaction mixture was cooled to 0° C. under N₂ atmosphere and 1,2-dichloroethane (1 mL), MeOH (15 mL) and sodium borohydride (118 mg, 3.13 mmol) were sequentially added. The reaction mixture was allowed to warm to rt and further stirred at rt for 3 h. Water and DCM were added. The mixture was filtered through a Celite® pad and the volatiles were evaporated in vacuo. The residue thus obtained was taken up in DCM and HCl (1 mL, 4N in 1,4-dioxane) was added. The volatiles were evaporated in vacuo and the resulting residue was treated with Et₂O to give a solid that was filtered and dried to yield product 33 as hydrochloric acid salt (29 mg, 7% yield).

### Preparation of Product 38

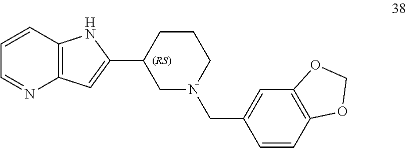 [SMILES: c1cnc2cc(C3CCCN(Cc4ccc5c(c4)OCO5)C3)[nH]c2c1]

Sodium triacetoxyborohydride (92 mg, 0.43 mmol) was added to a stirred solution of intermediate 5 (130 mg, 0.302 mmol, bis trifluoroacetate salt), piperonal (56 mg, 0.37 mmol) and triethylamine (0.15 mL, 1.23 mmol) in DCM (5 mL) at rt. The mixture was further stirred at rt overnight. The reaction mixture was diluted with NaHCO₃(aq. sat. soltn.) and DCM. The organic layer was separated, dried over MgSO₄, filtered and the filtrate was evaporated in vacuo. The residue thus obtained was purified by reverse phase HPLC from 70% [25 mM NH₄HCO₃]-30% [ACN: MeOH 1:1] to 27% [25 mM NH₄HCO₃]-73% [ACN: MeOH 1:1]. The desired fractions were concentrated in vacuo to yield product 38 (33 mg, 31% yield) as a pale solid.

### Preparation of Product 39

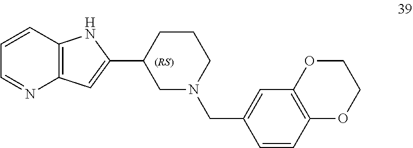 [SMILES: c1cnc2cc(C3CCCN(Cc4ccc5c(c4)OCCO5)C3)[nH]c2c1]

Sodium triacetoxyborohydride (150 mg, 0.71 mmol) was added to a stirred solution of intermediate 5 (120 mg, 0.50 mmol, hydrochloric acid salt), 1,4-benzodioxan-6-carboxaldehyde (CAS: 29668-44-8; 83 mg, 0.50 mmol) and triethylamine (0.14 mL, 1.01 mmol) in DCM (5 mL) at rt. The mixture was further stirred at rt overnight. The reaction mixture was diluted with NaHCO₃(aq. sat. soltn.) and DCM. The organic layer was separated, dried over MgSO₄, filtered and the filtrate was evaporated in vacuo. The resultant residue was purified by flash chromatography (silica; EtOAc in heptane, 0/100 to 60/40). The desired fractions were concentrated in vacuo. The residue thus obtained was taken up in DCM and HCl (0.039 mL, 4N in 1,4-dioxane) was added. The volatiles were evaporated in vacuo and the resulting residue was treated with diisopropylether to give a solid that was filtered and dried to yield to yield product 39 (53 mg, 27% yield, hydrochloric acid salt) as a pale solid.

### Preparation of Product 40

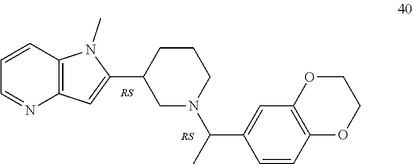 [SMILES: CC(c1ccc2c(c1)OCCO2)N1CCCC(c2cc3ncccc3n2C)C1]

Sodium cyanoborohydride (18.7 mg, 0.3 mmol) was added to a mixture of intermediate 27 (50 mg, 0.2 mmol, hydrochloric acid salt), 6-acetyl-1,4-benzodioxane (CAS: 2879-20-1; 70.7 mg, 3.97 mmol), triethylamine (0.069 mL, 0.5 mmol) and Ti(OⁱPr)₄ (0.076 mL, 0.26 mmol) in anhydrous MeOH (0.48 mL). The resulting suspension was stirred at rt for 16 h and then the mixture was heated at 70° C. and further stirred for 16 h. The mixture was cooled to rt and then filtered through a Celite® pad and the volatiles were evaporated in vacuo. Water and EtOAc were added. The organic phase was separated, dried over Na₂SO₄, filtered and the filtrate was evaporated in vacuo. The resultant oil was purified by RP HPLC (Stationary phase: C₁₈ X Bridge 30×100 mm 5 μm), mobile phase: gradient from 47% 10 mM NH₄CO₃H pH 9 solution in water, 53% MeOH to 24% 10 mM NH₄CO₃H pH 9 solution in water, 76% MeOH) to yield product 40 as a white solid (20 mg, 27% yield).

### Preparation of Products 41, 42 and 43

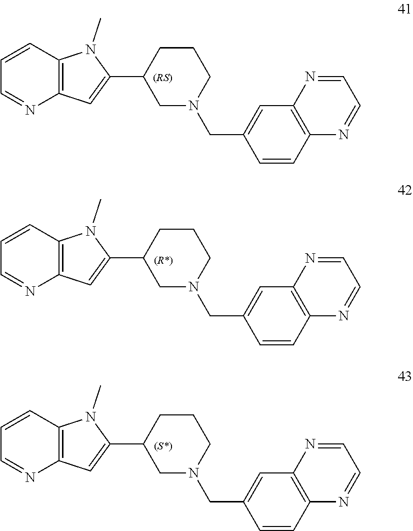 [SMILES: Cn1c(C2CCCN(Cc3ccc4nccnc4c3)C2)cc2ncccc21 ; Cn1c(C2CCCN(Cc3ccc4nccnc4c3)C2)cc2ncccc21 ; Cn1c(C2CCCN(Cc3ccc4nccnc4c3)C2)cc2ncccc21]

Triethylamine (0.29 mL, 2.01 mmol) was added to a stirred solution of intermediate 27 (150 mg, 0.52 mmol, bis hydrochloric acid salt) in DCM (2.5 mL) and the mixture was stirred at rt for 2 min. Then 6-quinoxalinecarboxaldehyde (CAS: 130345-50-5; 82.3 mg, 0.52 mmol) followed by sodium cyanoborohydride (46 mg, 0.73 mmol) were added at rt. The mixture was further stirred at rt for 15 h. The reaction mixture was diluted with NaHCO₃(aq. sat. soltn.) and DCM. The organic layer was separated, dried over MgSO₄, filtered and the filtrate was evaporated in vacuo. The residue was purified by RP HPLC (Stationary phase: C₁₈ X Bridge 30×100 mm 5 μm), Mobile phase: Gradient from 81% 10 mM NH₄CO₃H pH 9 solution in water, 19% CH₃CN to 64% 10 mM NH₄CO₃H pH 9 solution in water, 36% CH₃CN), to yield product 41 (82 mg, 44%) as a white solid.

Product 41 (111 mg) was separated into enantiomers via chiral SFC [Stationary phase: CHIRALCEL OJ-H 5 μm 250×20 mm, Mobile phase: 83% CO₂, 17% MeOH (0.3% iPrNH₂)] yielding product 42 (46 mg) and product 43 (47 mg).

### Preparation of Products 44, 45, 46, 47, 48

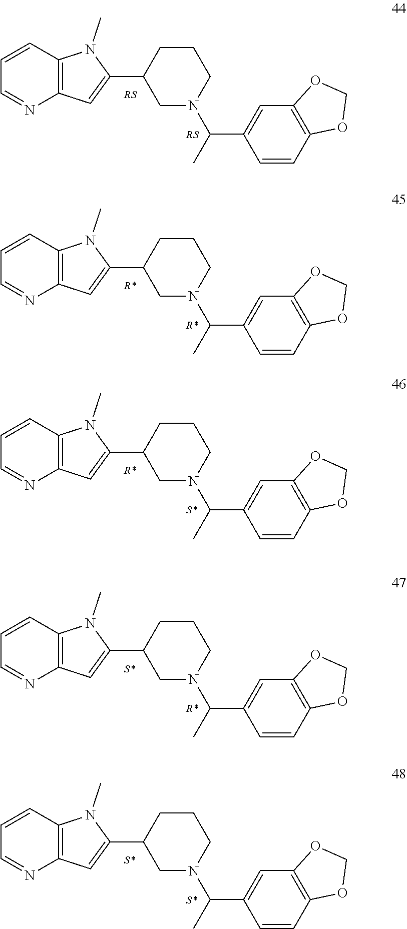 [SMILES: CC(c1ccc2c(c1)OCO2)N1CCCC(c2cc3ncccc3n2C)C1 ; CC(c1ccc2c(c1)OCO2)N1CCCC(c2cc3ncccc3n2C)C1 ; CC(c1ccc2c(c1)OCO2)N1CCCC(c2cc3ncccc3n2C)C1 ; CC(c1ccc2c(c1)OCO2)N1CCCC(c2cc3ncccc3n2C)C1 ; CC(c1ccc2c(c1)OCO2)N1CCCC(c2cc3ncccc3n2C)C1]

Piperonal (346 mg, 2.3 mmol) and Ti(O'Pr)₄ (0.68 mL, 2.3 mmol) were added to a solution of intermediate 27 (331 mg, 1.54 mmol) in anhydrous DCM (4.73 mL) and the resulting mixture was stirred at rt for 18 h. Then the reaction was cooled to 0° C. and methylmagnesium bromide (5.49 mL, 1.4 M solution in THF) was added dropwise followed by anhydrous THF (4.73 mL). The mixture was further stirred at 0° C. for 5 min and then at rt for 3 h. The reaction mixture was diluted with NH₄Cl (aq. sat. soltn.) and DCM. The organic layer was separated, dried over MgSO₄, filtered and the filtrate was evaporated in vacuo. The resultant residue was purified by flash chromatography (silica; EtOAc in heptane, 5/100 to 30/70). The desired fractions were concentrated in vacuo to yield product 44 (65 mg, 11.5% yield).

Product 44 (133 mg) was separated into enantiomers via chiral SFC [stationary phase: Chiralpak AD-H 5 μm 250*30 mm, Mobile phase: 70% CO₂, 30% MeOH (0.3% iPrNH₂)] impure product 45 (32 mg), impure product 46 (26 mg), pure product 47 (32 mg) and impure product 48 (26 mg).

Impure product 45 (32 mg) was purified via preparative LC (Stationary phase: irregular bare silica 40 g, mobile phase: 60% heptane, 5% MeOH (+5% NH₄OH), 35% EtOAc) yielding pure product 45 (24 mg) as pale yellow oil.

Impure product 46 (26 mg) was purified via preparative LC (Stationary phase: irregular bare silica 24 g, mobile phase: 60% heptane, 5% MeOH (+5% NH₄OH), 35% EtOAc) yielding impure product 46 (17 mg) as pale yellow oil. Impure product 46 (17 mg) was purified via reverse phase HPLC (Stationary phase: C₁₈ X Bridge 30×100 mm 5 μm), mobile phase: gradient from 75% 10 mM NH₄CO₃H pH 9 solution in water, 25% CH₃CN to 57% 10 mM NH₄CO₃H pH 9 solution in Water, 43% CH₃CN), yielding pure product 46 (6.1 mg) as colorless oil.

Impure product 48 (26 mg) was purified via preparative LC (Stationary phase: irregular bare silica 40 g, mobile phase: 60% heptane, 5% MeOH (+5% NH₄OH), 35% EtOAc) yielding pure product 48 (23 mg) as pale yellow oil.

### Preparation of Product 49

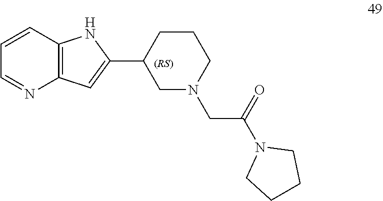 [SMILES: O=C(CN1CCCC(c2cc3ncccc3[nH]2)C1)N1CCCC1]

1-(Chloroacetyl)pyrrolidine (CAS: 20266-00-6; 48.4 mg, 0.33 mmol) was added to a stirred suspension of intermediate 5 (60 mg, 0.3 mmol) and diisopropylethylamine (0.066 mL, 0.39 mmol) in DMF (5 mL) at rt. The mixture heated at 75° C. and stirred for 4 h. The reaction mixture was diluted with NaHCO₃(aq. sat. soltn.) and DCM. The organic layer was separated, dried over MgSO₄, filtered and the filtrate was evaporated in vacuo. The crude product was purified by flash column chromatography (silica; MeOH in DCM 0/100 to 4/96). The desired fractions were collected and concentrated in vacuo. The residue thus obtained was taken up in DCM and treated with HCl 4 N in 1,4-dioxane. The solvent was evaporated and the residue was triturated with Et₂O to yield product 49 (51 mg; 48% yield) as a pale yellow solid.

### Preparation of Product 50

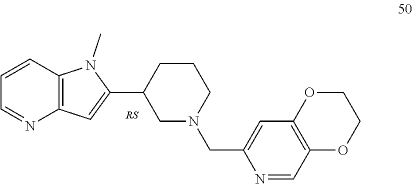 [SMILES: Cn1c(C2CCCN(Cc3cc4c(cn3)OCCO4)C2)cc2ncccc21]

A mixture of sodium cyanoborohydride (93.6 mg, 0.44 mmol), Intermediate 27 (37 mg, 0.17 mmol) and 2,3-dihydro-1,4-dioxino[2,3-c]pyridine-7-carbaldehyde (CAS: 443955-90-6; 34 mg, 0.21 mmol) in 1,2-dichloroethane (1.35 mL) in a sealed tube was heated in a microwave oven at 70° C. for 60 min. The mixture was cooled to rt. NaHCO₃(aq. sat. soltn) and DCM were added. The organic phase was separated, dried over MgSO₄, filtered and the filtrate was evaporated in vacuo. The crude product was purified by RP HPLC (Stationary phase: C₁₈ X Bridge 30×100 mm 5 μm), mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and extracted with EtOAc. The organic layer was separated, dried (MgSO₄), filtered and the solvents evaporated in vacuo to yield product 50 as yellow oil (14 mg, 22% yield).

### Preparation of Product 51

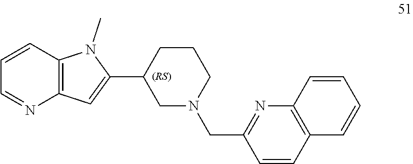 [SMILES: Cn1c(C2CCCN(Cc3ccc4ccccc4n3)C2)cc2ncccc21]

Triethylamine (0.096 mL, 0.694 mmol) was added to a stirred solution of intermediate 27 (50 mg, 0.173 mmol, bis HCl salt) in DCM (1 mL) at rt. Then 2-quinolinecarboxaldehyde (CAS: 5470-96-2; 33 mg, 0.208 mmol) followed by sodium triacetoxyborohydride (70 mg, 0.33 mmol) were added. The mixture was further stirred at rt for 25 h. The reaction mixture was diluted with NaHCO₃(aq. sat. soltn.) and DCM. The organic layer was separated, dried over MgSO₄, filtered and the filtrate was evaporated in vacuo. The residue thus obtained was purified by reverse phase HPLC (Stationary phase: C₁₈ X Bridge 30×100 mm 5 μm), mobile phase: gradient from 67% 10 mM NH₄CO₃H pH 9 solution in water, 33% CH₃CN to 50% 10 mM NH₄CO₃H pH 9 solution in water, 50% CH₃CN). The desired fractions were collected and extracted with EtOAc. The organic layer was separated, dried (MgSO₄), filtered and the solvents evaporated in vacuo to yield product 51 (18 mg, 29% yield) as a yellow oil.

### Preparation of Product 52

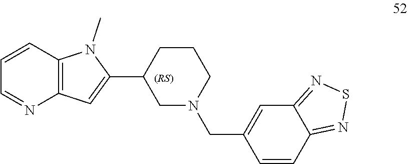 [SMILES: Cn1c(C2CCCN(Cc3ccc4nsnc4c3)C2)cc2ncccc21]

Triethylamine (0.11 mL, 0.79 mmol) was added to a stirred solution of intermediate 27 (50 mg, 0.2 mmol, HCl salt) in DCM (1.14 mL) at rt. Then 2,1,3-benzothiadiazole-5-carbaldehyde (CAS: 71605-72-6; 69 mg, 0.42 mmol) followed by sodium triacetoxyborohydride (127 mg, 0.6 mmol) were added. The mixture was further stirred at rt for 21 h. The reaction mixture was diluted with NaHCO₃(aq. sat. soltn.) and DCM. The organic layer was separated, dried over MgSO₄, filtered and the filtrate was evaporated in vacuo. The residue thus obtained was purified by reverse phase HPLC (Stationary phase: C₁₈ X Bridge 30×100 mm 5 μm), mobile phase: gradient from 60% 10 mM NH₄CO₃H pH 9 solution in water, 40% CH₃CN to 43% 10 mM NH₄CO₃H pH 9 solution in water, 57% CH₃CN). The desired fractions were collected and extracted with EtOAc. The organic layer was separated, dried (MgSO₄), filtered and the solvents evaporated in vacuo to yield product 52 (31 mg, 43% yield) as a yellow oil.

### Preparation of Product 53

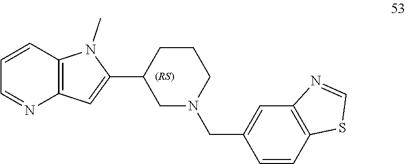 [SMILES: Cn1c(C2CCCN(Cc3ccc4scnc4c3)C2)cc2ncccc21]

Triethylamine (0.098 mL, 0.70 mmol) was added to a stirred solution of intermediate 27 (50 mg, 0.18 mmol, bis HCl salt) in DCM (0.84 mL) at rt. Then benzo[d]thiazole-5-carbaldehyde (CAS: 211915-60-7; 34.3 mg, 0.21 mmol) followed by sodium triacetoxyborohydride (56.3 mg, 0.27 mmol) were added. The mixture was further stirred at rt for 15 h. The reaction mixture was diluted with NaHCO₃(aq. sat. soltn.) and DCM. The organic layer was separated, dried over MgSO₄, filtered and the filtrate was evaporated in vacuo. The residue thus obtained was purified by reverse phase HPLC (Stationary phase: C₁₈ X Bridge 30×100 mm 5 μm), mobile phase: gradient from 74% 10 mM NH₄CO₃H pH 9 solution in water, 26% CH₃CN to 58% 10 mM NH₄CO₃H pH 9 solution in water, 42% CH₃CN). The desired fractions were collected and extracted with EtOAc. The organic layer was separated, dried (MgSO₄), filtered and the solvents evaporated in vacuo to yield product 53 (16 mg, 25% yield) as a yellow oil.

### Preparation of Product 54

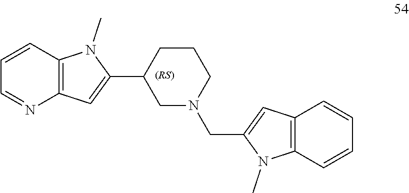 [SMILES: Cn1c(CN2CCCC(c3cc4ncccc4n3C)C2)cc2ccccc21]

Triethylamine (0.101 mL, 0.73 mmol) was added to a stirred solution of intermediate 27 (52.3 mg, 0.18 mmol, bis HCl salt) in DCM (1.05 mL) at rt. Then 1-methyl-1H-indole-2-carbaldehyde (CAS: 27421-51-8; 34.4 mg, 0.22 mmol) followed by sodium triacetoxyborohydride (84 mg, 0.4 mmol) were added. The mixture was further stirred at rt for 18 h. The reaction mixture was diluted with NaHCO₃(aq. sat. soltn.) and DCM. The organic layer was separated, dried over MgSO₄, filtered and the filtrate was evaporated in vacuo. The residue thus obtained was purified by reverse phase HPLC (Stationary phase: C₁₈ X Bridge 30×100 mm 5 μm), mobile phase: gradient from 54% 10 mM NH₄CO₃H pH 9 solution in water, 46% CH₃CN to 36% 10 mM NH₄CO₃H pH 9 solution in Water, 64% CH₃CN). The desired fractions were collected and extracted with EtOAc. The organic layer was separated, dried (MgSO₄), filtered and the solvents evaporated in vacuo to yield product 54 (45.7 mg, 70% yield) as a yellow oil.

### Preparation of Product 55

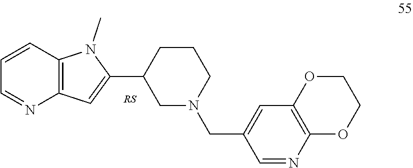 [SMILES: Cn1c(C2CCCN(Cc3cnc4c(c3)OCCO4)C2)cc2ncccc21]

A mixture of sodium triacetoxyborohydride (126 mg, 0.6 mmol), Intermediate 27 (48.1 mg, 0.22 mmol) and 2,3-dihydro-[1,4]dioxino[2,3-b]pyridine-7-carbaldehyde (CAS: 95849-26-6; 63.9 mg, 0.39 mmol) in 1,2-dichloroethane (1.3 mL) in a sealed tube was heated in a microwave oven at 70° C. for 60 min. The mixture was cooled to rt. NaHCO₃ (aq. sat. soltn) and DCM were added. The organic phase was separated, dried over MgSO₄, filtered and the filtrate was evaporated in vacuo. The crude product was purified by RP HPLC (Stationary phase: C₁₈ X Bridge 30×100 mm 5 μm), mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and extracted with EtOAc. The organic layer was separated, dried (MgSO₄), filtered and the solvents evaporated in vacuo to yield product 55 as yellow oil (26.6 mg, 32% yield).

### Preparation of Product 56

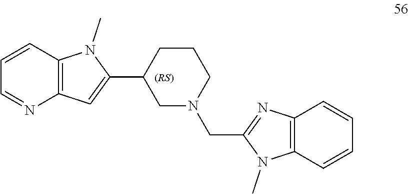 [SMILES: Cn1c(C2CCCN(Cc3nc4ccccc4n3C)C2)cc2ncccc21]

Di-isopropylethylamine (0.121 mL, 0.7 mmol) followed by 2-(chloromethyl)-1-methyl-1H-benzo[d]imidazole (CAS: 4760-35-4; 33.1 mg, 0.18 mmol) were added to a stirred suspension of intermediate 27 (50.5 mg, 0.17 mmol, bis HCl salt) in acetonitrile (0.85 mL) in a sealed tube. The mixture was stirred at room temperature for 20 h under N₂ atmosphere. The mixture was diluted with an aqueous saturated solution of NH₄Cl and extracted with EtOAc. The organic layer was separated, dried (MgSO₄), filtered and the solvents evaporated in vacuo. The crude product was purified by reverse phase HPLC (stationary phase: C₁₈ X Bridge 30×100 5 μm), mobile phase: gradient from 74% 0.1% NH₄CO₃H/NH₄OH pH 9 solution in water, 26% CH₃CN to 58% 0.1% NH₄CO₃H/NH₄OH pH 9 solution in water, 42% CH₃CN). The desired fractions were collected and concentrated in vacuo to yield product 56 (13.6 mg, 22% yield) as a yellow oil.

### Preparation of Product 57

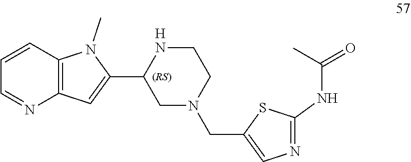

A mixture of intermediate 57 (115 mg, 0.234 mmol) and NaOH (0.247 mL, 4.69 mmol, 50% in water) in ethanol (2.2 mL) was heated at 70° C. for 6 h into a sealed tube. The ethanol was evaporated in vacuo and the resulting mixture was extracted with EtOAc. The organic layer was separated, dried (Na₂SO₄), filtered and the solvents evaporated in vacuo. The crude product was purified by reverse phase HPLC (stationary phase: C₁₈ XBridge 30×100 5 μm), mobile phase: gradient from 54% 0.1% NH₄CO₃H/NH₄OH pH 9 solution in water, 46% CH₃CN to 64% 0.1% NH₄CO₃H/NH₄OH pH 9 solution in water, 36% CH₃CN). The desired fractions were collected and concentrated in vacuo to yield product 57 (27 mg, 32% yield) as a white solid.

### Preparation of Product 58

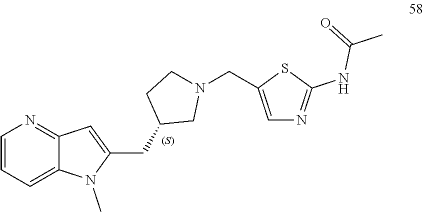

Product 58 (3-S absolute configuration) was prepared following the same reaction sequence as for the preparation of product 25 and starting from the corresponding enantiopure 3-R-iodomethylpyrrolidine-1-carboxylic acid tert-butyl ester (CAS: 1187932-69-9).

### Preparation of Product 59

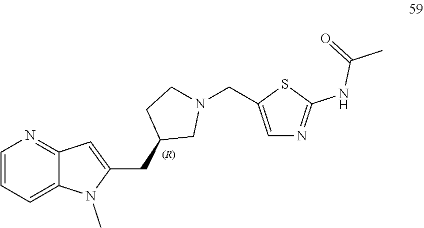

Product 59 (3-R absolute configuration) was prepared following the same reaction sequence as for the preparation of product 25 and starting from the corresponding enantiopure 3-S-iodomethylpyrrolidine-1-carboxylic acid tert-butyl ester (CAS: 224168-68-7).

### Preparation of Product 60

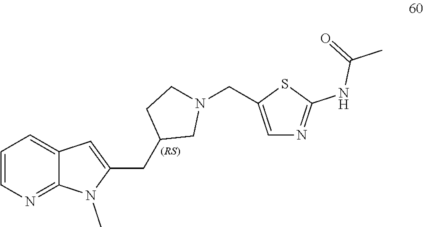 [SMILES: CC(=O)Nc1ncc(CN2CCC(Cc3cc4cccnc4n3C)C2)s1]

Sodium triacetoxyborohydride (68 mg, 0.32 mmol) was added to a stirred solution of intermediate 59 (46 mg, 0.21 mmol), intermediate 40 (44 mg, 0.26 mmol) in DCM (0.36 mL) and MeOH (0.36 mL) at rt. The mixture was further stirred at rt for 16 h. The volatiles were evaporated in vacuo and the residue thus obtained was purified by flash chromatography (silica; 7M solution of ammonia in MeOH in DCM, 0/100 to 6/94). The desired fractions were concentrated in vacuo to yield product 60 (54 mg, 68% yield) as orange solid.

### Preparation of Product 61

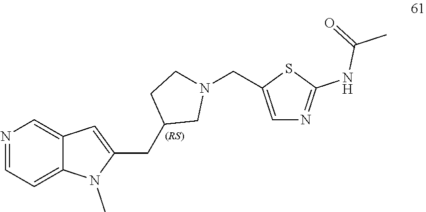 [SMILES: CC(=O)Nc1ncc(CN2CCC(Cc3cc4cnccc4n3C)C2)s1]

Sodium triacetoxyborohydride (60 mg, 0.28 mmol) was added to a stirred solution of intermediate 65 (40 mg, 0.19 mmol), intermediate 40 (38 mg, 0.22 mmol) in DCM (0.31 mL) and MeOH (0.31 mL) at rt. The mixture was further stirred at rt for 16 h. The volatiles were evaporated in vacuo and the residue thus obtained was purified by flash chromatography (silica; 7M solution of ammonia in MeOH in DCM, 0/100 to 6/94). The desired fractions were concentrated in vacuo to yield product 61 (35 mg, 51% yield) as yellow solid.

### Preparation of Product 62

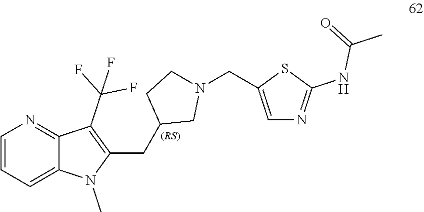 [SMILES: CC(=O)Nc1ncc(CN2CCC(Cc3c(C(F)(F)F)c4ncccc4n3C)C2)s1]

Trifluoroacetic acid (0.5 mL, 6.53 mmol) was added to a solution of intermediate 67 (17 mg, 0.044 mmol) in DCM (0.5 mL). The solution was stirred for 30 min at rt. Then the solvent was evaporated. The residue was taken up in DCM (1 mL) and acetic acid (0.05 mL) was added. Then intermediate 40 (11.32 mg, 0.067 mmol) and sodium triacetoxyborohydride (23.5 mg, 0.11 mmol) were added. The mixture was stirred at rt for 2 h. Additional sodium triacetoxyborohydride (23.5 mg, 0.11 mmol) was added. The mixture was stirred at rt overnight. Then NaHCO₃(aq. sat. soltn.) was added and the mixture was extracted with DCM. The combined organic extracts were dried (Na₂SO₄), filtered and the solvent evaporated. The residue was purified by column chromatography (silica, MeOH in EtOAc 0/100 to 50/50). Desired fractions were collected, the solvent evaporated and the residue thus obtained was further purified by RP HPLC (Stationary phase: C₁₈ X Bridge 30×100 mm 5 μm), mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo to yield product 62 (8.6 mg, 44% yield) as a foam.

### Preparation of Product 63

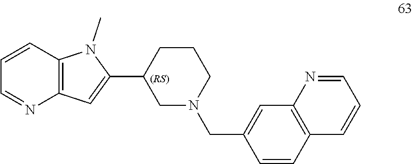 [SMILES: Cn1c(C2CCCN(Cc3ccc4cccnc4c3)C2)cc2ncccc21]

Sodium triacetoxyborohydride (408 mg, 1.93 mmol) was added to a stirred solution of intermediate 27 (41 mg, 0.19 mmol), and 6-quinolinecarboxaldehyde (CAS: 4113-04-6; 40.4 mg, 0.26 mmol) in 1,2-dichloroethane (1 mL) at rt. The mixture was stirred at rt for 26 h, diluted with MeOH and sodium cyanoborohydride (29.9 mg, 0.48 mmol) was added. The mixture was stirred at rt for a further 18h. Then NaHCO₃(aq. sat. soltn.) and DCM were added and the organic layer was separated dried (MgSO₄), filtered and the solvents evaporated in vacuo. The residue thus obtained was further purified by RP HPLC (Stationary phase: C₁₈ X Bridge 30×100 mm 5 μm), mobile phase: gradient from 74% 0.1% NH₄CO₃H/NH₄OH pH 9 solution in water, 26% CH₃CN to 58% 0.1% NH₄CO₃H/NH₄OH pH 9 solution in water, 42% CH₃CN). The desired fractions were concentrated in vacuo to yield product 63 (9.4 mg, 14% yield) as a colourless oil.

### Preparation of Product 64

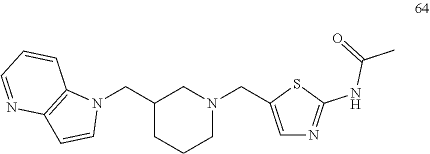 [SMILES: CC(=O)Nc1ncc(CN2CCCC(Cn3ccc4ncccc43)C2)s1]

Diisopropylethylamine (0.171 mL, 1 mmol) was added to a stirred suspension of intermediate 69 (50 mg, 0.2 mmol) in DCM (1.1 mL) at rt. The mixture was stirred at rt for 5 min and then intermediate 42 (40.5 mg, 0.283 mmol) and sodium triacetoxyborohydride (63.1 mg, 0.3 mmol) were added. The mixture was further stirred at rt for 16 h. Then NaHCO₃(aq. sat. soltn.) was added. The organic layer was separated dried (MgSO₄), filtered and the solvent evaporated. The residue thus obtained was purified by flash chromatography (silica; MeOH in DCM, 0/100 to 10/90). The desired fractions were concentrated in vacuo to yield product 64 (51.4 mg, 70% yield) as a yellow solid.

The following compounds were prepared following the methods exemplified in the Experimental Part. In case no salt form is indicated, the compound was obtained as a free base. ‘Ex. No.’ refers to the Example number according to which protocol the compound was synthesized. ‘Co. No.’ means compound number.

TABLE 1

| 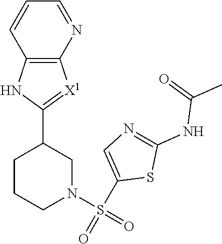 | | | |
|---|---|---|---|
| Co. | | | |
| no. | Ex no. | X¹ | Stereochemistry |
| 1 | E1 | N | 3-RS |
| 2 | E2 | CH | 3-RS |
| 3 | E2 | CH | 3-R* |
| 4 | E2 | CH | 3-S* |

TABLE 2

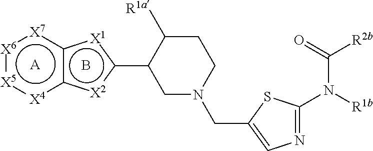

| Co. | Ex. | | | | | | | | | | |
|---|---|---|---|---|---|---|---|---|---|---|---|
| no. | no. | X¹ | X² | X⁴ | X⁵ | X⁶ | X⁷ | R^{1a′} | R^{1b} | R^{2b} | Stereo_chem |
| 5 | E3, | CH | NH | CH | CH | CH | N | H | H | —CH₃ | 3-RS |
| | E4 | | | | | | | | | | |
| 6 | E3 | CH | NH | CH | CH | CH | N | H | H | —CH₃ | 3-R* |
| 7 | E2 | CH | NH | CH | CH | CH | N | H | H | —CH₃ | 3-S* |
| 8 | E2 | CH | NH | N | CH | CH | CH | H | H | —CH₃ | 3-RS |
| 9 | E2 | CH | NH | CH | CH | CH | N | H | H | —CH₂CH₃ | 3-RS |
| 10 | E2 | CH | NH | CH | CH | CH | N | H | —CH₃ | —CH₃ | 3-RS |
| 11 | E2 | CH | NH | CH | N | CH | CH | H | H | —CH₃ | 3-RS |
| 12 | E2 | CH | NH | CH | CH | N | CH | H | H | —CH₃ | 3-RS |
| 13 | E2 | CH | NH | CH | CH | N | CH | H | H | —CH₃ | 3-R* |
| 14 | E2 | CH | NH | CH | CH | N | CH | H | H | —CH₃ | 3-S* |
| 15 | E2 | CH | NCH₃ | CH | CH | CH | N | H | H | —CH₃ | 3-RS |
| 16 | E2 | CH | NCH₃ | CH | CH | CH | N | H | H | —CH₃ | 3-R* |
| 17 | E2 | CH | NCH₃ | CH | CH | CH | N | H | H | —CH₃ | 3-S* |
| 18 | E3 | N | NH | CH | CH | CH | N | H | H | —CH₃ | 3-R* |
| 19 | E3 | N | NH | CH | CH | CH | N | H | H | —CH₃ | 3-S* |
| 20 | E3 | N | NCH₃ | CH | CH | CH | N | H | H | —CH₃ | 3-RS |
| 21 | E3 | NCH₃ | N | CH | CH | CH | N | H | H | —CH₃ | 3-RS |
| 22 | E2 | CH | NH | N | CH | CH | CH | —CH₃ | H | —CH₃ | trans, 3-RS- |
| | | | | | | | | | | | 4-RS |
| 23 | E2 | CH | NH | N | CH | CH | CH | —CH₃ | H | —CH₃ | cis, 3-RS- |
| | | | | | | | | | | | 4-RS |

TABLE 3

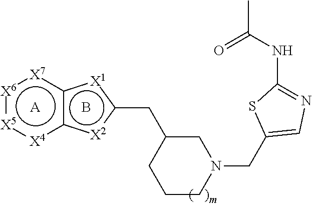

| Co. | Ex. | | | | | | | | |
|---|---|---|---|---|---|---|---|---|---|
| no. | no. | X¹ | X² | X⁴ | X⁵ | X⁶ | X⁷ | m | Stereo_chem |
| 24 | E2 | CH | NH | CH | CH | CH | N | 0 | 3-RS |
| 25 | E2 | CH | NCH₃ | CH | CH | CH | N | 0 | 3-RS |
| 26 | E2 | CH | NCH₃ | CH | CH | CH | N | 1 | 3-RS |
| 58 | E2 | CH | NCH₃ | CH | CH | CH | N | 0 | 3-S |
| 59 | E2 | CH | NCH₃ | CH | CH | CH | N | 0 | 3-R |
| 60 | E2 | CH | NCH₃ | N | CH | CH | CH | 0 | 3-RS |
| 61 | E2 | CH | NCH₃ | CH | CH | N | CH | 0 | 3-RS |
| 62 | E2 | CCF₃ | NCH₃ | CH | CH | CH | N | 0 | 3-RS |

TABLE 4

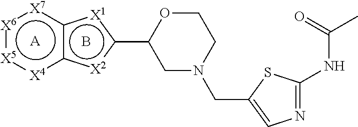

| Co. | Ex. | | | | | | | |
|---|---|---|---|---|---|---|---|---|
| no. | no. | X¹ | X² | X⁴ | X⁵ | X⁶ | X⁷ | Stereochem |
| 27 | E2 | CH | NH | CH | CH | CH | N | 3-RS |

TABLE 5

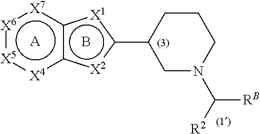

| Co. | Ex. | | | | | | | | | |
|---|---|---|---|---|---|---|---|---|---|---|
| no. | no. | X¹ | X² | X⁴ | X⁵ | X⁶ | X⁷ | R² | R^{B} | Stereochem |
| 28 | E4 | CH | NH | CH | CH | CH | N | —CH₃ | b-2 | 1′-RS-3-RS |
| 29 | E4 | CH | NH | CH | CH | CH | N | —CH₃ | b-2 | 1′-R*-3-R* |
| 30 | E4 | CH | NH | CH | CH | CH | N | —CH₃ | b-2 | 1′-S*-3-R* |
| 31 | E4 | CH | NH | CH | CH | CH | N | —CH₃ | b-2 | 1′-R*-3-S* |
| 32 | E4 | CH | NH | CH | CH | CH | N | —CH₃ | b-2 | 1′-S*-3-S* |
| 33 | E2, | CH | NH | CH | CH | CH | N | —CH₃ | b-3 | 3-RS |
| | E4 | | | | | | | | | |
| 34 | E2, | CH | NH | CH | CH | CH | N | —CH₃ | b-3 | 1′-R*-3-R* |
| | E4 | | | | | | | | | |
| 35 | E2, | CH | NH | CH | CH | CH | N | —CH₃ | b-3 | 1′-S*-3-R* |
| | E4 | | | | | | | | | |
| 36 | E2, | CH | NH | CH | CH | CH | N | —CH₃ | b-3 | 1′-R*-3-S* |
| | E4 | | | | | | | | | |
| 37 | E2, | CH | NH | CH | CH | CH | N | —CH₃ | b-3 | 1′-S*-3-S* |
| | E4 | | | | | | | | | |
| 38 | E2 | CH | NH | CH | CH | CH | N | —H | b-2 | 3-RS |
| 39^{&} | E2 | CH | NH | CH | CH | CH | N | —H | b-3 | 3-RS |
| 40 | E2 | CH | NCH₃ | CH | CH | CH | N | —CH₃ | b-3 | 1′-RS-3-RS |
| 41 | E2 | CH | NCH₃ | CH | CH | CH | N | —H | b-4 | 3-RS |
| 42 | E2 | CH | NCH₃ | CH | CH | CH | N | —H | b-4 | 3-R* |
| 43 | E2 | CH | NCH₃ | CH | CH | CH | N | —H | b-4 | 3-S* |
| 44 | E6 | CH | NCH₃ | CH | CH | CH | N | —CH₃ | b-2 | 1′-RS-3-RS |
| 45 | E6 | CH | NCH₃ | CH | CH | CH | N | —CH₃ | b-2 | 1′-R*-3-R* |
| 46 | E6 | CH | NCH₃ | CH | CH | CH | N | —CH₃ | b-2 | 1′-S*-3-R* |
| 47 | E6 | CH | NCH₃ | CH | CH | CH | N | —CH₃ | b-2 | 1′-R*-3-S* |
| 48 | E6 | CH | NCH₃ | CH | CH | CH | N | —CH₃ | b-2 | 1′-S*-3-S* |
| 49 | E3 | CH | NH | CH | CH | CH | N | —H | b-12 | 3-RS |
| 50 | E2 | CH | NCH₃ | CH | CH | CH | N | —CH₃ | b-5 | 3-RS |
| 51 | E2 | CH | NCH₃ | CH | CH | CH | N | —CH₃ | b-6 | 3-RS |
| 52 | E2 | CH | NCH₃ | CH | CH | CH | N | —CH₃ | b-7 | 3-RS |
| 53 | E2 | CH | NCH₃ | CH | CH | CH | N | —CH₃ | b-11; | 3-RS |
| | | | | | | | | | R^{4b} is H | |
| 54 | E2 | CH | NCH₃ | CH | CH | CH | N | —CH₃ | b-9; R^{3b} | 3-RS |
| | | | | | | | | | is CH³ | |
| | | | | | | | | | and Q¹ is | |
| | | | | | | | | | CH | |
| 55 | E2 | CH | NCH₃ | CH | CH | CH | N | —CH₃ | b-8 | 3-RS |
| 56 | E3 | CH | NCH₃ | CH | CH | CH | N | —CH₃ | b-9; R^{3b} | 3-RS |
| | | | | | | | | | is CH₃ | |
| | | | | | | | | | and Q¹ is | |
| | | | | | | | | | N | |
| 63 | E2 | CH | NCH₃ | CH | CH | CH | N | —CH₃ | b-10 | 3-RS |

^{&}means •HCl salt

TABLE 6

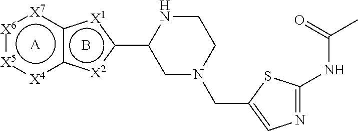

| Co. | | | | | | | | |
|---|---|---|---|---|---|---|---|---|
| no. | Ex. no. | X¹ | X² | X⁴ | X⁵ | X⁶ | X⁷ | Stereochem |
| 57 | E2 followed by | CH | NH | CH | CH | CH | N | 3-RS |
| | standard Cbz | | | | | | | |
| | protecting | | | | | | | |
| | group cleavage | | | | | | | |

TABLE 7

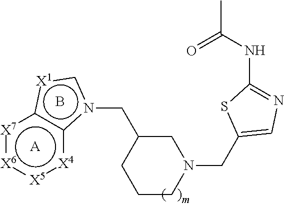

| Co. | | | | | | | | |
|---|---|---|---|---|---|---|---|---|
| no. | Ex. no. | X¹ | X⁴ | X⁵ | X⁶ | X⁷ | m | Stereochem |
| 64 | E37 | CH | CH | CH | CH | N | 1 | 3-RS |

### C. Analytical Part

### Melting Points:

Values are peak values, and are obtained with experimental uncertainties that are commonly associated with this analytical method.

DSC823e (A): For a number of compounds, melting points were determined with a DSC823e (Mettler-Toledo) apparatus. Melting points were measured with a temperature gradient of 10° C./minute. Maximum temperature was 300° C. Values are peak values. Mettler Toledo Mettler FP 81HT/FP90 apparatus (B) or Mettler Toledo MP50 (C): For a number of compounds, melting points were determined in open capillary tubes on a Mettler FP 81HT/FP90 apparatus. Melting points were measured with a temperature gradient of 1, 3, 5 or 10° C./minute. Maximum temperature was 300° C. The melting point was read from a digital display.

### LCMS

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl.), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, “CSH” charged surface hybrid, “UPLC” Ultra Performance Liquid Chromatography, “DAD” Diode Array Detector.

TABLE 8
LC-MS Methods (Flow expressed in mL/min; column temperature (T) in ° C.;
Run time in min).

| | | | | | Flow | |
|---|---|---|---|---|---|---|
| | | | | | ------ | Run |
| Method | Instrument | Column | Mobile Phase | Gradient | Col T | Time |
| 1 | Agilent: | Agilent: | A: 95% | From 95% A | 1 | 7 |
| | HP1100- | Eclipse Plus | CH₃COONH₄ | to 0% A in | ------ | |
| | DAD, MSD | C18 | 6.5 mM + | 5.0 min, held | 60 | |
| | G1956B | (3.5 μm, | 5% CH₃CN, | for 0.15 min, | | |
| | | 2.1 × 30 mm) | B: CH₃CN | back to 95% A | | |
| | | | | in 0.15 min, | | |
| | | | | held for 1.7 min | | |
| 2 | Waters: | Waters: | A: 95% | From 95% A | 1 | 5 |
| | Acquity ® | BEH C18 | CH₃COONH₄ | to 5% A in | ------ | |
| | UPLC ®- | (1.7 μm, | 6.5 mM + | 4.6 min, held | 50 | |
| | DAD/SQD | 2.1 × 50 mm) | 5% CH₃CN, | for 0.4 min | | |
| | | | B: CH₃CN | | | |
| 3 | Waters: | Waters: | A: 95% | From 95% A | 1 | 5 |
| | Acquity ® | BEHC18 | CH₃COONH₄ | to 5% A in | ------ | |
| | IClass | (1.7 μm, | 6.5 mM + | 4.6 min, held | 50 | |
| | UPLC ®- | 2.1 × 50 mm) | 5% CH₃CN, | for 0.4 min | | |
| | DAD/Xevo | | B: CH₃CN | | | |
| | G2-S | | | | | |
| | QTOF | | | | | |
| 4 | Agilent | YMC-pack | A: 0.1% | From 95% A | 2.6 | 6.2 |
| | 1100 HPLC | ODS-AQ | HCOOH in | to 5% A in 4.8 | ------ | |
| | DAD | C18 (3 μm | H₂O | min, held for | 35 | |
| | LC/MS | 50 × 4.6 mm) | B: CH₃CN | 1.0 min, to | | |
| | G1956A | | | 95% A in 0.2 min | | |
| 5 | Agilent | YMC-pack | A: 0.1% | ISET 2V1.0 | 2.6 | 6.0 |
| | 1290 | ODS-AQ | HCOOH in | Emulated | ------ | |
| | Infinity | C18 (3 μm | H₂O | Agilent Pump | 35 | |
| | DAD TOF- | 50 × 4.6 mm) | B: CH₃CN | G1312AV1.0 | | |
| | LC/MS | | | From 94.51% | | |
| | G6224A | | | A to 5% A in | | |
| | | | | 4.8 min, held | | |
| | | | | for 1.0 min, to | | |
| | | | | 95% A in 0.2 | | |
| | | | | min | | |
| 6 | Waters: | Waters: | A: 95% | 84.2% A for | 0.34 | 6.2 |
| | Acquity | BEH C18 | CH₃COONH₄ | 0.5 min, to | ------ | |
| | UPLC ®- | (1.7 μm, | 7 mM + | 10.5% A in 2.2 | 40 | |
| | DAD/ | 2.1 × 100 mm) | 5% CH₃CN | min, held for | | |
| | Quattro | | to 84.2% Am | 1.9 min, back | | |
| | Micro ™ | | B: CH₃CN | 0.7 min, held | | |
| | | | | for 0.7 min. | | |
| 7 | Waters: | Waters: | A: 95% | From 84.2% A | 0.34 | 6.1 |
| | Acquity | BEHC18 | CH3COONH₄ | to 10.5% A in | ------ | |
| | UPLC ® | (1.7 μm, | 7 mM + | 2.2 min, held | 40 | |
| | H-Class- | 2.1 × 100 mm) | 5% CH₃CN | for 1.9 min, | | |
| | DAD/SQD2 | | B: CH₃CN | back to 84.2% | | |
| | | | | A in 0.7 min, | | |
| | | | | held for 0.7 min. | | |
| 8 | Waters: | Waters: | A: 95% | From 95% A | 1 | 2 |
| | Acquity ® | BEH C18 | CH3COONH₄ | to 40% A in | ------ | |
| | UPLC ®- | (1.7 μm, 2.1 × 50 mm) | 6.5 mM + | 1.2 min, to 5% | 50 | |
| | DAD/SQD | | 5% CH₃CN, | A in 0.6 min | | |
| | | | B: CH₃CN | held for | | |
| | | | | 0.2 min | | |

(*) Different MS tuning parameters due to low sensitivity

TABLE 9
Analytical data - melting point (M.p.) and LCMS: [M + H]⁺ means the
protonated mass of the free base of the compound, [M − H]⁻ means the
deprotonated mass of the free base of the compound or the type of adduct
specified [M + CH₃COO]⁻). Rₜ means retention time (in min).
For some compounds, exact mass was determined.

| Co. | | | | LCMS |
|---|---|---|---|---|
| No. | M.p. (° C.) | [M + H]⁺ | Rₜ | Method |
| 1 | n.d. | 407 | 1.97 | 1 |
| 2 | n.d. | 406 | 2.17 | 6 |
| 3 | n.d. | 406 | 2.17 | 6 |
| 4 | n.d. | 406 | 2.17 | 6 |
| 5 | n.d. | 356 | 0.95 | 4 |
| 6 | n.d. | 356 | 1.93 | 6 |
| 7 | n.d. | 356 | 1.92 | 6 |
| 8 | n.d. | 356 | 1.52 | 3 |
| 9 | 235.0° C. (C) | 370 | 1.67 | 4 |
| 10 | n.d. | 370 | 0.86 | 4 |
| 11 | n.d. | 356 | 1.38 | 3 |
| 12 | n.d. | 356 | 1.13 | 3 |
| 13 | n.d. | 356 | 1.78 | 6 |
| 14 | n.d. | 356 | 1.79 | 6 |
| 15 | 214.8° C. (C) | 370 | 1.25 | 3 |
| 16 | n.d. | 370 | 2.10 | 6 |
| 17 | n.d. | 370 | 2.10 | 6 |
| 18 | 223.94° C. (A) | 357 | 0.81 | 3 |
| 19 | 228.20° C. (A) | 357 | 0.83 | 3 |
| 20 | n.d. | 371 | 0.85 | 3 |
| 21 | n.d. | 371 | 1.01 | 3 |
| 22 | n.d. | 370 | 1.15 | 3 |
| 23 | n.d. | 370 | 1.51 | 3 |
| 24 | n.d. | 356 | 1.04 | 3 |
| 25 | n.d. | 370 | 1.07 | 3 |
| 26 | n.d. | 384 | 1.34 | 3 |
| 27 | n.d. | 358 | 0.86 | 3 |
| 28 | n.d. | 350 | 1.15 | 4 |
| 29 | n.d. | 350 | 1.58 | 3 |
| 30 | n.d. | 350 | 1.62 | 3 |
| 31 | n.d. | 350 | 1.61 | 3 |
| 32 | n.d. | 350 | 1.58 | 3 |
| 33 | n.d. | 364 | 1.16 | 4 |
| 34 | n.d. | 364 | 1.49 | 3 |
| 35 | n.d. | 364 | 1.49 | 3 |
| 36 | n.d. | 364 | 1.51 | 3 |
| 37 | n.d. | 364 | 1.49 | 3 |
| 38 | n.d. | 336 | 1.10 | 4 |
| 39 | 178.2° C. (C) | 350 | 1.23 | 4 |
| 40 | n.d. | 378 | 1.76 | 3 |
| 41 | n.d. | 358 | 1.56 | 3 |
| 42 | n.d. | 358 | 2.27 | 7 |
| 43 | n.d. | 358 | 2.25 | 7 |
| 44 | n.d. | 364 | 1.21 | 4 |
| 45 | n.d. | 364 | 1.85 | 3 |
| 46 | n.d. | 364 | 1.84 | 3 |
| 47 | n.d. | 364 | 1.84 | 3 |
| 48 | n.d. | 364 | 1.85 | 3 |
| 49 | 179.9° C. (C) | 313 | 1.19 | 5 |
| 50 | n.d. | 365 | 1.52 | 3 |
| 51 | n.d. | 357 | 1.93 | 3 |
| 52 | n.d. | 364 | 2.09 | 3 |
| 53 | n.d. | 363 | 1.71 | 3 |
| 54 | n.d. | 359 | 2.69 | 3 |
| 55 | n.d. | 365 | 1.49 | 3 |
| 56 | n.d. | 360 | 1.91 | 3 |
| 57 | n.d. | 357 | 0.66 | 3 |
| 58 | 187.16° C. (A) | 370 | 1.05 | 3 |
| 59 | 190.09° C. (A)* | 370 | 1.03 | 3 |
| 60 | 153.79° C. (A) | 370 | 1.33 | 3 |
| 61 | 195.06° C. (A) | 370 | 0.97 | 3 |
| 62 | n.d. | 438 | 1.41 | 3 |
| 63 | n.d. | 357 | 1.67 | 3 |
| 64 | n.d. | 370 | 1.26 | 3 |

n.d. means not determined.

### Optical Rotations

Optical rotations were measured on a Perkin-Elmer 341 polarimeter with a sodium lamp and reported as follows: [α]° (λ, g/100 ml, solvent, T ° C.).

[α]_{λ}^{T}=(100σ)/(l×c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation the concentration and solvent are always provided in parentheses after the rotation. The rotation is reported using degrees and no units of concentration are given (it is assumed to be g/100 mL).

TABLE 10
Optical Rotation data.

| | | Wavelength | Concentration | | |
|---|---|---|---|---|---|
| Co. No. | α_{D} (°) | (nm) | w/v % | Solvent | Temp. (° C.) |
| 7 | +241.2 | 589 | 0.40 | DMF | 20 |
| 13 | +10.7 | 589 | 0.50 | DMF | 20 |
| 14 | −9.87 | 589 | 0.50 | DMF | 20 |
| 16 | +82.5 | 589 | 0.52 | DMF | 20 |
| 17 | −90.9 | 589 | 0.51 | DMF | 20 |
| 18 | +116.7 | 589 | 0.50 | DMF | 20 |
| 19 | −107.4 | 589 | 0.54 | DMF | 20 |
| 29 | −5.32 | 589 | 0.49 | DMF | 20 |
| 30 | −1.12 | 589 | 0.52 | DMF | 20 |
| 31 | +0.22 | 589 | 0.53 | DMF | 20 |
| 32 | +4.39 | 589 | 0.55 | DMF | 20 |
| 34 | −26.9 | 589 | 0.71 | DMF | 20 |
| 35 | −1.70 | 589 | 0.78 | DMF | 20 |
| 36 | +0.7 | 589 | 0.78 | DMF | 20 |
| 37 | +27.2 | 589 | 0.72 | DMF | 20 |
| 42 | +105.2 | 589 | 0.40 | DMF | 20 |
| 43 | −82.7 | 589 | 0.40 | DMF | 20 |
| 45 | −6.2 | 589 | 0.64 | DMF | 20 |
| 47 | −42.9 | 589 | 0.67 | DMF | 20 |
| 48 | +3.0 | 589 | 0.62 | DMF | 20 |
| 58 | +25.4 | 589 | 0.32 | DMF | 20 |
| 59 | −27.7 | 589 | 0.36 | DMF | 20 |

n.d.*: not available data due to limited solubility

### SFCMS-Methods

### General Procedure

The SFC measurement was performed using Analytical Supercritical fluid chromatography (SFC) system composed by a binary pump for delivering carbon dioxide (CO₂) and modifier, an autosampler, a columns oven with switching valve for column heating from room temperature to 80° C., a diode array detector equipped with a high-pressure flow cell standing up to 400 bars. Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 11
Analytical SFC-MS Methods (Flow expressed in mL/min; column temperature
(T) in ° C.; Backpressure in bars).

| | | | | Flow | Run time |
|---|---|---|---|---|---|
| Method | Column | Mobile Phase | Gradient | T | BPR |
| 1 | Daicel Chiralpak ® | A: CO₂ | 50% B | 3.5 | 3.0 |
| | AD-3 column (3 μm, | B: MeOH | hold 3 min, | 35 | 105 |
| | 100 × 4.6 mm) | (+0.3% iPrNH2) | | | |
| 2 | Daicel Chiralpak ® | A: CO₂ | 40% B | 3.5 | 3.0 |
| | AD-3 column (3 μm, | B: EtOH | hold 3 min, | 35 | 105 |
| | 100 × 4.6 mm) | (+0.3% iPrNH₂) | | | |
| 3 | Daicel Chiralpak ® | A: CO₂ | 10% B | 3.5 | 7.0 |
| | AS-3 column (3 μm, | B: EtOH/iPrOH | hold 3 min, | 35 | 105 |
| | 100 × 4.6 mm) | 50/50 (+0.3% | | | |
| | | iPrNH₂) | | | |
| 4 | Daicel Chiralpak ® | A: CO₂ | 10% B | 3.5 | 3.0 |
| | AS-3 column (3 μm, | B: MeOH | hold 3 min, | 35 | 105 |
| | 100 × 4.6 mm) | (+0.3% iPrNH₂) | | | |
| 5 | Daicel Chiralcel ® | A: CO₂ | 20% B | 3.5 | 3.0 |
| | OJ-3 column (3 μm, | B: MeOH | hold 3 min, | 35 | 105 |
| | 100 × 4.6 mm) | (+0.3% iPrNH₂) | | | |
| 6 | Daicel Chiralpak ® | A: CO₂ | 30% B | 3.5 | 3.0 |
| | AD-3 column (3 μm, | B: iPOH | hold 3 min, | 35 | 105 |
| | 100 × 4.6 mm) | (+0.3% iPrNH₂) | | | |
| 7 | Daicel Chiralpak | A: CO₂ | 40% B | 3.5 | 6.0 |
| | AD-3 column (3 μm, | B: EtOH | hold 6 min, | 35 | 105 |
| | 100 × 4.6 mm) | (+0.3% iPrNH₂) | | | |
| 8 | Daicel Chiralpak ® | A: CO₂ | 20% B | 3.5 | 6.0 |
| | AS-3 column (3 μm, | B: iPOH | hold 6 min, | 35 | 105 |
| | 100 × 4.6 mm) | (+0.3% iPrNH₂) | | | |

TABLE 12
Analytical SFC data—Rₜ means retention time (in minutes), [M + H]⁺
means the protonated mass of the compound, method refers to the method
used for (SFC)MS analysis of enantiomerically pure compounds.

| | | | | | Isomer Elution |
|---|---|---|---|---|---|
| Co. No. | Rₜ | [M + H]⁺ | UV Area % | Method | Order |
| 3 | 2.12 | 406 | 100 | 2 | A |
| 4 | 3.43 | 406 | 99.60 | 2 | B |
| 6 | 1.90 | 356 | 99.9 | 1 | B |
| 7 | 1.07 | 356 | 100 | 1 | A |
| 13 | 1.47 | 356 | 100 | 7 | A |
| 14 | 2.05 | 356 | 99.72 | 7 | B |
| 16 | 0.92 | 370 | 100 | 4 | B |
| 17 | 0.71 | 370 | 100 | 4 | A |
| 29 | 4.20 | 350 | 100 | 3 | A |
| 30 | 5.00 | 350 | 100 | 3 | B |
| 31 | 6.40 | 350 | 100 | 3 | C |
| 32 | 8.19 | 350 | 100 | 3 | D |
| 34 | 1.77 | 364 | 100 | 8 | A |
| 35 | 2.16 | 364 | 98.81 | 8 | B |
| 36 | 2.65 | 364 | 98.65 | 8 | C |
| 37 | 3.06 | 364 | 97.41 | 8 | D |
| 42 | 1.1 | 358 | 99.85 | 5 | A |
| 43 | 1.49 | 358 | 99.67 | 5 | B |
| 45 | 1.16 | 364 | 99.48 | 6 | B |
| 46 | 0.97 | 364 | 100 | 6 | A |
| 47 | 1.35 | 364 | 98.61 | 6 | C |
| 48 | 1.62 | 364 | 97.5 | 6 | D |

### NMR

For a number of compounds, ¹H NMR spectra were recorded on a Bruker Avance III with a 300 MHz Ultrashield magnet, on a Bruker DPX-400 spectrometer operating at 400 MHz, on a Bruker Avance I operating at 500 MHz, on a Bruker DPX-360 operating at 360 MHz, or on a Bruker Avance 600 spectrometer operating at 600 MHz, using CHLOROFORM-d (deuterated chloroform, CDCl₃) or DMSO-d₆ (deuterated DMSO, dimethyl-d₆ sulfoxide) as solvent. Chemical shifts (□) are reported in parts per million (ppm) relative to tetramethylsilane (TMS), which was used as internal standard.

TABLE 13
¹H NMR results

| Co. | |
|---|---|
| No. | ¹H NMR result |
| 1 | ¹H NMR (500 MHz, DMSO-d₆) δ ppm 1.08 (br s, 1 H) 1.55-1.76 (m, 2 H) |
| | 1.92 (br d, J = 11.0 Hz, 1 H) 2.08-2.16 (m, 1 H) 2.20 (s, 3 H) 2.73 (br t, J = 11.1 Hz, 1 |
| | H) 3.14-3.25 (m, 1 H) 3.63 (br d, J = 11.3 Hz, 1 H) 3.95 (br dd, J = 11.3, 3.8 Hz, 1 |
| | H) 7.18 (br dd, J = 7.8, 4.9 Hz, 1 H) 7.84 (br d, J = 7.5 Hz, 0.35 H) 7.95 (br d, J = 7.8 Hz, |
| | 0.65 H) 8.01 (s, 1 H) 8.24 (br d, J = 4.3 Hz, 0.65 H) 8.32 (br d, J = 4.0 Hz, 0.35 |
| | H) 12.34-13.17 (m, 2 H). |
| 2 | ¹H NMR (500 MHz, DMSO-d₆) δ ppm 1.54-1.73 (m, 2 H) 1.80-1.90 (m, 1 H) |
| | 1.94-2.02 (m, 1 H) 2.05 (s, 3 H) 2.42-2.55 (m, 2 H) 3.03-3.13 (m, 1 H) |
| | 3.55 (br d, J = 11.7 Hz, 1 H) 3.76 (br d, J = 8.5 Hz, 1 H) 6.36 (s, 1 H) 7.02 (dd, J = 8.2, 4.7 Hz, |
| | 1 H) 7.64 (d, J = 8.2 Hz, 1 H) 7.79 (br s, 1 H) 8.24 (d, J = 4.1 Hz, 1 H) 11.31 (s, |
| | 1 H) 12.73 (s, 1 H). |
| 6 | ¹H NMR (500 MHz, DMSO-d₆) δ ppm 1.49 (qd, J = 12.1, 3.5 Hz, 1 H) 1.60 (qt, |
| | J = 12.1, 3.6 Hz, 1 H) 1.70-1.77 (m, 1 H) 1.97-2.08 (m, 2 H) 2.08-2.17 (m, 4 H) |
| | 2.86 (br d, J = 10.7 Hz, 1 H) 2.97 (tt, J = 10.8, 3.6 Hz, 1 H) 3.07 (br d, J = 10.4 Hz, 1 |
| | H) 3.69 (d, J = 1.4 Hz, 2 H) 6.28 (d, J = 1.7 Hz, 1 H) 6.98 (dd, J = 8.1, 4.6 Hz, 1 H) |
| | 7.26 (s, 1 H) 7.60 (dt, J = 8.1, 1.2 Hz, 1 H) 8.21 (dd, J = 4.6, 1.4 Hz, 1 H) 11.14 (s, 1 |
| | H) 11.94 (br s, 1 H). |
| 7 | ¹H NMR (400 MHz, DMSO-d₆) δ ppm 1.43-1.54 (m, 1 H) 1.60 (q, J = 12.3 Hz, 1 |
| | H) 1.70-1.78 (m, 1 H) 1.97-2.08 (m, 2 H) 2.08-2.17 (m, 4 H) 2.86 (br d, |
| | J = 11.1 Hz, 1 H) 2.91-3.02 (m, 1 H) 3.07 (br d, J = 10.9 Hz, 1 H) 3.63-3.75 (m, 2 |
| | H) 6.28 (d, J = 1.6 Hz, 1 H) 6.98 (dd, J = 8.1, 4.6 Hz, 1 H) 7.26 (s, 1 H) |
| | 7.58-7.62 (m, 1 H) 8.21 (dd, J = 4.6, 1.6 Hz, 1 H) 11.16 (s, 1 H) 11.95 (br s, 1 H). |
| 8 | ¹H NMR (400 MHz, DMSO-d₆) δ ppm 1.41-1.67 (m, 2 H) 1.68-1.79 (m, 1 H) |
| | 1.96-2.06 (m, 2 H) 2.11 (s, 4 H) 2.85 (br d, J = 10.9 Hz, 1 H) 2.94 (tt, J = 11.0, 3.5 Hz, |
| | 1 H) 3.08 (br d, J = 10.4 Hz, 1 H) 3.68 (s, 2 H) 6.15 (d, J = 1.6 Hz, 1 H) |
| | 6.97 (dd, J = 7.7, 4.7 Hz, 1 H) 7.25 (s, 1 H) 7.78 (dd, J = 7.7, 1.0 Hz, 1 H) 8.09 (dd, |
| | J = 4.6, 1.6 Hz, 1 H) 11.47 (s, 1 H) 11.94 (s, 1 H). |
| 9 | ¹H NMR (300 MHz, CDCl₃) δ ppm 1.30 (t, J = 7.6 Hz, 3 H) 1.55-1.93 (m, 4 H) |
| | 2.35-2.48 (m, 1 H) 2.53 (q, J = 7.6 Hz, 2 H) 2.67 (br d, J = 9.9 Hz, 1 H) |
| | 2.79-3.03 (m, 2 H) 3.21-3.33 (m, 1 H) 3.63-3.84 (m, 2 H) 6.42 (s, 1 H) 7.06 (dd, J = 8.0, |
| | 4.7 Hz, 1 H) 7.21 (s, 1 H) 7.76 (d, J = 8.1 Hz, 1 H) 8.38 (d, J = 3.8 Hz, 1 H) 9.81 (br |
| | s, 1 H) 10.58 (br s, 1 H). |
| 10 | ¹H NMR (300 MHz, CDCl₃) δ ppm 1.55-1.98 (m, 5 H) 2.42 (s, 3 H) 2.65 (br d, |
| | J = 11.4 Hz, 1 H) 2.77-3.02 (m, 3 H) 3.21-3.33 (m, 1 H) 3.50-3.72 (m, 4 H) |
| | 3.73-3.82 (m, 1 H) 6.40 (s, 0.8 H) 6.42 (s, 0.2 H) 6.94 (s, 0.2 H) 7.28 (s, 0.8 H) |
| | 7.59 (d, J = 8.0 Hz, 0.2 H) 7.74 (d, J = 8.1 Hz, 0.8 H) 8.33-8.43 (m, 1 H) 10.06 (br |
| | s, 1 H). |
| 11 | ¹H NMR (500 MHz, CDCl₃) δ ppm 1.61-1.69 (m, 1 H) 1.77 (dt, J = 8.7, 4.3 Hz, 2 |
| | H) 1.93 (br s, 1 H) 2.27 (s, 3 H) 2.43-2.91 (m, 3 H) 2.74-2.83 (m, 1 H) 3.23 (br |
| | s, 1 H) 3.65-3.72 (m, 1 H) 3.72-3.82 (m, 1 H) 6.23 (s, 1 H) 7.20 (s, 1 H) |
| | 7.42 (dd, J = 5.6, 1.0 Hz, 1 H) 8.17 (d, J = 5.5 Hz, 1 H) 8.80 (s, 1 H) 10.36 (br s, 1 H) |
| | 11.68 (br s, 1 H). |
| 12 | ¹H NMR (500 MHz, CDCl₃) δ ppm 1.60-1.68 (m, 1 H) 1.72-1.79 (m, 2 H) |
| | 1.87 (br d, J = 11.3 Hz, 1 H) 2.29 (s, 3 H) 2.44 (br s, 1 H) 2.69 (br s, 1 H) 2.85 (br s, 2 |
| | H) 3.23 (br s, 1 H) 3.61-3.72 (m, 1 H) 3.73-3.82 (m, 1 H) 6.28 (s, 1 H) 7.21 (s, |
| | 1 H) 7.38 (d, J = 5.5 Hz, 1 H) 8.25 (d, J = 5.8 Hz, 1 H) 8.81 (s, 1 H) 10.06 (br s, 1 H) |
| | 11.28 (br s, 1H). |
| 15 | ¹H NMR (400 MHz, CDCl₃) δ ppm 1.51 (qd, J = 12.3, 4.6 Hz, 1 H) 1.71-1.90 (m, |
| | 2 H) 2.03-2.20 (m, 3 H) 2.31 (s, 3 H) 2.97-3.18 (m, 3 H) 3.69 (s, 3 H) |
| | 3.67-3.83 (m, 2 H) 6.44 (s, 1 H) 7.05 (dd, J = 8.3, 4.6 Hz, 1 H) 7.20 (s, 1 H) 7.52 (d, |
| | J = 8.1 Hz, 1 H) 8.38 (dd, J = 4.6, 1.4 Hz, 1 H) 12.37 (br s, 1 H). |
| 16 | ¹H NMR (500 MHz, CDCl₃) δ ppm 1.51 (qd, J = 12.5, 4.5 Hz, 1 H) 1.73-1.87 (m, |
| | 2 H) 2.04-2.19 (m, 3 H) 2.30 (s, 3 H) 2.99-3.09 (m, 2 H) 3.12 (br dd, J = 11.0, |
| | 1.4 Hz, 1 H) 3.69-3.79 (m, 2 H) 6.44 (s, 1 H) 7.04 (dd, J = 8.2, 4.8 Hz, 1 H) |
| | 7.20 (s, 1 H) 7.52 (d, J = 8.1 Hz, 1 H) 8.38 (dd, J = 4.6, 1.4 Hz, 1 H) 11.63 (br s, 1 H). |
| 17 | ¹H NMR (400 MHz, CDCl₃) δ ppm 1.51 (qd, J = 12.3, 4.7 Hz, 1 H) 1.69-1.89 (m, |
| | 2 H) 2.02-2.18 (m, 3 H) 2.30 (s, 3 H) 2.98-3.17 (m, 3 H) 3.69 (s, 3 H) |
| | 3.68-3.83 (m, 2 H) 6.44 (s, 1 H) 7.05 (dd, J = 8.3, 4.6 Hz, 1 H) 7.20 (s, 1 H) 7.52 (d, |
| | J = 8.1 Hz, 1 H) 8.39 (dd, J = 4.7, 1.3 Hz, 1 H). |
| 18 | ¹H NMR (500 MHz, DMSO-d₆) δ ppm 1.51-1.67 (m, 2 H) 1.69-1.81 (m, 1 H) |
| | 1.99-2.08 (m, 2 H) 2.11 (s, 3 H) 2.25 (br t, J = 10.8 Hz, 1 H) 2.87 (br d, J = 10.7 Hz, |
| | 1 H) 3.01-3.08 (m, 1 H) 3.13 (br d, J = 11.0 Hz, 1 H) 3.63-3.78 (m, 2 H) |
| | 7.10-7.18 (m, 1 H) 7.26 (s, 1 H) 7.86 (dd, J = 7.8, 1.2 Hz, 1 H) 8.23 (dd, J = 4.6, 1.4 Hz, |
| | 1 H) 12.27 (br s, 2 H). |
| 19 | ¹H NMR (500 MHz, DMSO-d₆) δ ppm 1.50-1.67 (m, 2 H) 1.69-1.83 (m, 1 H) |
| | 1.99-2.09 (m, 2 H) 2.11 (s, 3 H) 2.25 (br t, J = 10.8 Hz, 1 H) 2.87 (br d, J = 10.7 Hz, |
| | 1 H) 2.99-3.09 (m, 1 H) 3.13 (br d, J = 11.0 Hz, 1 H) 3.62-3.80 (m, 2 H) |
| | 7.15 (dd, J = 7.9, 4.8 Hz, 1 H) 7.26 (s, 1 H) 7.87 (br s, 1 H) 8.23 (br s, 1 H) 11.95 (br s, 1 |
| | H) 12.24-13.17 (m, 1 H). |
| 20 | ¹H NMR (500 MHz, CDCl₃) δ ppm 1.74-1.90 (m, 3 H) 2.05 (br d, J = 5.8 Hz, 1 H) |
| | 2.12-2.24 (m, 1 H) 2.30 (s, 3 H) 2.60 (br t, J = 11.0 Hz, 1 H) 3.04 (br d, J = 10.4 Hz, |
| | 1 H) 3.16 (br d, J = 10.4 Hz, 2 H) 3.68-3.82 (m, 5 H) 7.15 (dd, J = 7.9, 4.8 Hz, |
| | 1 H) 7.20 (s, 1 H) 7.58 (dd, J = 8.1, 1.4 Hz, 1 H) 8.48 (dd, J = 4.8, 1.6 Hz, 1 H) |
| | 11.66 (br s, 1 H). |
| 21 | ¹H NMR (400 MHz, DMSO-d₆) δ ppm 1.49 (qd, J = 12.3, 4.4 Hz, 1 H) |
| | 1.60-1.81 (m, 2 H) 1.96-2.09 (m, 2 H) 2.12 (s, 3 H) 2.31-2.40 (m, 1 H) 2.87-2.98 (m, 1 |
| | H) 3.06-3.15 (m, 1 H) 3.24 (tt, J = 11.1, 3.7 Hz, 1 H) 3.74 (s, 2 H) 3.79 (s, 3 H) |
| | 7.21 (dd, J = 8.0, 4.7 Hz, 1 H) 7.28 (s, 1 H) 7.95 (dd, J = 8.0, 1.5 Hz, 1 H) 8.28 (dd, |
| | J = 4.6, 1.4 Hz, 1 H); 1H exchanged. |
| 22 | ¹H NMR (400 MHz, CDCl₃) δ ppm 0.91 (d, J = 6.5 Hz, 3 H) 1.35-1.48 (m, 1 H) |
| | 1.65-1.76 (m, 1 H) 1.77-1.86 (m, 1 H) 2.27 (s, 5 H) 2.66 (td, J = 9.5, 3.6 Hz, 1 H) |
| | 2.88 (br d, J = 10.9 Hz, 1 H) 2.97 (br dd, J = 11.0, 2.7 Hz, 1 H) 3.59-3.75 (m, 2 H) |
| | 6.41 (s, 1 H) 7.01 (dd, J = 8.1, 4.6 Hz, 1 H) 7.14 (s, 1 H) 7.61 (d, J = 8.1 Hz, 1 H) |
| | 8.36 (dd, J = 4.9, 1.4 Hz, 1 H) 9.37 (br s, 1 H) 12.18 (br s, 1 H). |
| 23 | ¹H NMR (400 MHz, CDCl₃) δ ppm 0.86 (d, J = 6.7 Hz, 3 H) 1.38-1.57 (m, 2 H) |
| | 1.78-1.90 (m, 1 H) 2.24 (td, J = 11.3, 3.2 Hz, 1 H) 2.28 (s, 3 H) 2.50 (dd, J = 11.3, |
| | 3.0 Hz, 1 H) 3.09-3.13 (m, 1 H) 3.13-3.20 (m, 1 H) 3.24 (br dt, J = 11.1, 2.5 Hz, |
| | 1 H) 3.64-3.70 (m, 1 H) 3.78 (dd, J = 13.9, 0.7 Hz, 1 H) 6.39 (s, 1 H) 7.07 (dd, |
| | J = 8.1, 4.9 Hz, 1 H) 7.20 (s, 1 H) 7.83 (d, J = 8.1 Hz, 1 H) 8.37 (dd, J = 4.9, 1.4 Hz, 1 |
| | H) 10.49 (br s, 1 H) 11.07 (br s, 1 H). |
| 24 | ¹H NMR (400 MHz, CDCl₃) δ ppm 1.52-1.65 (m, 1 H) 2.06 (dtd, J = 12.7, 8.5, |
| | 8.5, 4.0 Hz, 1 H) 2.30 (s, 3 H) 2.50 (q, J = 8.1 Hz, 1 H) 2.55-2.73 (m, 3 H) |
| | 2.83 (td, J = 8.7, 4.0 Hz, 1 H) 2.94 (d, J = 6.2 Hz, 2 H) 3.73-3.84 (m, 2 H) 6.42 (s, 1 H) |
| | 7.03 (dd, J = 8.2, 4.7 Hz, 1 H) 7.21 (s, 1 H) 7.67 (d, J = 8.1 Hz, 1 H) 8.37 (dd, J = 4.7, |
| | 1.3 Hz, 1 H) 9.35 (br s, 1 H) 11.87 (br s, 1 H). |
| 25 | ¹H NMR (500 MHz, CDCl₃) δ ppm 1.53-1.66 (m, 1 H) 2.09-2.19 (m, 1 H) |
| | 2.29 (s, 3 H) 2.42 (dd, J = 9.0, 5.5 Hz, 1 H) 2.58-2.80 (m, 4 H) 2.86 (dd, J = 7.5, 4.0 Hz, |
| | 2 H) 3.68 (s, 3 H) 3.73-3.83 (m, 2 H) 6.42 (s, 1 H) 7.04 (dd, J = 8.2, 4.8 Hz, 1 H) |
| | 7.19 (s, 1 H) 7.52 (d, J = 8.1 Hz, 1 H) 8.37 (dd, J = 4.8, 1.3 Hz, 1H) 11.49 (br s, 1 |
| | H). |
| 26 | ¹H NMR (500 MHz, CDCl₃) δ ppm 1.04-1.13 (m, 1 H) 1.50-1.61 (m, 1 H) |
| | 1.65-1.82 (m, 2 H) 1.93-2.06 (m, 2 H) 2.07-2.17 (m, 1 H) 2.28 (s, 3 H) |
| | 2.64-2.86 (m, 4 H) 3.59-3.70 (m, 2 H) 3.69 (s, 3 H) 6.42 (s, 1 H) 7.04 (dd, J = 8.2, 4.8 Hz, 1 |
| | H) 7.17 (s, 1 H) 7.53 (d, J = 8.1 Hz, 1 H) 8.37 (dd, J = 4.8, 1.3 Hz, 1 H) 11.53 (br s, |
| | 1H). |
| 27 | ¹H NMR (400 MHz, CDCl₃) δ ppm 2.31 (s, 3 H) 2.38-2.48 (m, 2 H) 2.79 (br d, |
| | J = 10.4 Hz, 1 H) 3.10 (br d, J = 10.4 Hz, 1 H) 3.72-3.77 (m, 2 H) 3.80-3.88 (m, 1 |
| | H) 3.99 (dt, J = 11.3, 3.0 Hz, 1 H) 4.89 (dd, J = 9.0, 2.3 Hz, 1 H) 6.53 (s, 1 H) |
| | 7.08 (dd, J = 8.2, 4.7 Hz, 1 H) 7.23 (s, 1 H) 7.65 (dt, J = 8.1, 1.1 Hz, 1 H) 8.42 (dd, J = 4.9, |
| | 1.4 Hz, 1 H) 9.05 (br s, 1 H) 11.97 (br s, 1 H). |
| 29 | ¹H NMR (500 MHz, CDCl₃) δ ppm 1.39 (d, J = 6.9 Hz, 3 H) 1.59 (br dd, J = 9.1, 3.9 Hz, |
| | 1 H) 1.65-1.76 (m, 2 H) 1.80-1.90 (m, 1 H) 2.44 (br s, 1 H) 2.58-2.75 (m, |
| | 2 H) 2.83 (br s, 1 H) 3.10-3.26 (m, 1 H) 3.55 (q, J = 6.7 Hz, 1 H) 5.94 (d, J = 1.4 Hz, |
| | 1 H) 5.96 (d, J = 1.4 Hz, 1 H) 6.38 (s, 1 H) 6.73-6.80 (m, 2 H) 6.87 (s, 1 H) |
| | 7.01 (dd, J = 8.1, 4.6 Hz, 1 H) 7.57 (d, J = 8.1 Hz, 1 H) 8.37 (br d, J = 4.3 Hz, 1 H) |
| | 9.88 (br s, 1 H). |
| 30 | ¹H NMR (500 MHz, CDCl₃) δ ppm 1.41 (d, J = 6.6 Hz, 3 H) 1.53-1.63 (m, 1 H) |
| | 1.64-1.76 (m, 1 H) 1.66-1.73 (m, 1 H) 1.77-1.90 (m, 1 H) 2.29-2.45 (m, 1 H) |
| | 2.65 (br s, 1 H) 2.79 (br s, 2 H) 3.15-3.24 (m, 1 H) 3.49 (q, J = 6.6 Hz, 1 H) |
| | 5.93 (d, J = 1.4 Hz, 1 H) 5.95 (d, J = 1.4 Hz, 1 H) 6.39 (s, 1 H) 6.71-6.78 (m, 2 H) |
| | 6.85 (d, J = 1.4 Hz, 1 H) 7.02 (dd, J = 8.1, 4.6 Hz, 1 H) 7.60 (d, J = 8.1 Hz, 1 H) 8.38 (br d, |
| | J = 4.0 Hz, 1 H) 9.96 (br s, 1 H). |
| 31 | ¹H NMR (500 MHz, CDCl₃) δ ppm 1.41 (d, J = 6.6 Hz, 3 H) 1.51-1.92 (m, 4 H) |
| | 2.37 (br s, 1 H) 2.64 (br s, 1 H) 2.81 (br s, 2 H) 3.14-3.25 (m, 1 H) |
| | 3.43-3.55 (m, 1 H) 5.93 (d, J = 1.4 Hz, 1 H) 5.95 (d, J = 1.4 Hz, 1 H) 6.39 (s, 1 H) |
| | 6.63-6.79 (m, 2 H) 6.85 (d, J = 0.9 Hz, 1 H) 7.02 (dd, J = 8.1, 4.6 Hz, 1 H) 7.60 (d, J = 8.1 Hz, 1 |
| | H) 8.38 (d, J = 4.3 Hz, 1 H) 9.92 (br s, 1 H). |
| 32 | ¹H NMR (500 MHz, CDCl₃) δ ppm 1.40 (d, J = 6.6 Hz, 3 H) 1.55-1.98 (m, 4 H) |
| | 2.46 (br s, 1 H) 2.69 (br s, 2 H) 2.84 (br s, 1 H) 3.20 (br s, 1 H) 3.57 (q, J = 6.6 Hz, |
| | 1 H) 5.94 (d, J = 1.4 Hz, 1 H) 5.96 (d, J = 1.4 Hz, 1 H) 6.39 (s, 1 H) 6.71-6.81 (m, |
| | 2 H) 6.87 (s, 1 H) 7.01 (dd, J = 8.1, 4.6 Hz, 1 H) 7.58 (d, J = 8.1 Hz, 1 H) 8.37 (d, |
| | J = 4.0 Hz, 1 H) 9.88 (br s, 1 H). |
| 34 | ¹H NMR (500 MHz, CDCl₃) δ ppm 1.41 (d, J = 6.9 Hz, 3 H), 1.66-1.78 (m, 2 H), |
| | 1.80-1.92 (m, 1 H), 2.39-2.93 (m, 5 H), 3.19-3.28 (m, 1 H), 3.60 (q, J = 6.7 Hz, |
| | 1 H), 4.17-4.31 (m, 4 H), 6.37 (s, 1 H), 6.78-6.82 (m, 1 H), 6.82-6.85 (m, 1 H), |
| | 6.87 (d, J = 2.0 Hz, 1 H), 7.01 (dd, J = 8.1, 4.6 Hz, 1 H), 7.60 (d, J = 8.1 Hz, 1 H), |
| | 8.36 (dd, J = 4.8, 1.0 Hz, 1 H), 10.19 (br s, 1 H). |
| 35 | ¹H NMR (500 MHz, CDCl₃) δ ppm 1.42 (d, J = 6.6 Hz, 3 H), 1.56-1.77 (m, 2 H), |
| | 1.79-1.89 (m, 1 H), 2.40 (br s, 2 H), 2.70 (br s, 1 H), 2.80 (br s, 2 H), 3.23 (quin, |
| | J = 4.4 Hz, 1 H), 3.50 (q, J = 6.6 Hz, 1 H), 4.18-4.30 (m, 4 H), 6.38 (s, 1 H), |
| | 6.75-6.80 (m, 1 H), 6.80-6.83 (m, 1 H), 6.85 (d, J = 2.0 Hz, 1 H), 7.02 (dd, J = 8.1, 4.6 Hz, |
| | 1 H), 7.62 (d, J = 8.1 Hz, 1 H), 8.37 (dd, J = 4.8, 1.3 Hz, 1 H), 10.21 (br s, 1 H). |
| 36 | ¹H NMR (500 MHz, CDCl₃) δ ppm 1.44 (d, J = 6.6 Hz, 3 H), 1.58-1.77 (m, 2 H), |
| | 1.85 (br d, J = 8.4 Hz, 1 H), 2.46 (br s, 1 H), 2.77 (br s, 4 H), 3.23-3.30 (m, 1 H), |
| | 3.53 (q, J = 6.6 Hz, 1 H), 4.18-4.30 (m, 4 H), 6.38 (s, 1 H), 6.76-6.80 (m, 1 H), |
| | 6.80-6.84 (m, 1 H), 6.85 (d, J = 2.0 Hz, 1 H), 7.02 (dd, J = 8.1, 4.6 Hz, 1 H), |
| | 7.63 (d, J = 8.1 Hz, 1 H), 8.36 (dd, J = 4.6, 1.2 Hz, 1 H), 10.26 (br s, 1 H) |
| 37 | ¹H NMR (500 MHz, CDCl₃) δ ppm 1.42 (d, J = 6.9 Hz, 3 H), 1.61-1.77 (m, 2 H), |
| | 1.80-1.94 (m, 1 H), 2.38-2.96 (m, 5 H), 3.22-3.28 (m, 1 H), 3.62 (q, J = 6.7 Hz, |
| | 1 H), 4.19-4.30 (m, 4 H), 6.37 (s, 1 H), 6.79-6.82 (m, 1 H), 6.82-6.85 (m, 1 H), |
| | 6.87 (d, J = 1.7 Hz, 1 H), 7.01 (dd, J = 8.1, 4.6 Hz, 1 H), 7.60 (d, J = 8.1 Hz, 1 H), |
| | 8.36 (dd, J = 4.8, 1.3 Hz, 1H), 10.19 (br s, 1H) |
| 38 | ¹H NMR (300 MHz, CDCl₃) δ ppm 1.65-1.98 (m, 4 H) 2.38 (br s, 1 H) 2.62 (br |
| | d, J = 8.7 Hz, 1 H) 2.84 (br s, 2 H) 3.17-3.31 (m, 1 H) 3.36-3.59 (m, 2 H) |
| | 5.96 (d, J = 4.5 Hz, 2 H) 6.41 (s, 1 H) 6.78 (s, 2 H) 6.89 (s, 1 H) 7.03 (dd, J = 8.0, 4.7 Hz, |
| | 1 H) 7.59 (d, J = 8.1 Hz, 1 H) 8.38 (d, J = 4.7 Hz, 1 H) 9.90 (br s, 1 H). |
| 39 | ¹H NMR (300 MHz, CDCl₃) δ ppm 1.67-2.01 (m, 4 H) 2.62 (br s, 1 H) 2.89 (br s, |
| | 3 H) 3.44 (br s, 1 H) 3.54 (br d, J = 13.1 Hz, 1 H) 3.69 (br d, J = 12.5 Hz, 1 H) |
| | 4.26 (s, 4 H) 6.47 (s, 1 H) 6.87 (s, 2 H) 6.94 (s, 1 H) 7.09 (dd, J = 8.1, 4.9 Hz, 1 H) |
| | 7.70 (d, J = 8.1 Hz, 1 H) 8.38 (d, J = 4.0 Hz, 1 H) 10.34 (br s, 1 H). |
| 40 | ¹H NMR (500 MHz, CDCl₃) δ ppm 1.32-1.40 (m, 3 H) 1.43-1.56 (m, 1 H) |
| | 1.64-1.88 (m, 2H) 1.90-2.14 (m, 3 H) 2.88-3.00 (m, 1 H) 3.00-3.12 (m, 1.6 H) |
| | 3.15 (br d, J = 11.3 Hz, 0.4 H) 3.40 (q, J = 6.6 Hz, 0.6 H) 3.50 (q, J = 6.7 Hz, 0.4 H) |
| | 3.61 (s, 1.8 H) 3.69 (s, 1.2 H) 4.22-4.28 (m, 4 H) 6.41 (s, 0.6 H) 6.45 (s, 0.4 H) |
| | 6.75-6.86 (m, 3 H) 7.04 (td, J = 8.5, 4.6 Hz, 1 H) 7.50 (d, J = 8.1 Hz, 0.6 H) |
| | 7.53 (d, J = 8.1 Hz, 0.4 H) 8.37 (dd, J = 4.6, 1.4 Hz, 0.6 H) 8.39 (dd, J = 4.8, 1.3 Hz, 0.4 |
| | H). |
| 41 | ¹H NMR (400 MHz, CDCl₃) δ ppm 1.41-1.54 (m, 1 H) 1.65-1.82 (m, 2 H) |
| | 1.98-2.08 (m, 1 H) 2.11-2.26 (m, 2 H) 2.91 (br d, J = 11.1 Hz, 1 H) 3.05-3.21 (m, 2 |
| | H) 3.68 (s, 3 H) 3.83 (s, 2 H) 6.38 (s, 1 H) 7.04 (dd, J = 8.3, 4.6 Hz, 1 H) 7.77 (dt, |
| | J = 8.1, 1.1 Hz, 1 H) 7.90 (dd, J = 8.6, 1.8 Hz, 1 H) 8.03 (d, J = 1.2 Hz, 1 H) 8.08 (d, |
| | J = 8.8 Hz, 1 H) 8.23 (dd, J = 4.6, 1.4 Hz, 1 H) 8.89-8.95 (m, 2 H). |
| 42 | ¹H NMR (400 MHz, CDCl₃) δ ppm 1.48-1.67 (m, 1 H) 1.73-1.91 (m, 2 H) |
| | 2.06-2.16 (m, 1 H) 2.17-2.30 (m, 2 H) 3.01 (br d, J = 11.1 Hz, 1 H) 3.06-3.20 (m, 2 |
| | H) 3.66 (s, 3 H) 3.74-3.92 (m, 2 H) 6.48 (s, 1 H) 7.04 (dd, J = 8.2, 4.7 Hz, 1 H) |
| | 7.50 (d, J = 8.1 Hz, 1 H) 7.87 (dd, J = 8.6, 1.8 Hz, 1 H) 8.06 (d, J = 1.2 Hz, 1 H) |
| | 8.09 (d, J = 8.8 Hz, 1 H) 8.38 (dd, J = 4.6, 1.4 Hz, 1 H) 8.77-8.87 (m, 2 H) |
| 43 | ¹H NMR (400 MHz, CDCl₃) δ ppm 1.51-1.64 (m, 1 H) 1.75-1.90 (m, 2 H) |
| | 2.06-2.14 (m, 1 H) 2.16-2.30 (m, 2 H) 3.01 (br d, J = 11.1 Hz, 1 H) 3.06-3.17 (m, 2 |
| | H) 3.66 (s, 3 H) 3.77-3.88 (m, 2 H) 6.48 (s, 1 H) 7.04 (dd, J = 8.2, 4.7 Hz, 1 H) |
| | 7.50 (br d, J = 8.3 Hz, 1 H) 7.87 (dd, J = 8.7, 1.7 Hz, 1 H) 8.06 (d, J = 1.2 Hz, 1 H) |
| | 8.09 (d, J = 8.6 Hz, 1 H) 8.38 (dd, J = 4.7, 1.3 Hz, 1 H) 8.79-8.88 (m, 2 H). |
| 45 | ¹H NMR (500 MHz, CDCl₃) δ ppm 1.36 (d, J = 6.9 Hz, 3 H) 1.48 (qd, J = 12.4, 4.3 Hz, |
| | 1 H) 1.68 (qt, J = 12.6, 3.8 Hz, 1 H) 1.74-1.81 (m, 1 H) 1.97-2.06 (m, 2 H) |
| | 2.10 (t, J = 10.8 Hz, 1 H) 2.90 (br d, J = 11.3 Hz, 1 H) 3.04 (tt, J = 11.1, 3.3 Hz, 1 H) |
| | 3.16 (dt, J = 11.1, 1.7 Hz, 1 H) 3.44-3.53 (m, 1 H) 3.69 (s, 3 H) 5.91-5.97 (m, 2 |
| | H) 6.45 (s, 1 H) 6.75 (d, J = 0.9 Hz, 2 H) 6.88 (s, 1 H) 7.04 (dd, J = 8.4, 4.6 Hz, 1 H) |
| | 7.52 (d, J = 8.1 Hz, 1 H) 8.38 (dd, J = 4.6, 1.4 Hz, 1 H). |
| 47 | ¹H NMR (500 MHz, CDCl₃) δ ppm 1.37 (d, J = 6.6 Hz, 3 H) 1.50 (qd, J = 12.3, 4.3 Hz, |
| | 1 H) 1.72-1.90 (m, 2H) 1.96 (t, J = 10.8 Hz, 1 H) 2.00-2.11 (m, 2 H) |
| | 2.95 (tt, J = 11.0, 3.5 Hz, 1 H) 3.01 (br dd, J = 11.3, 1.4 Hz, 1 H) 3.10 (br d, J = 10.7 Hz, 1 |
| | H) 3.40 (q, J = 6.6 Hz, 1 H) 3.60 (s, 3 H) 5.90-5.94 (m, 2 H) 6.42 (s, 1 H) |
| | 6.70-6.77 (m, 2 H) 6.86 (d, J = 0.9 Hz, 1 H) 7.02 (dd, J = 8.2, 4.8 Hz, 1 H) 7.49 (d, J = 8.1 Hz, |
| | 1 H) 8.36 (dd, J = 4.6, 1.2 Hz, 1 H). |
| 46 | ¹H NMR (400 MHz, CDCl₃) δ ppm 1.37 (d, J = 6.7 Hz, 3 H) 1.50 (qd, J = 12.3, 4.4 Hz, |
| | 1 H) 1.72-1.90 (m, 2 H) 1.95 (t, J = 10.8 Hz, 1 H) 2.00-2.11 (m, 2 H) |
| | 2.90-2.99 (m, 1 H) 2.99-3.05 (m, 1 H) 3.10 (br d, J = 10.9 Hz, 1 H) 3.39 (q, J = 6.7 Hz, 1 |
| | H) 3.60 (s, 3 H) 5.91-5.95 (m, 2 H) 6.41 (s, 1 H) 6.70-6.77 (m, 2 H) 6.86 (s, 1 |
| | H) 7.03 (dd, J = 8.2, 4.7 Hz, 1 H) 7.50 (dt, J = 8.1, 1.0 Hz, 1 H) 8.37 (dd, J = 4.7, 1.3 Hz, |
| | 1 H). |
| 48 | ¹H NMR (500 MHz, CDCl₃) δ ppm 1.36 (d, J = 6.9 Hz, 3 H) 1.48 (qd, J = 12.4, 4.0 Hz, |
| | 1 H) 1.69 (qt, J = 12.5, 3.8 Hz, 1 H) 1.74-1.82 (m, 1 H) 1.98-2.06 (m, 2 H) |
| | 2.10 (t, J = 10.8 Hz, 1 H) 2.90 (br d, J = 11.3 Hz, 1 H) 3.00-3.09 (m, 1 H) 3.16 (dt, |
| | J = 11.1, 1.7 Hz, 1 H) 3.41-3.89 (m, 3 H) 3.50 (q, J = 6.7 Hz, 1 H) 5.89-6.00 (m, 2 |
| | H) 6.45 (s, 1 H) 6.75 (d, J = 0.9 Hz, 2 H) 6.88 (s, 1 H) 7.04 (dd, J = 8.1, 4.6 Hz, 1 H) |
| | 7.52 (d, J = 8.1 Hz, 1 H) 8.38 (dd, J = 4.8, 1.3 Hz, 1 H). |
| 49 | ¹H NMR (300 MHz, DMSO-d₆) δ ppm 1.63-1.97 (m, 7 H), 2.12 (br d, J = 10.6 Hz, |
| | 1 H), 2.93 (br s, 1 H), 3.35 (br s, 8 H), 3.99 (br s, 2 H), 6.46 (s, 1 H), 7.17 (dd, |
| | J = 8.0, 5.1 Hz, 1 H), 7.86 (br d, J = 8.1 Hz, 1 H), 8.33 (d, J = 4.4 Hz, 1 H), 11.90 (br |
| | s, 1 H); 1H exchanged. |
| 50 | ¹H NMR (400 MHz, CDCl₃) δ ppm 1.46-1.60 (m, 1 H), 1.82 (dt, J = 6.6, 3.3 Hz, 2 |
| | H), 2.01-2.25 (m, 3 H), 2.98 (br d, J = 11.1 Hz, 1 H), 3.07-3.19 (m, 2 H), 3.60 (d, |
| | J = 0.9 Hz, 2 H), 3.69 (s, 3 H), 4.25-4.29 (m, 2 H), 4.30-4.35 (m, 2 H), 6.46 (s, 1 |
| | H), 6.90 (s, 1 H), 7.04 (dd, J = 8.2, 4.7 Hz, 1 H), 7.48-7.56 (m, 1 H), 8.13 (s, 1 H), |
| | 8.38 (dd, J = 4.9, 1.4 Hz, 1H) |
| 51 | ¹H NMR (400 MHz, CDCl₃) δ ppm 1.52-1.65 (m, 1 H), 1.80-1.88 (m, 2 H), |
| | 2.05-2.13 (m, 1 H), 2.22-2.37 (m, 2 H), 2.96-3.05 (m, 1 H), 3.10-3.19 (m, 2 |
| | H), 3.66 (s, 3 H), 3.86-3.97 (m, 2 H), 6.50 (s, 1 H), 7.03 (dd, J = 8.2, 4.7 Hz, 1 H), |
| | 7.47-7.54 (m, 2 H), 7.65 (d, J = 8.6 Hz, 1 H), 7.69 (ddd, J = 8.4, 6.9, 1.5 Hz, 1 H), |
| | 7.80 (dd, J = 8.2, 1.0 Hz, 1 H), 8.08 (d, J = 8.8 Hz, 1 H), 8.13 (d, J = 8.3 Hz, 1 H), |
| | 8.37 (dd, J = 4.6, 1.2 Hz, 1 H). |
| 52 | ¹H NMR (500 MHz, CDCl₃) δ ppm 1.56 (qd, J = 12.1, 4.6 Hz, 1 H), 1.74-1.89 (m, |
| | 2 H), 2.06-2.12 (m, 1 H), 2.15-2.29 (m, 2 H), 2.98 (br d, J = 11.0 Hz, 1 H), |
| | 3.05-3.14 (m, 2 H), 3.65 (s, 3 H), 3.68-3.73 (m, 1 H), 3.73-3.78 (m, 1 H), 6.48 (s, 1 |
| | H), 7.03 (dd, J = 8.1, 4.6 Hz, 1 H), 7.49 (d, J = 8.4 Hz, 1 H), 7.69 (dd, J = 9.0, 1.4 Hz, |
| | 1 H), 7.92 (s, 1 H), 7.95 (d, J = 9.0 Hz, 1 H), 8.37 (dd, J = 4.6, 1.2 Hz, 1 H). |
| 53 | ¹H NMR (400 MHz, CDCl₃) δ ppm 1.47-1.61 (m, 1 H), 1.72-1.87 (m, 2 H), |
| | 2.03-2.24 (m, 3 H), 2.99 (br d, J = 11.3 Hz, 1 H), 3.03-3.17 (m, 2 H), |
| | 3.62-3.68 (m, 3 H), 3.69-3.80 (m, 2 H), 6.47 (s, 1 H), 7.03 (dd, J = 8.1, 4.6 Hz, 1 H), |
| | 7.46-7.52 (m, 2 H), 7.91 (d, J = 8.3 Hz, 1 H), 8.12 (d, J = 0.9 Hz, 1 H), 8.37 (dd, J = 4.6, |
| | 1.4 Hz, 1 H), 8.99 (s, 1 H). |
| 54 | ¹H NMR (400 MHz, CDCl₃) δ ppm 1.45-1.58 (m, 1 H) 1.63-1.76 (m, 1 H) |
| | 1.76-1.85 (m, 1 H) 2.00-2.08 (m, 1 H) 2.12 (td, J = 11.4, 2.8 Hz, 1 H) 2.19 (t, J = 10.8 Hz, |
| | 1 H) 2.92-3.03 (m, 2 H) 3.06-3.14 (m, 1 H) 3.59 (s, 3 H) 3.62-3.74 (m, 2 |
| | H) 3.81 (s, 3 H) 6.37 (s, 1 H) 6.47 (s, 1 H) 7.02 (dd, J = 8.1, 4.6 Hz, 1 H) 7.06 (td, |
| | J = 7.5, 1.0 Hz, 1 H) 7.18 (ddd, J = 8.2, 7.1, 1.2 Hz, 1 H) 7.30 (dd, J = 8.1, 0.7 Hz, 1 |
| | H) 7.47 (d, J = 8.1 Hz, 1 H) 7.54 (d, J = 7.6 Hz, 1 H) 8.37 (dd, J = 4.6, 1.4 Hz, 1 H). |
| 55 | ¹H NMR (400 MHz, CDCl₃) δ ppm 1.45-1.59 (m, 1 H), 1.68-1.87 (m, 2 H), |
| | 2.00-2.19 (m, 3 H), 2.93 (br d, J = 10.9 Hz, 1 H), 3.00-3.12 (m, 2 H), |
| | 3.44-3.55 (m, 2 H), 3.68 (s, 3 H), 4.21-4.28 (m, 2 H), 4.41 (dt, J = 3.9, 2.1 Hz, 2 H), 6.46 (s, |
| | 1 H), 7.05 (dd, J = 8.2, 4.7 Hz, 1 H), 7.20 (d, J = 2.1 Hz, 1 H), 7.53 (dt, J = 8.1, 1.0 Hz, |
| | 1 H), 7.76 (d, J = 2.1 Hz, 1 H), 8.38 (dd, J = 4.6, 1.4 Hz, 1 H). |
| 56 | ¹H NMR (500 MHz, CDCl₃) δ ppm 1.56 (qd, J = 12.1, 4.0 Hz, 1 H) 1.68-1.79 (m, |
| | 1 H) 1.79-1.87 (m, 1 H) 2.02-2.10 (m, 1 H) 2.30 (td, J = 11.3, 2.9 Hz, 1 H) |
| | 2.39 (t, J = 10.5 Hz, 1 H) 2.93 (br d, J = 11.3 Hz, 1 H) 3.06 (tt, J = 10.6, 3.7 Hz, 1 H) |
| | 3.12 (dt, J = 11.2, 1.6 Hz, 1 H) 3.63 (s, 3 H) 3.88 (s, 2 H) 3.89 (s, 3 H) 6.49 (s, 1 H) |
| | 7.03 (dd, J = 8.4, 4.6 Hz, 1 H) 7.22-7.30 (m, 2 H) 7.31-7.38 (m, 1 H) 7.41-7.59 (m, 1 |
| | H) 7.73 (dt, J = 7.8, 1.0 Hz, 1 H) 8.38 (dd, J = 4.6, 1.4 Hz, 1 H). |
| 57 | ¹H NMR (500 MHz, CDCl₃) δ ppm 1.98 (br s, 1 H), 2.29 (s, 3 H), 2.40-2.59 (m, |
| | 2 H), 2.76 (br d, J = 10.4 Hz, 1 H), 2.94-3.03 (m, 2 H), 3.05-3.12 (m, 1 H), |
| | 3.68-3.78 (m, 2 H), 4.23 (dd, J = 7.8, 3.2 Hz, 1 H), 6.52 (s, 1 H), 7.03-7.08 (m, 1 H), |
| | 7.21 (s, 1 H), 7.63 (d, J = 8.1 Hz, 1 H), 8.40 (dd, J = 4.6, 1.4 Hz, 1 H), 9.58 (br s, 1 |
| | H), 12.12 (br s, 1 H). |
| 58 | ¹H NMR (500 MHz, CDCl₃) δ ppm 1.55-1.65 (m, 1 H), 2.04-2.21 (m, 1 H), |
| | 2.29 (s, 3 H), 2.42 (dd, J = 9.2, 5.5 Hz, 1 H), 2.57-3.00 (m, 6 H), 3.68 (s, 3 H), |
| | 3.72-3.88 (m, 2 H), 6.43 (s, 1 H), 7.04 (dd, J = 8.2, 4.8 Hz, 1 H), 7.19 (s, 1 H), |
| | 7.52 (d, J = 8.1 Hz, 1 H), 8.38 (dd, J = 4.8, 1.3 Hz, 1 H), 11.67 (br s, 1 H). |
| 59 | ¹H NMR (400 MHz, CDCl₃) δ ppm 1.54-1.65 (m, 1 H), 2.10-2.22 (m, 1 H), |
| | 2.30 (s, 3 H), 2.42 (dd, J = 9.0, 5.3 Hz, 1 H), 2.55-2.94 (m, 6 H), 3.68 (s, 3 H), |
| | 3.72-3.86 (m, 2 H), 6.42 (s, 1 H), 7.04 (dd, J = 8.1, 4.6 Hz, 1 H), 7.19 (s, 1 H), |
| | 7.52 (d, J = 8.1 Hz, 1 H), 8.38 (dd, J = 4.9, 1.4 Hz, 1 H), 11.65 (br s, 1 H). |
| 60 | ¹H NMR (500 MHz, CDCl₃) δ ppm 1.54-1.65 (m, 1 H), 2.08-2.18 (m, 1 H), |
| | 2.29 (s, 3 H), 2.41 (dd, J = 9.2, 5.8 Hz, 1 H), 2.61-2.74 (m, 3 H), 2.77-2.90 (m, 3 |
| | H), 3.74-3.78 (m, 1 H), 3.78 (s, 3 H), 3.79-3.83 (m, 1 H), 6.18 (s, 1 H), |
| | 7.00 (dd, J = 7.8, 4.6 Hz, 1 H), 7.20 (s, 1 H), 7.78 (dd, J = 7.7, 1.6 Hz, 1 H), 8.24 (dd, |
| | J = 4.6, 1.4 Hz, 1 H), 11.42 (br s, 1 H). |
| 61 | ¹H NMR (500 MHz, CDCl₃) δ ppm 1.55-1.64 (m, 1 H), 2.09-2.19 (m, 1 H), |
| | 2.29 (s, 3 H), 2.41 (dd, J = 9.2, 5.5 Hz, 1 H), 2.58-2.69 (m, 2 H), 2.70-2.78 (m, 2 |
| | H), 2.79-2.91 (m, 2H), 3.76 (s, 3 H), 3.73-3.78 (m, 1 H), 3.79-3.83 (m, 1 H), |
| | 6.24 (s, 1 H), 7.20 (s, 1 H), 7.40 (dd, J = 5.5, 0.9 Hz, 1 H), 8.19 (d, J = 5.5 Hz, 1 H), |
| | 8.67 (s, 1 H), 11.74 (br s, 1 H). |
| 62 | ¹H NMR (400 MHz, CDCl₃) δ ppm 1.57-1.67 (m, 1 H), 1.98-2.09 (m, 1 H), |
| | 2.30 (s, 3 H), 2.36 (dd, J = 8.7, 5.2 Hz, 1 H), 2.52-2.68 (m, 3 H), 2.75 (td, J = 9.0, |
| | 5.3 Hz, 1 H), 3.08 (d, J = 7.4 Hz, 2 H), 3.71-3.85 (m, 5 H), 7.18 (dd, J = 8.3, 4.6 Hz, |
| | 1 H), 7.19 (s, 1 H), 7.60 (dd, J = 8.3, 1.2 Hz, 1 H), 8.56 (dd, J = 4.6, 1.4 Hz, 1 |
| | H), 11.83 (br s, 1 H). |
| 63 | ¹H NMR (500 MHz, CDCl₃) δ ppm 1.57 (qd, J = 12.2, 4.6 Hz, 1 H), 1.75-1.89 (m, |
| | 2 H), 2.05-2.12 (m, 1 H), 2.13-2.22 (m, 2 H), 3.01 (br d, J = 11.3 Hz, 1 H), |
| | 3.04-3.13 (m, 2 H), 3.62 (s, 3 H), 3.70 (d, J = 13.3 Hz, 1 H), 3.79 (d, J = 13.3 Hz, 1 H), |
| | 6.48 (s, 1 H), 7.03 (dd, J = 8.1, 4.6 Hz, 1 H), 7.39 (dd, J = 8.2, 4.2 Hz, 1 H), |
| | 7.47-7.51 (m, 1 H), 7.73 (d, J = 1.2 Hz, 1 H), 7.79 (dd, J = 8.7, 2.0 Hz, 1 H), 8.07 (d, |
| | J = 8.7 Hz, 1 H), 8.12 (dd, J = 8.4, 0.9 Hz, 1 H), 8.37 (dd, J = 4.8, 1.3 Hz, 1 H), |
| | 8.88 (dd, J = 4.2, 1.6 Hz, 1 H). |
| 64 | ¹H NMR (500 MHz, CDCl₃) δ ppm 1.08-1.19 (m, 1 H), 1.47-1.76 (m, 3 H), |
| | 1.98 (br t, J = 9.2 Hz, 1 H), 2.15-2.26 (m, 2 H), 2.30 (s, 3 H), 2.48-2.67 (m, 2 H), |
| | 3.53-3.66 (m, 2 H), 4.00 (dd, J = 14.4, 6.9 Hz, 1 H), 4.13 (dd, J = 14.2, 8.4 Hz, 1 |
| | H), 6.68 (dd, J = 3.3, 0.7 Hz, 1 H), 7.11 (dd, J = 8.4, 4.6 Hz, 1 H), 7.14 (s, 1 H), |
| | 7.28 (d, J = 3.2 Hz, 1 H), 7.65 (d, J = 8.4 Hz, 1 H), 8.44 (dd, J = 4.6, 1.2 Hz, 1 H), |
| | 11.36 (br s, 1 H). |

### D. Pharmacological Examples

### 1) OGA—Biochemical Assay

The assay is based on the inhibition of the hydrolysis of fluorescein mono-B-D-N-Acetyl-Glucosamine (FM-GlcNAc) (Mariappa et al. 2015, Biochem J 470:255) by the recombinant human Meningioma Expressed Antigen 5 (MGEA5), also referred to as 0-GlcNAcase (OGA). The hydrolysis FM-GlcNAc (Marker Gene technologies, cat # M1485) results in the formation of β-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OriGene (cat # TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2 H₂O (Sigma, # C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 10 nM (protocol A) or 2 nM (protocol B) concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 1 h (10 nM OGA, protocol A) or 6 h (2 nM OGA, protocol B). To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

### 2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting O-GlcNAcylated residues as previously described (Dorfmueller et al. 2010 Chemistry & biology, 17:1250).

Inhibition of OGA will result in an increase of 0-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, # A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, # A-21042) and nuclei stained with Hoechst 33342 at a final concentration of 1 μg/ml in ICC (Lifetechnologies, # H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 14
Results in the biochemical and cellular assays.

| | | | | | Cellular |
|---|---|---|---|---|---|
| Co. | Enzymatic | Enzymatic | Enzymatic | Cellular | Eₘₐₓ |
| no. | protocol | hOGA; pIC₅₀ | Eₘₐₓ (%) | hOGA; pEC₅₀ | (%) |
| 1 | A | 5.73 | 75.9 | | |
| 2 | A | 6.58 | 101.8 | | |
| 3 | A | 6.25 | 99 | | |
| 4 | B | 6.73 | 99.4 | <5 | 44.3 |
| | A | 6.75 | 99.7 | | |
| 5 | A | 7.59 | 99.7 | | |
| 6 | B | 5.57 | 79.1 | 5.44 | 66.6 |
| | A | 5.78 | 92.3 | | |
| 7 | B | 7.60 | 100 | 7.67 | 113.7 |
| | A | 7.74 | 103.8 | | |
| 8 | B | 6.98 | 100 | 6.83 | 113.3 |
| | A | 6.95 | 101.1 | | |
| 9 | A | 6.69 | 98.0 | | |
| 10 | B | 5.99 | 90.1 | | |
| 11 | B | 7.13 | 100 | 7.74 | 103 |
| 12 | B | 7.37 | 123.6 | 7.75 | 120.4 |
| 13 | B | 7.64 | 98.9 | | |
| 14 | B | 6.17 | 96.3 | | |
| 15 | B | 8.09 | 101.2 | 7.58 | 123.8 |
| | A | 7.95 | 101.1 | | |
| 16 | B | 8.15 | 99.7 | 8.06 | 90.5 |
| 17 | B | 6.02 | 92.8 | 5.14 | 61.9 |
| 18 | A | 4.61 | 55.6 | | |
| 19 | A | 6.83 | 102 | 6.07 | 109.3 |
| 20 | A | 6.81 | 98.0 | 5.58 | 95.6 |
| 21 | A | 6.52 | 100.8 | 5.58 | 101.3 |
| 22 | B | 7.65 | 101.6 | 7.68 | 114 |
| | A | 7.38 | 101.1 | | |
| 23 | B | 7.03 | 102.13 | 6.62 | 111.9 |
| | A | 6.65 | 101.0 | | |
| 24 | B | 8.67 | 99.8 | 8.1 | 98.0 |
| 25 | B | 8.82 | 101.9 | 8.03 | 120.6 |
| 26 | B | 8.62 | 101 | >8.34 | 118.1 |
| 27 | B | 5.50 | 77.3 | | |
| 29 | B | 5.43 | 73.5 | | |
| 30 | B | 5.39 | 75.1 | | |
| 31 | B | 7.57 | 100.4 | 6.71 | 95.8 |
| 32 | B | 5.75 | 89.4 | | |
| 33^{&} | B | 7.26 | 98.6 | 6.38 | 106.7 |
| 34 | B | 6.15 | 93.8 | | |
| 35 | B | 5.61 | 87.7 | | |
| 36 | B | 7.96 | 101.3 | | |
| 37 | B | 6.02 | 96.0 | | |
| 38 | A | 5.76 | 99.9 | | |
| 39^{&} | A | 6.21 | 98.2 | 5.44 | 90.3 |
| 40 | B | 7.21 | 101.9 | 6.02 | 75.1 |
| 41 | B | 5.90 | 89.8 | | |
| 42 | B | 6.11 | 93.2 | | |
| 43 | B | <5 | 46.5 | | |
| 44 | B | 7.08 | 99.0 | | |
| 45 | B | 7.56 | 100 | | |
| 46 | B | <5 | 24.2 | | |
| 47 | B | 5.81 | 85.8 | | |
| 48 | B | <5 | 31.7 | | |
| 49 | B | 5.58 | 80.5 | <5 | 7.2 |
| 50 | B | 5.06 | 52.3 | | |
| 51 | B | 5.11 | 56.7 | | |
| 52 | B | 5.4 | 72.2 | | |
| 53 | B | 6.08 | 97.2 | | |
| 54 | B | 5.39 | 69.1 | | |
| 55 | B | 5.66 | 81.5 | | |
| 56 | B | 6.08 | 98 | | |
| 57 | B | 5.18 | 60.8 | | |
| 58 | B | 8.57 | 101.6 | | |
| 59 | B | 8.93 | 99.7 | | |
| 60 | B | 8.03 | 99.2 | | |
| 61 | B | 8.82 | 100 | | |
| 62 | B | | | | |
| 63 | B | <5 | 23.9 | | |
| 64 | B | 8.11 | 100.4 | | |

^{&}means•HCl

### Additional and Comparative Data

### PDE2 Inhibitory Activity

Compound 2 according to the invention was tested for PDE2 inhibitory activity (protocol as described in WO2017/076900, page 28) and was found to have no appreciable PDE2 inhibitory activity, displaying 33.11% effect at a screening concentration of 10 μM.

OGA Inhibitory Activity The OGA inhibitory activity of compounds 5 and 7 was compared with the corresponding 1,4-disubstituted piperidine compound (comparator) and found to be ˜1 order of magnitude greater.

| Co. No. | Structure | pIC₅₀ | Eₘₐₓ |
|---|---|---|---|
| Comparator | 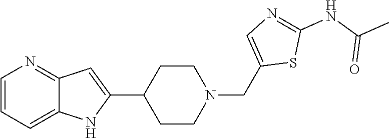 [SMILES: CC(=O)Nc1ncc(CN2CCC(c3cc4ncccc4[nH]3)CC2)s1] | 6.65 | 102.72 |
| 5 | 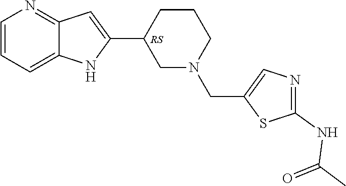 [SMILES: CC(=O)Nc1ncc(CN2CCCC(c3cc4ncccc4[nH]3)C2)s1] | 7.59 | 99.66 |
| | 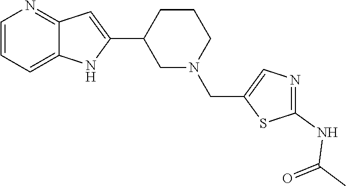 [SMILES: CC(=O)Nc1ncc(CN2CCCC(c3cc4ncccc4[nH]3)C2)s1] | | |
| 7 | 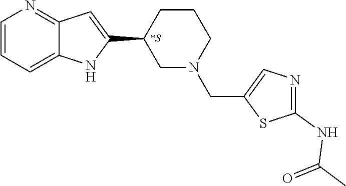 [SMILES: CC(=O)Nc1ncc(CN2CCC[C@H](c3cc4ncccc4[nH]3)C2)s1] | 7.74 | 103.79 |

1. A compound of Formula (I) 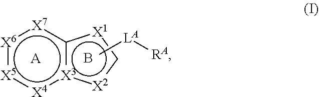
or a tautomer or a stereoisomeric form thereof, wherein
A-B represent a 9-membered bicyclic heteroaryl system having from 1 to 4 nitrogen atoms, wherein
X¹ and X² are each independently selected from the group consisting of C, CR^{x}, N, and NR^{y}; and
X³ is C or N;
X⁴, X⁵, X⁶, and X⁷ are each independently selected from the group consisting of CR^{x} and N;
with the proviso that at least one of X² and X³ is N or in the case of X², is N or NR^{y};
wherein each R^{x}, when present, is independently selected from the group consisting of hydrogen;
halo; —CN; C₁₋₄alkyl optionally substituted with 1, 2 or 3 independently selected halo substituents; and C₁₋₄alkyloxy optionally substituted with 1, 2 or 3 independently selected halo substituents;
each R^{y}, when present, is independently selected from the group consisting of hydrogen and C₁₋₄alkyl optionally substituted with 1, 2 or 3 independently selected halo substituents;
L^{A} is bound to any available carbon or nitrogen atom at the 5-membered B ring of the A-B bicycle, and is selected from a bond and CHR¹; wherein
R¹ is selected from the group consisting of hydrogen and C₁₋₄alkyl optionally substituted with 1, 2 or 3 independently selected halo substituents;
R^{A} is a radical selected from the group consisting of (a-1), (a-2) and (a-3) 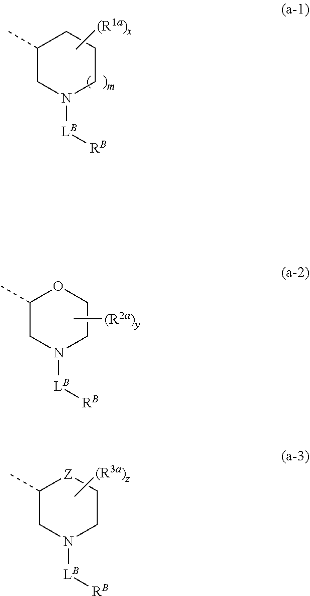
wherein
m represents 0 or 1;
x, y and z, each independently represent 0, 1 or 2;
each R^{1a} and R^{2a} when present, is bound to any available carbon atom and is independently selected from the group consisting of halo and C₁₋₄alkyl optionally substituted with 1, 2, or 3 independently selected halo substituents; or two R^{1a}, or two R^{2a} substituents are bound to the same carbon atom and together form a cyclopropylidene radical;
Z is N when substituted with R^{3a}, or NH;
each R^{3a} is bound to any available carbon or nitrogen atom when present and is independently selected from C₁₋₃alkyl optionally substituted with 1, 2 or 3 independently selected halo substituents; or two R^{1a} are bound to the same carbon atom and together form a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} is a radical selected from the group consisting of (b-1), (b-2), (b-3), (b-4), (b-5), (b-6), (b-7), (b-8), (b-9), (b-10), and (b-11): 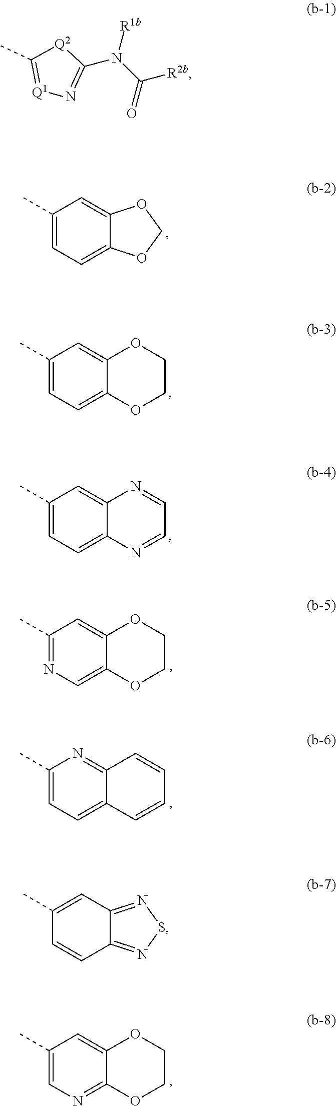 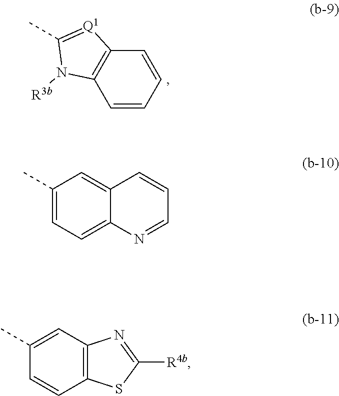
wherein
Q¹ is CH or N;
Q² is O, NR⁴a or S;
R^{4a}, R^{1b}, R^{3b} and R^{4b} are each independently selected from H and C₁₋₄alkyl; and
R^{2b} is C₁₋₄alkyl;
or -L^{B}-R^{B} is (b-12) 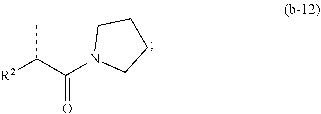
or a pharmaceutically acceptable addition salt or a solvate thereof.

2. The compound according to claim 1, wherein
X¹ is selected from the group consisting of CR^{x}, N, and NR^{y};
X² is N or NR^{y};
X³ is C or N;
X⁴, X⁵, X⁶, and X⁷ are each independently selected from the group consisting of CR^{x} and N;
wherein each R^{x}, when present, is independently selected from the group consisting of hydrogen;
halo; —CN; C₁₋₄alkyl optionally substituted with 1, 2 or 3 independently selected halo substituents; and C₁₋₄alkyloxy optionally substituted with 1, 2 or 3 independently selected halo substituents;
each R^{y}, when present, is independently selected from the group consisting of hydrogen and C₁₋₄alkyl optionally substituted with 1, 2 or 3 independently selected halo substituents;
L^{A} is bound to any available carbon or nitrogen atom at the 5-membered B ring of the A-B bicycle, and is selected from a bond and CHR¹; wherein
R¹ is selected from the group consisting of hydrogen and C₁₋₄alkyl optionally substituted with 1, 2 or 3 independently selected halo substituents;
R^{A} is a radical selected from the group consisting of (a-1), (a-2) and (a-3), wherein m represents 0 or 1;
x, y and z, each independently represent 0 or 1;
each R^{1a} and R^{2a} when present, is bound to any available carbon atom and is independently selected from the group consisting of halo and C₁₋₄alkyl optionally substituted with 1, 2, or 3 independently selected halo substituents; or two R^{1a}, or two R^{e}a substituents are bound to the same carbon atom and together form a cyclopropylidene radical;
Z is N when substituted with R^{3a}, or NH;
each R^{3a} is bound to any available carbon or nitrogen atom when present and is independently selected from C₁₋₃alkyl optionally substituted with 1, 2 or 3 independently selected halo substituents; or two R^{3a} are bound to the same carbon atom and together form a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} is a radical selected from the group consisting of (b-1), (b-2), (b-3), (b-4), (b-5), (b-6), (b-7), (b-8), (b-9), (b-10), and (b-11) or -L^{B}-R^{B} is (b-12).

3. The compound according to claim 1, wherein
X¹ is selected from the group consisting of CR^{x}, N, and NR^{y};
X² is N or NR^{y};
X³ is C;
X⁴, X⁵, X⁶, and X⁷ are each independently selected from the group consisting of CR^{x} and N;
wherein each R^{x}, when present, is independently selected from the group consisting of hydrogen;
halo; C₁₋₄alkyl optionally substituted with 1, 2 or 3 independently selected halo substituents; and
C₁₋₄alkyloxy optionally substituted with 1, 2 or 3 independently selected halo substituents;
each R^{y}, when present, is independently selected from the group consisting of hydrogen and C₁₋₄alkyl;
L^{A} is bound to any available carbon or nitrogen atom at the 5-membered B ring of the A-B bicycle, and is selected from a bond and CH₂;
R^{A} is a radical selected from the group consisting of (a-1), (a-2) and (a-3), wherein m represents 0 or 1;
x, y and z, each independently represent 0 or 1;
each R^{1a} and R^{2a} when present, is C₁₋₄alkyl bound to any available carbon atom;
Z is NH;
each R^{1a} when present, is C₁₋₃alkyl bound to any available carbon;
L^{B} is selected from the group consisting of >CHR² and >SO₂;
wherein R² is selected from the group consisting of hydrogen and C₁₋₄alkyl; and
R^{B} is a radical selected from the group consisting of (b-1), (b-2), (b-3), (b-4), (b-5), (b-6), (b-7), (b-8), (b-9), (b-10), and (b-11), wherein Q¹ is CH or N; Q² is S; R^{4a}, R^{1b}, R^{3b} and R^{4b} are each independently selected from H and CH₃;
and R^{2b} is C₁₋₄alkyl;
or -L^{B}-R^{B} is (b-12′)

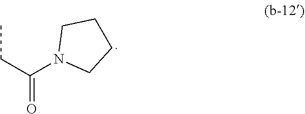

4. The compound according to claim 1, having the Formula 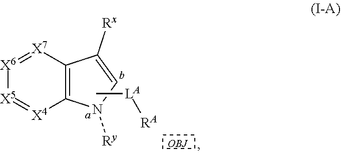
wherein
one of X⁴, X⁵, X⁶ or X⁷ is N and the remaining are CH;
R^{x} is selected from the group consisting of hydrogen; halo; and C₁₋₄alkyl optionally substituted with 1, 2 or 3 independently selected halo substituents;
R^{y} is absent when L^{A} is bound at position a of the 5-membered ring of the A-B 9-membered bicyclic heteroaryl system or is selected from hydrogen and C₁₋₄alkyl when L^{A} is bound at position b of the 5-membered ring of the A-B 9-membered bicyclic heteroaryl system;
and all other variables are as defined in any one of claims 1 to 3.

5. The compound of claim 4, having the Formula (I-A′) 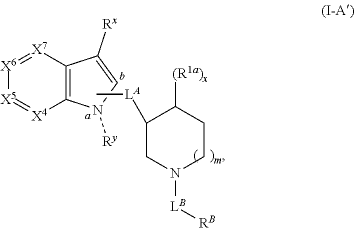
wherein
L^{A} is a bond or CH₂;
m is 0 or 1;
x is 0 or 1;
R^{1a} when present is C₁₋₄alkyl;
L^{B} is selected from the group consisting of >CH₂, >CH(CH₃), and >SO₂; in particular >CH₂ and >CH(CH₃); and
R^{B} is (b-1) or (b-4).

6. The compound according to claim 1, wherein 9-membered bicyclic heteroaryl system is selected from the group consisting of (ab-1), (ab-2), (ab-3), (ab-4) and (ab-5)

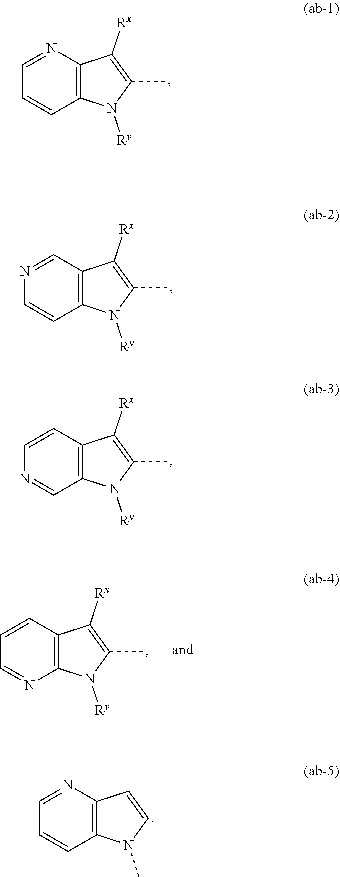

7. The compound of Formula (I) according to claim 1, having the Formula 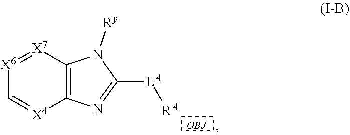
wherein
one of X⁴ or X⁷ is N and the other X⁷ or X⁴ is CH;
X⁶ is N or CR^{x} wherein R^{x} is selected from the group consisting of hydrogen; halo;
C₁₋₄alkyl optionally substituted with 1, 2 or 3 independently selected halo substituents; and C₁₋₄alkyloxy;
R^{y} is selected from hydrogen and C₁₋₄alkyl;
and all other variables are as defined in any one of claims 1 to 3.

8. The compound of claim 7, having the Formula (I-B′) 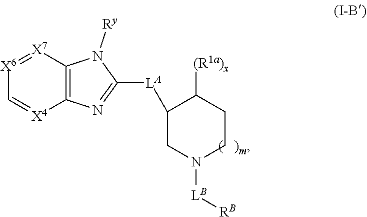
wherein
L^{A} is a bond or CH₂;
m is 0 or 1;
x is 0 or 1;
R^{1a} when present is C₁₋₄alkyl;
L^{B} is selected from the group consisting of >CH₂, >CH(CH₃), and >SO₂; in particular >CH₂ and >CH(CH₃); and
R^{B} is (b-1) or (b-4).

9. The compound according to claim 1, wherein the A-B 9-membered bicyclic heteroaryl system is selected from the group consisting of (ab-6), (ab-7), and (ab-8)

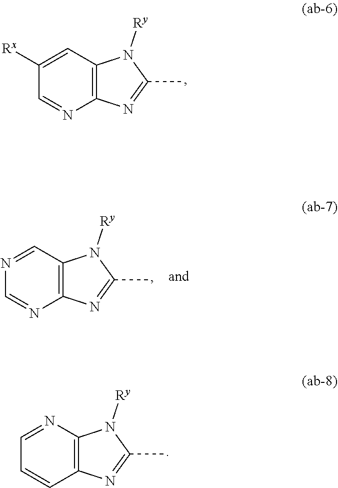

10. A pharmaceutical composition comprising a prophylactically or a therapeutically effective amount of a compound according to claim 1 and a pharmaceutically acceptable carrier.

11. A process for preparing a pharmaceutical composition comprising mixing a pharmaceutically acceptable carrier with a prophylactically or a therapeutically effective amount of a compound according to claim 1.

12. (canceled)

13. (canceled)

14. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according to claim 1.

15. A method for inhibiting O-GlcNAc hydrolase, comprising administering to a subject in need thereof, a prophylactically or a therapeutically effective amount of a compound according to claim 1.
